(19) United States
(12) Patent Application Publication
(10) Pub. No.: US 2026/0335111 A1
(43) Pub. Date: Oct. 29, 2026

(54) LOADING SYSTEM FOR IMPLANTABLE MEDICAL DEVICES

(71) Applicant: MEDTRONIC, INC., Minneapolis, MN (US)

(72) Inventors: Catherine Irvine, Brookeborough (GB); Daniel T. Costelloe, Galway (IE); Anna Rios Garriga, Wellpark (IE); David R. Nolan, Galway (IE); Patricia McAfee, Galway (IE); Kate Molan, Ennis (IE); Gavin Moore, London (GB); Patrick McFlynn, Galway (IE); Dmitry Sheremetiev, Oranmore (IE); Evelyn M. Collier, Galway (IE); Niall F. Duffy, Galway (IE); Niall Crosbie, Clare (IE); Ronan J. Benson, Sligo (IE); Jack F. Cullinan, Dublin (IE); Patrick Griffin, Galway (IE); Nicholas Fox, Oranmore (IE); Constantin Ciobanu, Galway (IE); David Francis, Claremorris (IE)

(73) Assignee: MEDTRONIC, INC., Minneapolis, MN (US)

(21) Appl. No.: 19/104,842

(22) PCT Filed: Aug. 22, 2023
(86) PCT No.: PCT/US2023/072667

§ 371 Date: Feb. 19, 2025

Related U.S. Application Data
(60) Provisional application No. 63/400,040, filed on Aug. 22, 2022.
(60) Provisional application No. 63/511,924, filed on Jul. 5, 2023.
(60) Provisional application No. 63/520,647, filed on Aug. 20, 2023.

(51) Int. Cl. A61F 2/95; A61F 2/24

(52) U.S. Cl. CPC A61F 2/9525; A61F 2/2427; A61F 2240/001

(57) ABSTRACT

A loading system for loading a heart valve prosthesis into a delivery system includes a loading cone, a loading ring, and a valve seat. The loading cone includes a passageway extending therethrough, with the passageway including a tapered portion. The loading ring is configured to be coupled to the loading cone. The valve seat is configured to be coupled to the loading ring. The valve seat is rotatable relative to the loading ring when the valve seat and the loading ring are coupled together. The valve seat is configured to receive the heart valve prosthesis.

### FIELD

The present technology is generally related to loading systems and methods for loading medical devices into a delivery device.

### BACKGROUND

Transcatheter implantable medical devices, such as heart valve prostheses, stents, scaffolds, and other intervention devices, are loaded into delivery devices to such as catheters to be delivered to a treatment site within a patient's vasculature. Some implantable medical devices that contain organic tissue, e.g., bovine and porcine, require onsite installation onto the delivery device. Thus, it is desirable to make loading of the implantable medical device onto the delivery device as easy as possible. Further, some current commercial systems in some cases may be subject to undesirable events during loading, such as valve inversion, valve infolding, and/or crown overlap. Thus, it is desirable to minimize the occurrence of such undesirable events.

### SUMMARY

In an example hereof, a loading system for loading a heart valve prosthesis into a delivery system includes a loading cone, a loading ring configured to be coupled to the loading cone, and a valve seat configured to be coupled to the loading ring. The loading cone includes a passageway extending therethrough, the passageway including a tapered portion. The valve seat is rotatable relative to the loading ring when the valve seat and the loading ring are coupled together. The valve seat is configured to receive the heart valve prosthesis.

In another example hereof, the loading system of any of the previous or subsequent examples further comprises a capsule guide tube, wherein a distal end of the capsule guide tube is configured to be disposed adjacent a proximal end of the loading cone.

In another example hereof, the loading system of any of the previous or subsequent examples further comprises a tip guide tube configured to extend through the loading ring, the valve seat, and the loading cone.

In another example hereof, in the loading system of any of the previous or subsequent examples, the tapered portion of the passageway is tapered in a proximal direction, such that a distal end of the tapered portion has a larger diameter than a proximal end of the tapered portion.

In another example hereof, in the loading system of any of the previous or subsequent examples, the tapered portion of the passageway is tapered at a taper angle of about 15 degrees to about 30 degrees, or about 15 degrees to about 250 degrees, about 18 degrees to about 22 degrees, or about 20 degrees.

In another example hereof, in the loading system of any of the previous or subsequent examples, the loading cone includes a plurality of ribs extending inwardly from an inner surface of the loading cone.

In another example hereof, in the loading system of any of the previous or subsequent examples, the loading ring includes a base and a central passageway extending through the base.

In another example hereof, in the loading system of any of the previous or subsequent examples, the loading ring further includes a slot configured to receive a corresponding locking arm of the loading cone to rotationally couple the loading ring and the loading cone.

In another example hereof, in the loading system of any of the previous or subsequent examples, the slot is defined by an inner wall, an outer wall, and radially extending side walls connecting the inner wall to the outer wall.

In another example hereof, in the loading system of any of the previous or subsequent examples, the slot comprises a plurality of slots, and wherein the loading cone includes a plurality of locking arms corresponding to the plurality of slots, wherein each slot extends circumferentially around a portion of a circumference of the base of the loading ring, and wherein the plurality of locking arms rotate relative to the plurality of slots to enable the loading cone to rotate relative to the loading ring.

In another example hereof, in the loading system of any of the previous or subsequent examples, the valve seat comprises a seat base having a central aperture extending therethrough, a wall extending from a periphery of the seat base in a first direction, and an aperture wall extending from a periphery of the central aperture in a second direction opposite the first direction, wherein the aperture wall is configured to be disposed in the central passageway of the loading ring such that the valve seat is rotatable relative to the loading ring.

In another example hereof, in the loading system of any of the previous or subsequent examples, the seat base and the wall of the valve seat are configured to receive the heart valve prosthesis.

In another example hereof, in the loading system of any of the previous or subsequent examples, the wall of the valve seat includes a plurality of ribs configured to aid in the valve seat engaging the heart valve prosthesis such that rotation of the valve seat results in corresponding rotation of the heart valve prosthesis.

In another example hereof, in the loading system of any of the previous or subsequent examples, the seat base includes a plurality of pockets configured to receive crowns at a first end of a frame of the heart valve prosthesis such that rotation of the valve seat results in corresponding rotation of the heart valve prosthesis.

In another example hereof, in the loading system of any of the previous or subsequent examples, the wall of the valve seat comprises two wall sections separated by two wall slots, wherein squeezing the aperture wall radially inwardly splays the two wall sections radially outwardly to receive the heart valve prosthesis therein.

In another example hereof, in the loading system of any of the previous or subsequent examples, the seat base of the valve seat further includes a living hinge configured to enable bending of the seat base to enable splaying of the two wall sections.

In another example hereof, in the loading system of any of the previous or subsequent examples, the loading cone, the loading ring, and the valve seat are configured to receive the heart valve prosthesis therein, and with the loading cone, the loading ring, and the valve seat coupled together and the heart valve prosthesis loaded therein, the loading cone, the valve seat, and the heart valve prosthesis are configured to rotate relative to the loading ring.

In another example hereof, in the loading system of any of the previous or subsequent examples, the aperture wall of the valve seat includes a circumferential lip such that with the valve seat coupled to the loading ring, the circumferential lip inhibits longitudinal movement between the valve seat and the loading ring.

In another example hereof, in the loading system of any of the previous or subsequent examples, the loading cone is non-rotatably coupled to the loading ring.

In another example hereof, in the loading system of any of the previous or subsequent examples, the loading cone, the loading ring, and the valve seat are configured to receive the heart valve prosthesis therein, and with the loading cone, the loading ring, and the valve seat coupled together and the heart valve prosthesis loaded therein, the valve seat and the heart valve prosthesis are configured to rotate relative to the loading ring and the loading cone.

In another example hereof, the loading system of any of the previous or subsequent examples further comprises a tip guide tube configured to engage an inner surface of the aperture wall of the valve seat, wherein rotation of the tip guide tube rotates the valve seat relative to the loading ring.

In another example hereof, in the loading system of any of the previous or subsequent examples, the tip guide tube includes a plurality of ridges configured to engage a corresponding plurality of slots in the aperture wall of the valve seat to rotatably engage the tip guide tube with the valve seat.

In another example hereof, a method of loading a heart valve prosthesis into a delivery system comprises: sliding a capsule guide tube over a delivery system such that a distal end of the capsule guide tube adjacent an open distal end of a capsule of the delivery system; coupling together a loading ring and a valve seat such that the valve seat is rotatable relative to the loading ring; loading the heart valve prosthesis into the valve seat; securing a loading cone to the loading ring with the valve seat secured to loading ring and the heart valve prosthesis disposed in the valve seat, thereby forming a loading system; loading the loading system onto the delivery system; rotating the valve seat and the heart valve prosthesis to align the heart valve prosthesis relative to the delivery system; coupling the heart valve prosthesis to the delivery system; translating the capsule distally to cover a portion of the heart valve prosthesis; and further translating the capsule distally to such that the capsule and capsule guide tube push the loading cone, loading ring, and valve seat distally as the heart valve prosthesis is prevented from translating distally by being coupled to the delivery system such that the loading cone radially compresses the heart valve prosthesis, the heart valve prosthesis exits a proximal end of the loading cone and enters an open distal end of the capsule.

In another example hereof, the method of any of the previous or subsequent examples further comprises securing the capsule guide tube to the capsule via friction.

In another example hereof, the method of any of the previous or subsequent examples further comprises inserting a tip guide tube through the loading ring, the valve seat, the transcatheter heart valve prosthesis, and the loading cone, such that a proximal end of the tip guide tube extends proximal of the proximal end of the loading cone.

In another example hereof, in the method of any of the previous or subsequent examples, the loading system further comprises the tip guide tube inserted through the loading ring, the valve seat, the heart valve prosthesis, and the loading cone, wherein loading the loading system onto the delivery system comprises inserting a distal tip of the delivery system into the proximal end of the tip guide tube and advancing the loading system proximally towards the capsule.

In another example hereof, in the method of any of the previous or subsequent examples, rotating the valve seat and the heart valve prosthesis comprises rotating the tip guide tube to rotate the valve seat, thereby rotating the heart valve prosthesis loaded into the valve seat.

In another example hereof, in the method of any of the previous or subsequent examples, the loading cone is non-rotatably coupled to the loading ring such that rotating the valve seat and the heart valve prosthesis does not rotate the loading cone.

In another example hereof, in the method of any of the previous or subsequent examples, the loading cone is rotatably coupled to the loading ring, wherein rotating the valve seat and the heart valve prosthesis comprises rotating the loading cone to rotate the heart valve prosthesis and the valve seat.

In another example hereof, the method of any of the previous or subsequent examples further comprises withdrawing the tip guide tube from the loading cone, the heart valve prosthesis, the valve seat, and the loading ring prior to translating the capsule distally to cover a portion of the heart valve prosthesis.

In another example hereof, in the method of any of the previous or subsequent examples, the valve seat comprises a seat base having a central aperture extending therethrough, a wall extending from a periphery of the seat base in a first direction, and an aperture wall extending from a periphery of the central aperture in a second direction opposite the first direction, wherein coupling the valve seat to the loading ring comprising inserting the aperture wall into a central passageway of the loading ring such that the valve seat is rotatable relative to the loading ring.

In another example hereof, in the method of any of the previous or subsequent examples, loading the heart valve prosthesis into the valve seat comprises inserting a portion of the heart valve prosthesis within the wall of the valve seat.

In another example hereof, in the method of any of the previous or subsequent examples, the seat base includes a plurality of pockets, wherein loading the heart valve prosthesis into the valve seat comprises inserting crowns at a first end of a frame of the heart valve prosthesis into the plurality of pockets.

In another example hereof, in the method of any of the previous or subsequent examples, the wall of the valve seat comprises two wall sections separated by two wall slots, wherein loading the heart valve prosthesis into the valve seat comprises squeezing the aperture wall radially inwardly to splay the two wall sections radially outwardly, inserting the heart valve prosthesis within the two wall sections in a splayed state, and releasing the aperture wall to return the two wall sections to an unsplayed state.

In another example hereof, in the method of any of the previous or subsequent examples, the seat base of the valve seat further includes a living hinge, wherein squeezing the aperture wall bends the seat base at the living hinge to, and wherein the living hinge returns the seat base to an unbent state when the aperture wall is released.

In another example hereof, in the method of any of the previous or subsequent examples, coupling the heart valve prosthesis to the delivery system comprises inserting tabs of a frame of the heart valve prosthesis into tab pockets of a spindle of the delivery system.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIG. 1A depicts a perspective, exploded illustration of a loading system for use with a medical device, according to embodiments hereof.

FIG. 1B depicts a cross-sectional illustration of the loading system of FIG. 1A, according to embodiments hereof.

FIG. 2 depicts an outflow cone and a loading ring of the loading system of FIGS. 1A and 1B coupled together.

FIGS. 3A and 3B depict illustrations of the loading ring of the loading system of FIGS. 1A and 1B, according to embodiments hereof.

FIGS. 4A through 4C depict illustrations of valve seats of the loading system of FIGS. 1A and 1B, according to embodiments hereof.

FIG. 5 depicts an illustration of an example transcatheter heart valve prosthesis which may be radially compressed and loaded into a delivery system using the loading systems described herein.

FIGS. 6A and 6B depict illustrations of an example delivery system into which a heart valve prosthesis may be loaded using the loading systems disclosed herein.

FIG. 7 depicts an illustration of an example loading tray with the example delivery system of FIGS. 6A and 6B for use in loading a transcatheter heart valve prosthesis using the loading systems described herein.

FIGS. 8A-8Y depict several illustrations of a method of loading a transcatheter heart valve prosthesis into a delivery system using the loading systems disclosed herein.

FIG. 9 depicts a longitudinal cross-sectional illustration of a loading system including an internal loading cone support.

FIG. 10 depicts a lateral cross-sectional illustration of the loading system including the inner loading cone support of FIG. 9.

FIGS. 11-13 depict a compliant exit of the loading cone of the loading system of FIG. 1 at various stages of the method of FIGS. 8A-8Y.

FIG. 14 depicts a perspective, exploded illustration of a loading system for use with a medical device, according to embodiments hereof.

FIG. 15 depicts a cross-sectional illustration of the loading system of FIG. 14, according to embodiments hereof.

FIGS. 16A-16D depict illustrations of a capsule guide tube of the loading system of FIG. 14, according to embodiments hereof.

FIGS. 17A-17B depict illustrations of loading cones of the loading system of FIG. 14, according to embodiments hereof.

FIGS. 18A-18B depict illustrations of a loading ring of the loading system of FIG. 14, according to embodiments hereof.

FIGS. 19A-19B depict illustrations of a valve seat of the loading system of FIG. 14, according to embodiments hereof.

FIG. 20 depicts an exploded view of a loading ring, valve seat, and loading cone of the loading system of FIG. 14, according to embodiments hereof.

FIG. 21 depicts a cross-sectional illustration of the loading cone, the loading ring, and the valve seat of the loading system of FIG. 14 assembled.

FIG. 22 depicts a perspective illustration of a tip guide tube of the loading system of FIG. 14, according to embodiments hereof.

FIG. 23 depicts a perspective illustration of the tip guide tube of FIG. 22 interacting with a valve seat of the loading system of FIG. 14, according to embodiments hereof.

FIG. 24 depicts a perspective view of a valve seat according to embodiments hereof.

FIG. 25 depicts a cross-sectional view of the valve seat of FIG. 24 according to embodiments hereof.

FIG. 26 depicts a cross-sectional view of the valve seat of FIG. 25 with wall sections thereof in a splayed state according to embodiments hereof.

FIG. 27 depicts a top perspective view of a valve seat according to embodiments hereof.

FIG. 28 depicts a bottom perspective view of the valve seat of FIG. 27.

FIG. 29 depicts a top view of the valve seat of FIG. 27

FIG. 30 depicts a side view of the valve seat of FIG. 27.

FIG. 31 depicts a top perspective view of a valve seat according to embodiments herein.

FIG. 32A depicts a perspective view of a valve seat according to embodiments herein.

FIG. 32B depicts a cut-away view of the valve seat of FIG. 32A.

FIG. 33A depicts a perspective view of a portion of a valve seat including protrusions extending from an inner surface of wall sections thereof.

FIG. 33A depicts a perspective view of a portion of a valve seat including protrusions extending from an inner surface of wall sections thereof.

FIG. 34A depicts a side view of a tip guide tube according to embodiments herein.

FIG. 34B depicts a perspective of the tip guide tube of FIG. 34A.

FIG. 35A depicts a perspective view of a tip guide tube according to embodiments herein.

FIG. 35B depicts a side view of the tip guide tube of FIG. 35A.

FIG. 36A depicts a perspective view of a tip guide tube according to embodiments herein.

FIG. 36B depicts a cross-sectional view of a portion of a valve seat for use with the tip guide tube of FIG. 36A.

FIG. 36C shows a cross-section view of a portion of the tip guide tube of FIG. 36A interacting with the valve seat of FIG. 36B.

FIG. 37 depicts a side view of a portion of a tip guide tube according to embodiments herein.

FIG. 38A depicts a perspective view of a tip guide tube according to embodiments herein.

FIG. 38B depicts a perspective view of the tip guide tube of FIG. 38A interacting with a valve seat.

FIG. 38C depicts a close-up side view of the tip guide tube of FIG. 38A interacting with a valve seat.

FIG. 39A depicts a side view of a tip guide tube according to embodiments herein.

FIG. 39B depicts a perspective of the tip guide tube of FIG. 39A.

FIG. 40A depicts a perspective view of a tip guide tube according to embodiments herein.

FIG. 40B depicts a perspective view of a loading system with the tip guide tube of FIG. 40A.

FIG. 41A depicts a perspective view of a pusher for a loading system according to embodiments herein.

FIG. 41B depicts an exploded view of a loading system with the pusher of FIG. 41A.

FIGS. 42A and 42B show side views of a tip guide tube and a loading ring according to embodiments herein.

FIGS. 43A and 43B show longitudinal cross-sectional views of a tip guide tube including an internal spring according to embodiments herein.

FIG. 44A shows perspective view of a tip guide tube including an external spring according to embodiments herein.

FIG. 44B shows a cross-sectional view of the tip guide tube of FIG. 44A disposed through a loading system according to embodiments herein.

FIGS. 45A and 45B show a perspective view and a longitudinal cross-sectional view of a tip guide tube with a flared end according to embodiments herein.

FIG. 46 shows a perspective view of a tip guide tube according to embodiments herein.

FIG. 47 shows a side view of a tip guide tube including cut-outs according to embodiments herein.

FIG. 48A shows a schematic view of a tip guide tube according to embodiment herein.

FIG. 48B shows a schematic view of the tip guide tube of FIG. 48A interacting with a loading ring of a loading system according to embodiments herein.

FIGS. 49A-49E show a capsule guide tube according to embodiments herein.

FIG. 50A shows a perspective view of a capsule guide tube according to embodiments herein.

FIG. 50B shows a portion of a tube of the capsule guide tub of FIG. 50A according to embodiments herein.

FIGS. 51A-51C show cross-sectional views of a capsule guide tube according to embodiments herein.

FIGS. 52A-52C show a capsule guide tube according to embodiments herein.

FIGS. 53A-53C show perspective views of a capsule guide tube according to embodiments herein.

FIGS. 54A-54B show views of a locking member of a capsule guide tube according to embodiments herein.

FIG. 55 shows a perspective view of a locking member of a capsule guide tube according to embodiments herein.

FIG. 56 shows a perspective view of a locking member of a capsule guide tube according to embodiments herein.

FIG. 57 shows a perspective view of a loading cone of a loading system according to embodiments herein.

FIG. 58 shows a perspective view of a loading cone of a loading system according to embodiments herein.

### DETAILED DESCRIPTION

Specific embodiments of the present disclosure are now described with reference to the figures, wherein like reference numbers indicate identical or functionally similar elements.

The following detailed description describes examples of embodiments and is not intended to limit the present technology or the application and uses of the present technology. Although the description of embodiments hereof is in the context of a loading device for a transcatheter heart valve prosthesis, the present technology may also be used for other devices. Furthermore, there is no intention to be bound by any expressed or implied theory presented in the preceding technical field, background, brief summary or the following detailed description.

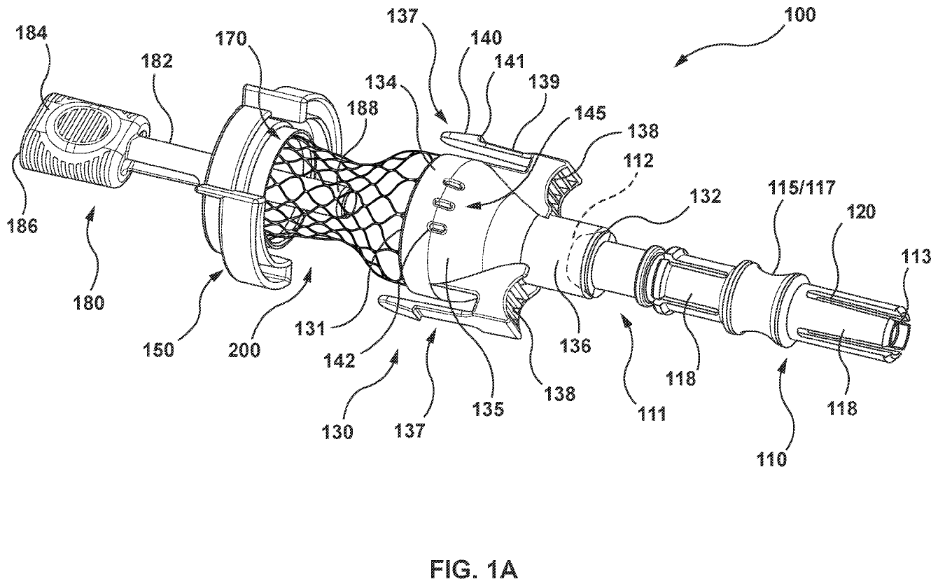

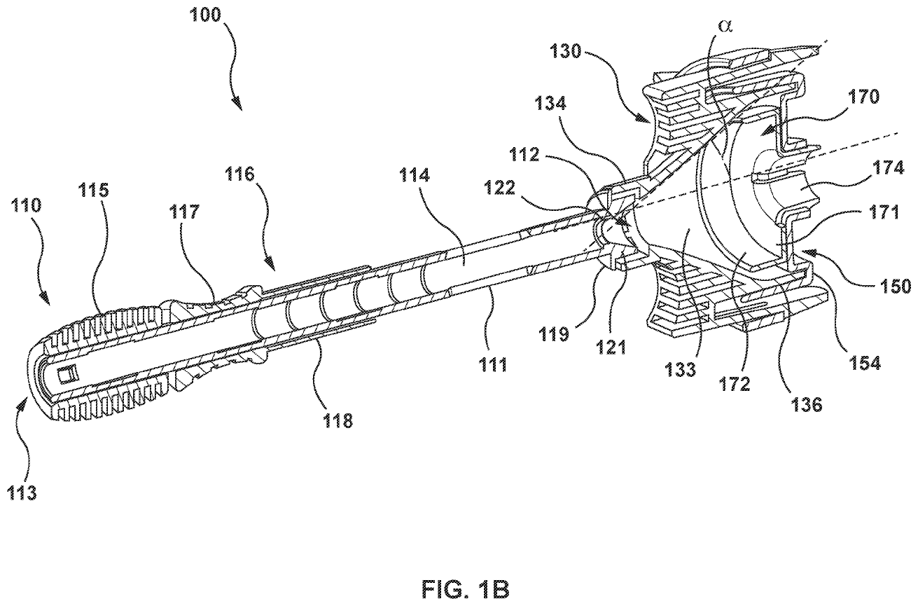

FIGS. 1A and 1B illustrate an example of a loading system 100 in accordance with an embodiment hereof. One skilled in the art will realize that FIGS. 1A and 1B illustrate one example of a loading system and that existing components illustrated in FIGS. 1A and 1B may be removed and/or additional components may be added to the loading system 100. As illustrated in FIG. 1A, the loading system 100 includes a capsule guide tube 110, a loading cone 130, a loading ring 150, a valve seat 170, and a tip guide tube 180. Further, it is noted that FIGS. 1A and 1B are depicted in opposite directions. In other words, the capsule guide tube 110 is shown to the right of the loading cone 130 in FIG. 1A, but to the left of the loading cone 130 in FIG. 1B. This is merely a perspective change, and the relative direction of parts with respect to each other are unchanged.

The capsule guide tube 110 is configured to be disposed on a capsule of a delivery system, as explained in more detail below. The capsule guide tube 110 includes a tube 111 having a first end 112, a second end 113, and a passageway or lumen 114 extending from the first end 112 to the second end 113, as shown in FIG. 1B. The capsule guide tube 110 further includes a grip 115 coupled to an exterior of the tube 111, and a locking member 116 configured to lock the capsule guide tube 110 to the capsule of the delivery system, as explained in more detail below. The locking member 116 is disposed around the tube 111 and includes a lock grip 117 and a lock tube 118 extending from the lock grip 117 towards the first end 112 of the capsule guide tube 110. The locking member 116 is slidable over the tube 111 and exerts a radially inward force on the tube 111 to lock the capsule guide tube 110 onto the capsule of the delivery device, as explained in more detail below. FIG. 1A shows a variation of the capsule guide tube 110 in which the grip 115 is combined with the lock grip 117. Further, the lock tube 118 of the embodiment of FIG. 1A extends from the grip/lock grip 115/117 in both towards the first end 112 and the second end 113 of the tube 111. The lock tube 118 of the embodiment of FIG. 1A further includes slits 120 between portions thereof. As explained above, and in more detail below, the capsule guide tube 110 of both FIG. 1A and FIG. 1B locks onto the capsule of the delivery device.

As shown in FIG. 1B, the first end 112 of the capsule guide tube 110 may include a shoulder 119 that is configured to interact with a second end of the loading cone 130 to prevent the tube 111 from extending into the loading cone 130 beyond a certain distance. A portion 121 of the lumen 114 of the tube 111 of the capsule guide tube 110 between the shoulder 119 and the first end 112 thereof is tapered in a direction away from the first end 112 such that the opening at the first end 112 is larger than the lumen 114 at the shoulder 119. This taper is such that the first (distal) end 112 of the lumen of the capsule guide tube 110 is larger, allowing the distal end of the capsule of the delivery system to expand during loading of the transcatheter heart valve prosthesis into the capsule. This reduces tissue compression during loading. Further, expansion (flaring) of the distal end of the capsule may reduce the likelihood of the crowns of the transcatheter heart valve prosthesis from catching on the capsule distal end during loading. Further, the lumen 114 at the shoulder 119 is smaller than the lumen 114 towards the second end 113 of the capsule guide tube 110 due to a lip 122 extending radially inward into the lumen 114, as shown in FIG. 1B. The lip 122 acts as a ring gauge to prevent incorrect loading of the transcatheter heart valve prosthesis. In other words, the inner diameter of the tube 111 of the capsule guide tube 110 is nominally the same as the inner diameter of the capsule of the delivery system, described below. Thus, there is insufficient space for the tabs of the heart valve prosthesis to sit above the tab pockets of the spindle of the delivery system. The lip 122 thus minimizes the likelihood of misloads into the delivery system.

The loading cone 130 includes a main body 145 including a first end 131, a second end 132, and a central passageway 133 extending from the first end 131 to the second end 132, as shown in FIGS. 1A and 1B. The loading cone 130 described herein is sometimes referred to as an outflow cone because in use with the transcatheter heart valve prosthesis 200 described herein for replacing a native aortic heart valve, and via transcatheter delivery via the aorta, the outflow portion of the transcatheter heart valve prosthesis 200 is initially loaded into the loading cone 130, as described in more detail below. However, this is not meant to be limiting, and with other devices, other native valves, and/or other delivery routes, the inflow portion of a medical device may be initially loaded into the loading cone 130. The loading cone 130 may also be referred to as a funnel and/or a loading funnel.

The main body 145 of the loading cone 130 includes a first cylindrical portion 134 adjacent the first end 131, a second cylindrical portion 136 adjacent the second end 132, and a tapered portion 135 disposed between the first cylindrical portion 134 and the second cylindrical portion 136. Although described as cylindrical portions, the first and second cylindrical portions 134, 136 need not be cylindrical and can be other similar shapes. In the embodiments shown, both the outer surface and the central passageway 133 in the tapered portion 135 are tapered. However, this is not meant to be limiting, and only the central passageway 133 of the tapered portion 135 needs to be tapered. The tapered portion 135 is tapered in a direction towards the second end 132 of the loading cone 130 such that the central passageway 133 of tapered portion 135 adjacent the first cylindrical portion 134 is larger than the central passageway 133 of the tapered portion 135 adjacent the second cylindrical portion 136. The tapered portion 135 of the central passageway is tapered at a taper angle α of approximately 20 degrees. However, this is not meant to be limiting, and the taper angle α may be in the range of 15-30 degrees, or 15-25 degrees, or 18-22 degrees. This taper angle α is smaller than other loading cones in the market, thereby limiting potential damage to valve tissue during loading of the transcatheter heart valve prosthesis into the delivery system.

The loading cone 130 may also include air vents 142, which are openings extending from an outer surface of the main body 145 to the central passageway 133 of the main body 145 of the loading cone 130. In the embodiment shown, the main body 145 includes a total of six air vents 142 in the first cylindrical portion 134 thereof. However, this is not meant to be limiting, and more or fewer air vents 142 may be utilized in suitable locations of the main body 145. In other embodiments, the main body 145 does not include the air vents.

In the embodiment shown, the loading cone 130 further includes two locking arms 137 extending radially outward from and longitudinally towards the first end 131 of the main body 145. In the embodiment shown, the two locking arms 137 are disposed 180 degrees apart from each other around the circumference of main body 145. Each of the locking arms 137 includes a support 138 extending radially outward from the outer surface of the main body 145 and an arm 139 extending longitudinally from the support 138 towards the first end 131 of the main body 145. Each arm 139 includes a distal portion (distal relative to the support 138) that is tapered from a first thickness adjacent a lip 141 to a second thickness, smaller than the first thickness, at a distal end of the arm 139. In other words, the distal portion of each arm 139 includes a ramp between the lip 141 and the distal end of the arm 139. The locking arms 137 are configured to couple the loading cone 130 with the loading ring 150, as described in more detail below.

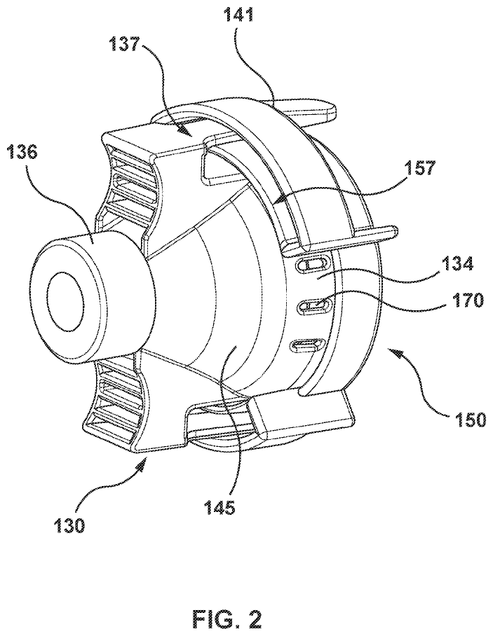

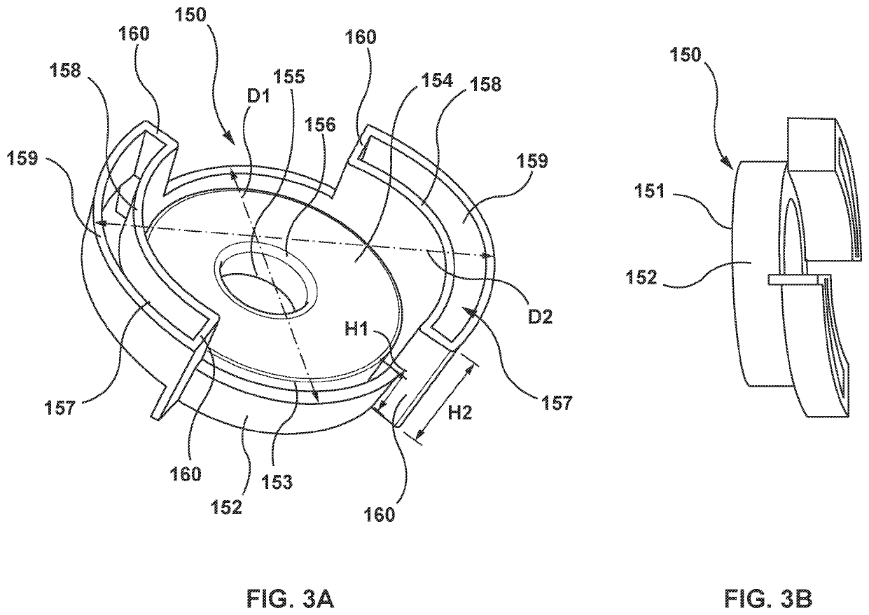

As noted above, the loading system 100 further includes the loading ring 150. The loading ring 150 is configured to be coupled to the loading cone 130 and to seat the transcatheter heart valve prosthesis 200 therein, as shown in FIGS. 1A, 1B, and 2. As shown in FIGS. 3A-3B, the loading ring 150 includes a base 151, an outer wall 152 extending generally perpendicularly from the base 151, and an inner wall 153 extending generally perpendicularly from the base 151 in the same direction as the outer wall 152, but spaced radially inward therefrom. In the embodiment shown, the base 151 is substantially circular, and the outer wall 152 and the inner wall 153 are substantially cylindrical. The loading ring 150 further includes a platform 154 extending radially inward from the inner wall 153 opposite the base 151. The loading ring 150 further includes a central lumen or passageway 155 extending from the platform 154 to the base 151. A cylindrical wall 156 defines the central passageway 155.

Still referring to FIGS. 3A and 3B, the loading ring 150 further includes slots 157, with each slot 157 configured to receive a corresponding locking arm 137 of the loading cone 130 to rotationally couple the loading ring 150 and the loading cone 130. In the embodiment shown, there are two slots 157 in the loading ring 150, corresponding to the two locking arms of the loading cone 130. Each slot 157 is defined by an inner wall 158, an outer wall 159, and radially extending side walls 160 connecting the inner wall 158 to the outer wall 159. The inner wall 158 of each slot 157 includes a portion of the outer wall 152 of the loading ring 150 and continues to extend from the outer wall 152. In particular, the inner wall 158 of each slot 157 includes a portion of the outer wall 152 and extends upwardly (i.e., away from the base 151) therefrom. The outer wall 159 of each slot 157 is spaced radially outward from the inner wall 158 of each slot 157 relative to the central passageway 155. Further, the inner wall 158 and the outer wall 159 of each slot 157 extend only partially around a circumference defined by the respective wall if the wall were extended circumferentially to define a full circle. In particular, each inner wall 158 and each outer wall 159 may extend 40-60 degrees around the circumference of such a circle. In other embodiments (not shown), rather than two slots 157, the loading ring 150 may include a single continuous slot that extends around the entire loading ring 150. In other embodiments, the loading ring 150 may include more than two slots 157, such as three slots, four slots, or more than four slots. In some embodiments the number of locking arms 137 of the loading cone 130 may match the number of slots 157 in the loading ring 150. In other embodiments, the number of locking arms need not match the number of slots 157.

In embodiments, the loading ring 150 has a first diameter D1 defined by the outer wall 152 of about 40-45 mm. The loading ring 150 has a second diameter D2 defined by the outer walls 159 of the slots 157 of about 42-46 mm. Further, the outer wall 152 of the loading ring 150 may have a height H1 of about 20 mm, the inner and outer walls 158, 159 of the slots 157 may have a height H2 of about 50 mm, and the inner wall 153 of the loading ring 150 may have a height of about 3.5 mm.

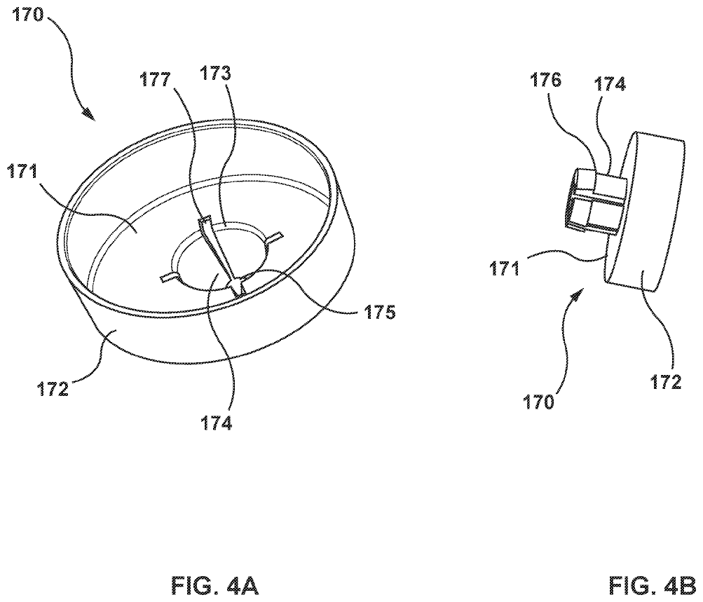

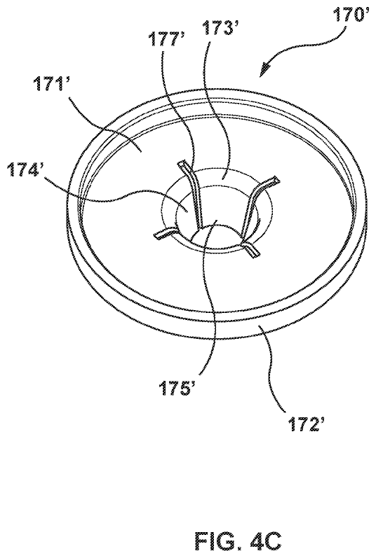

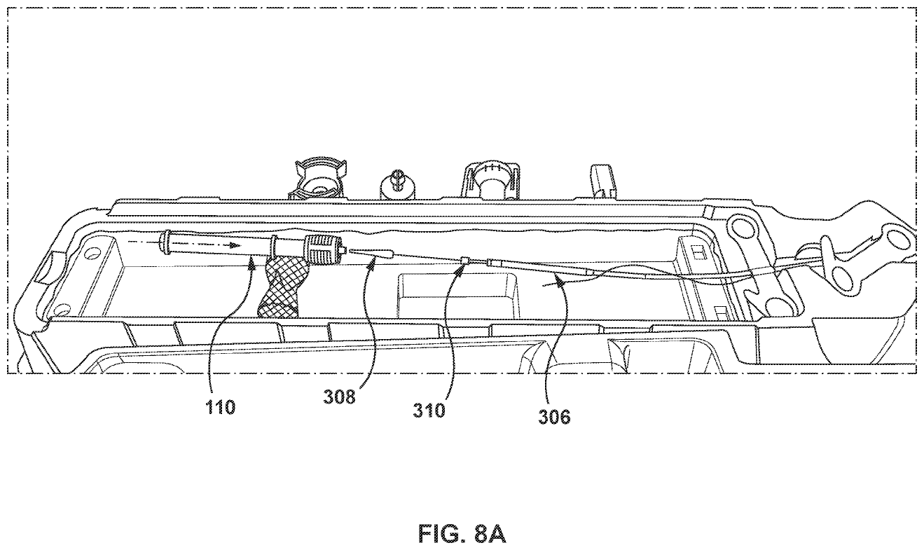

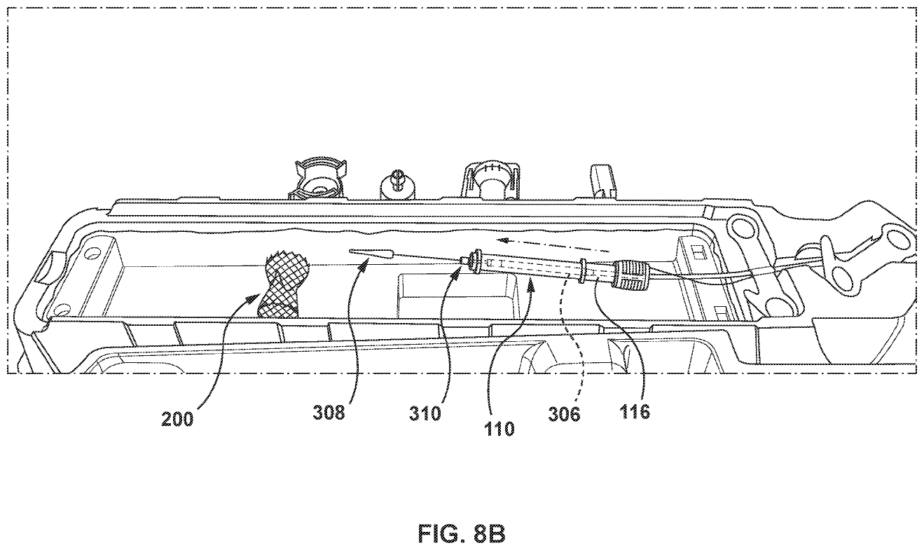

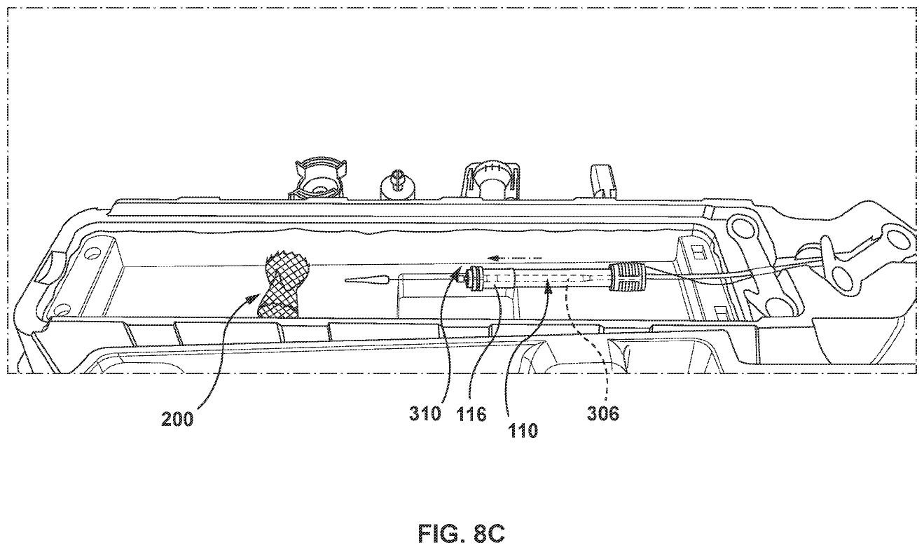

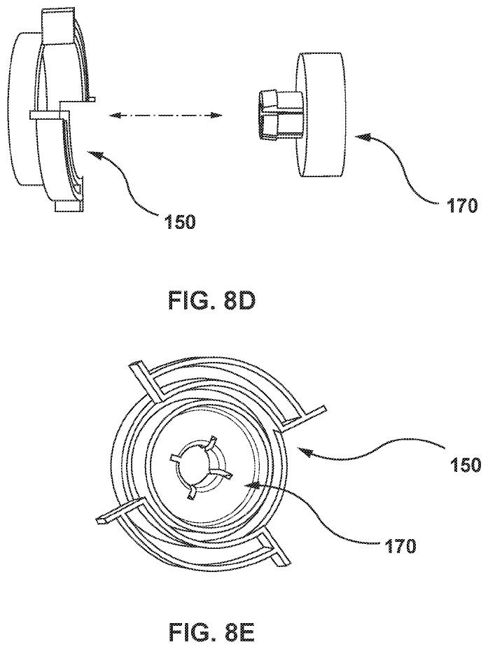

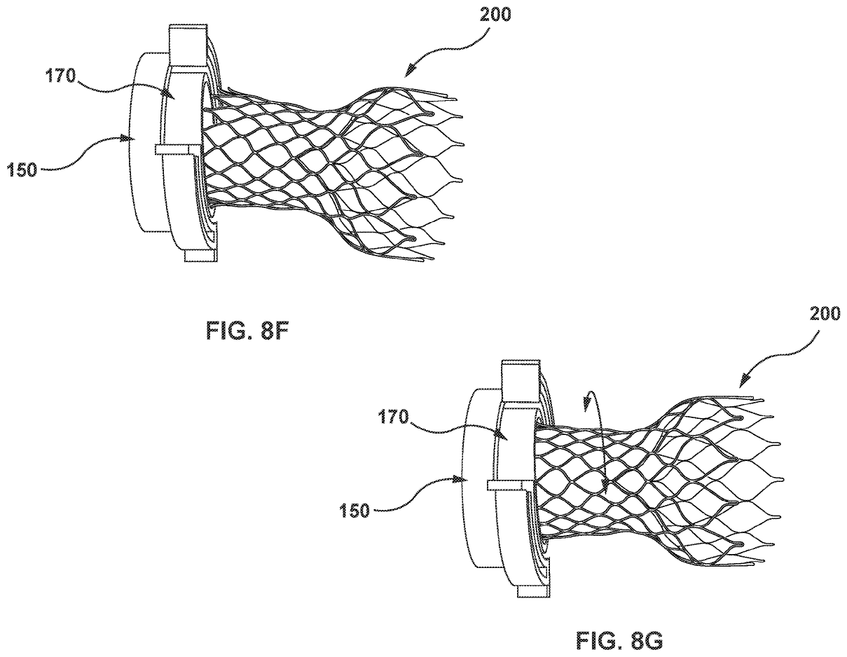

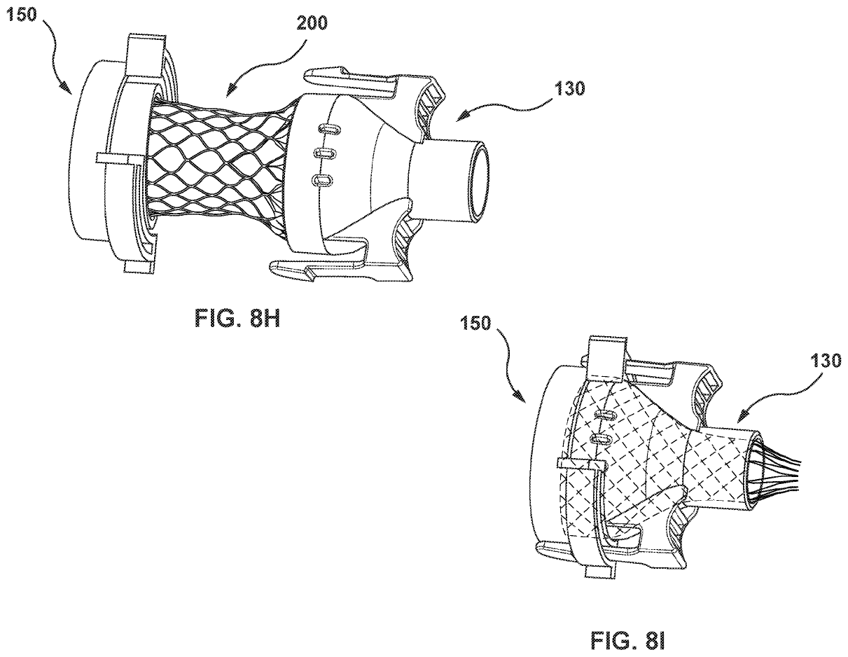

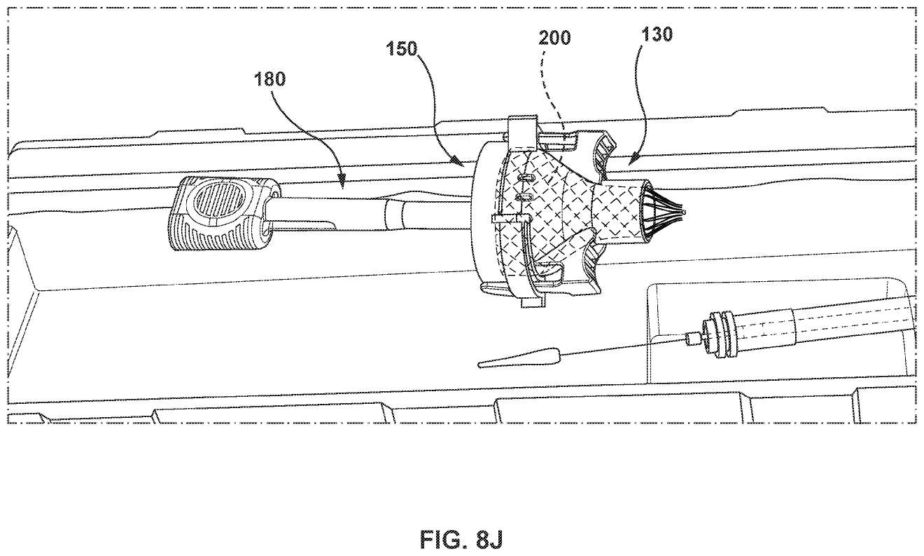

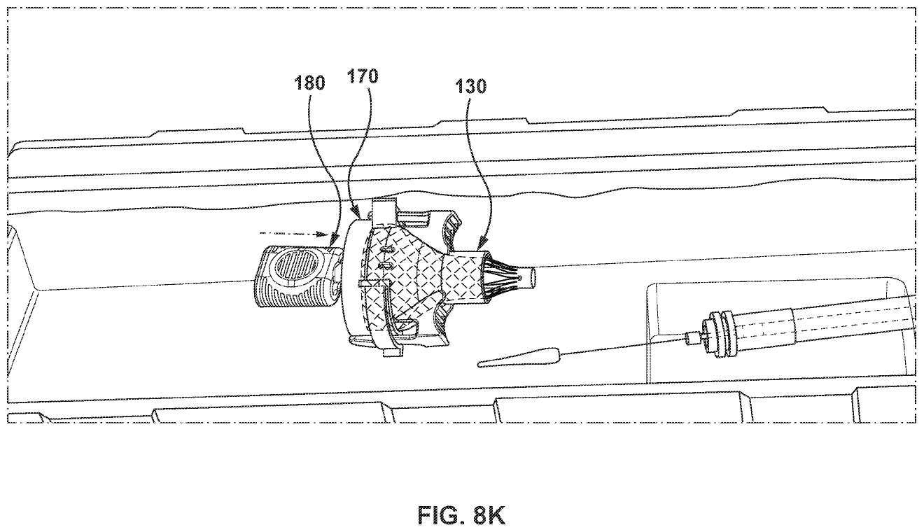

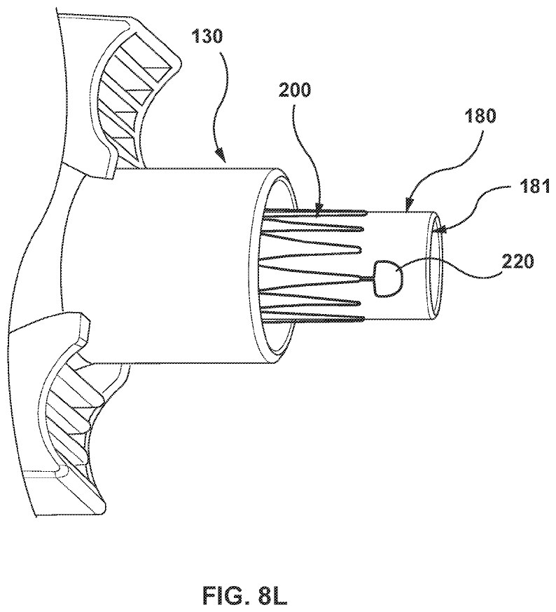

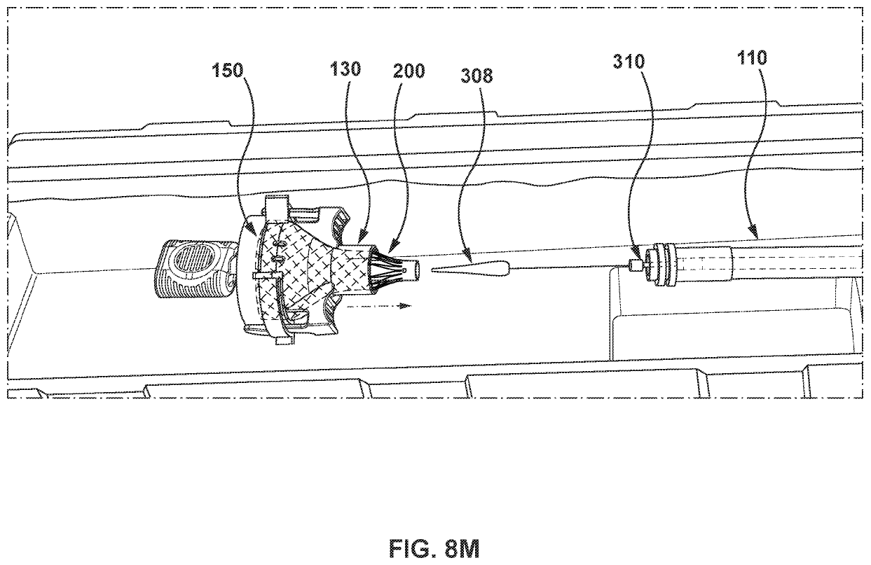

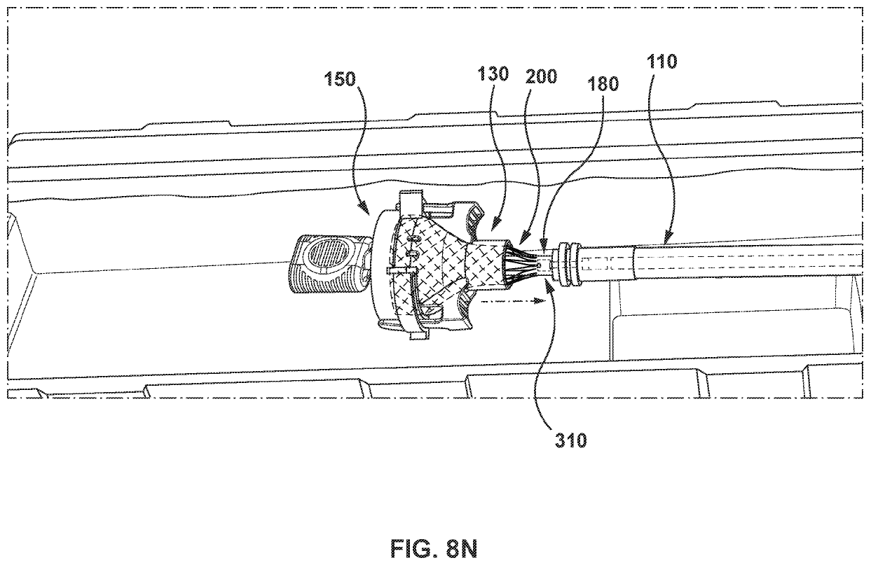

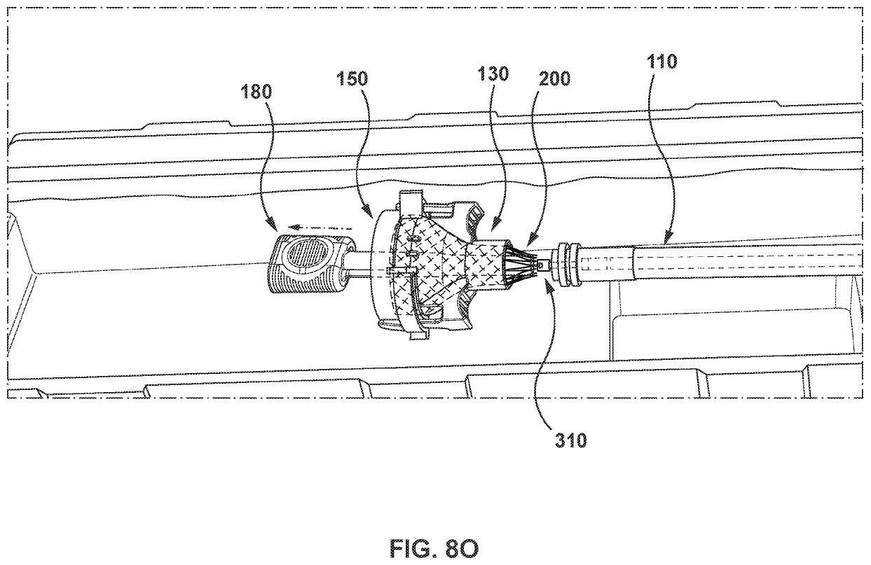

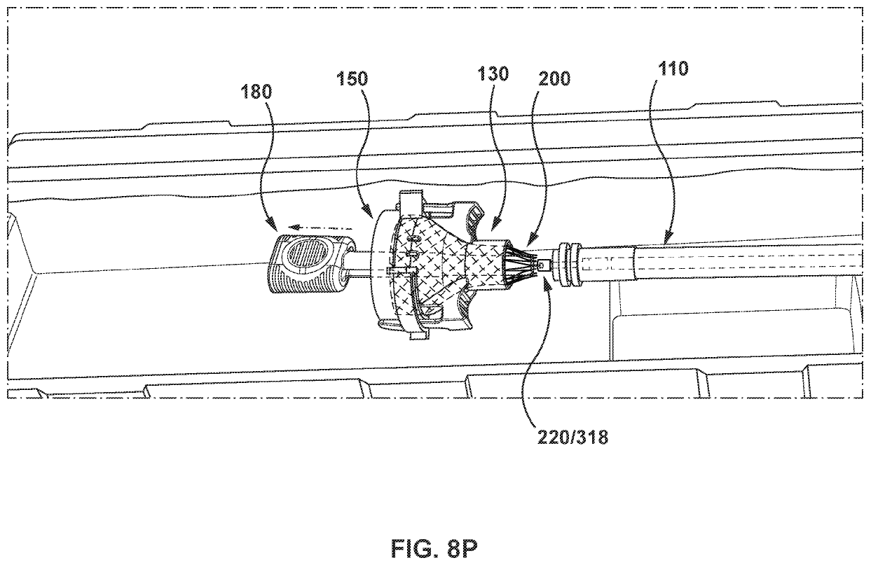

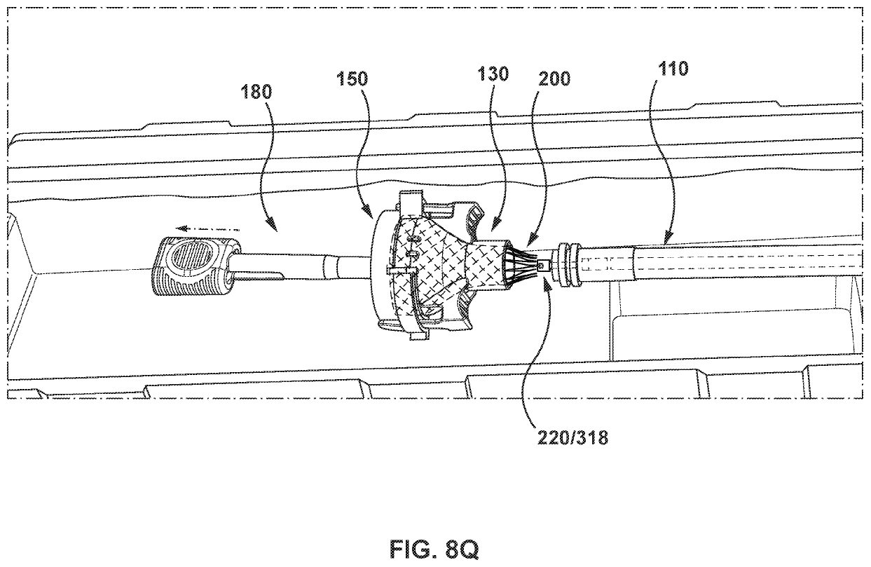

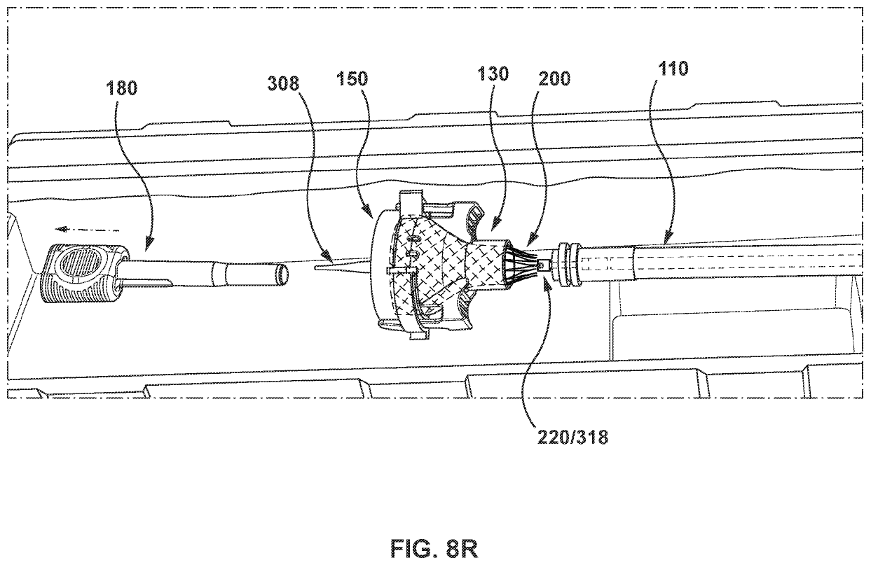

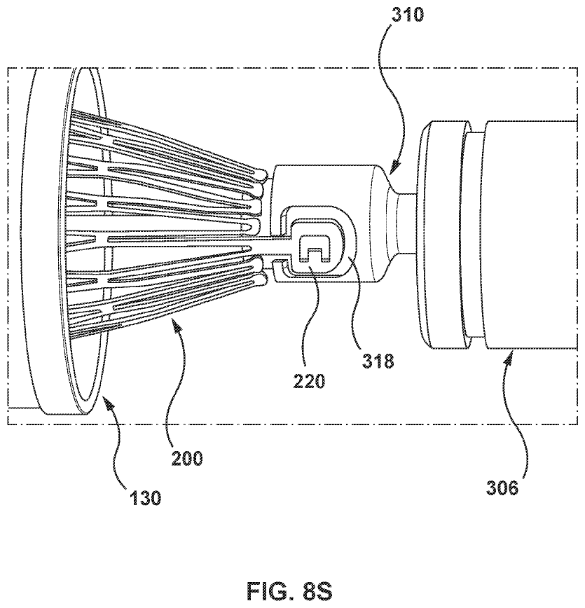

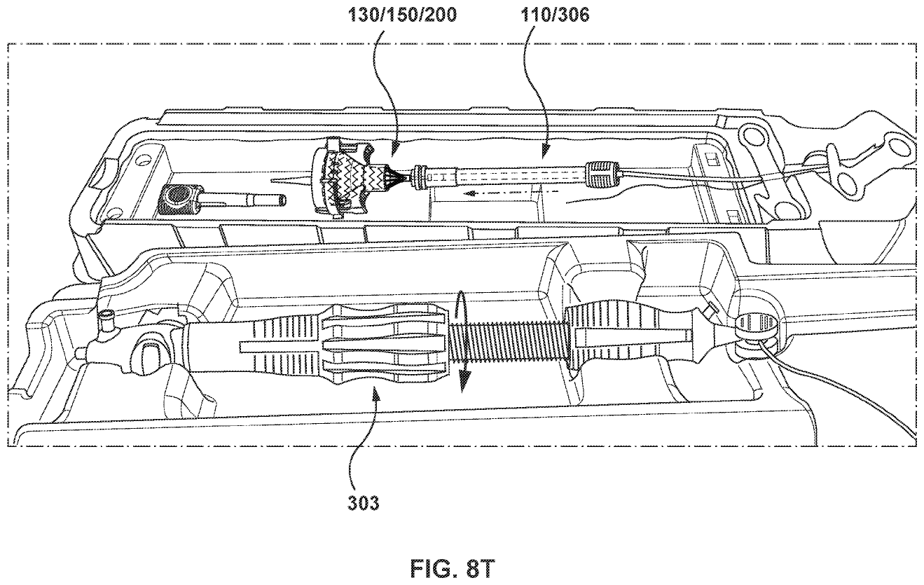

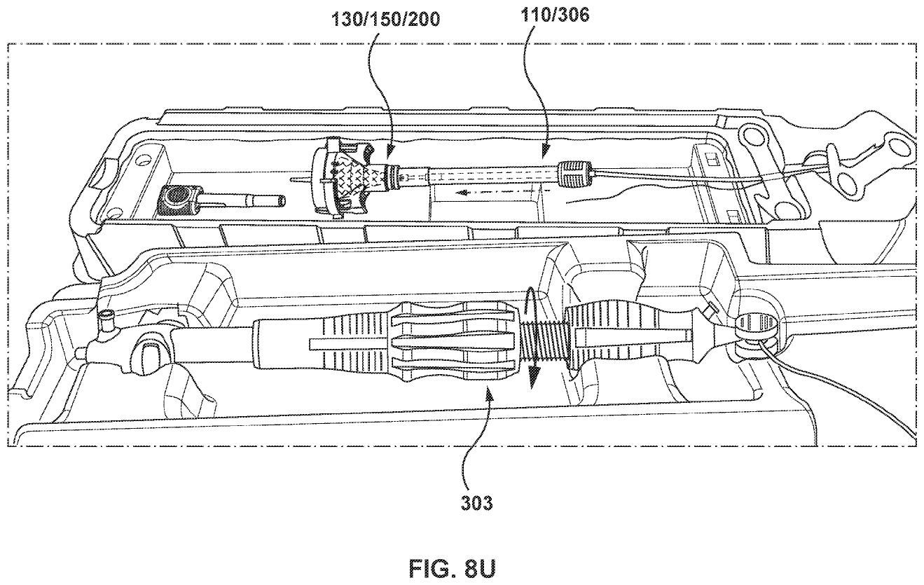

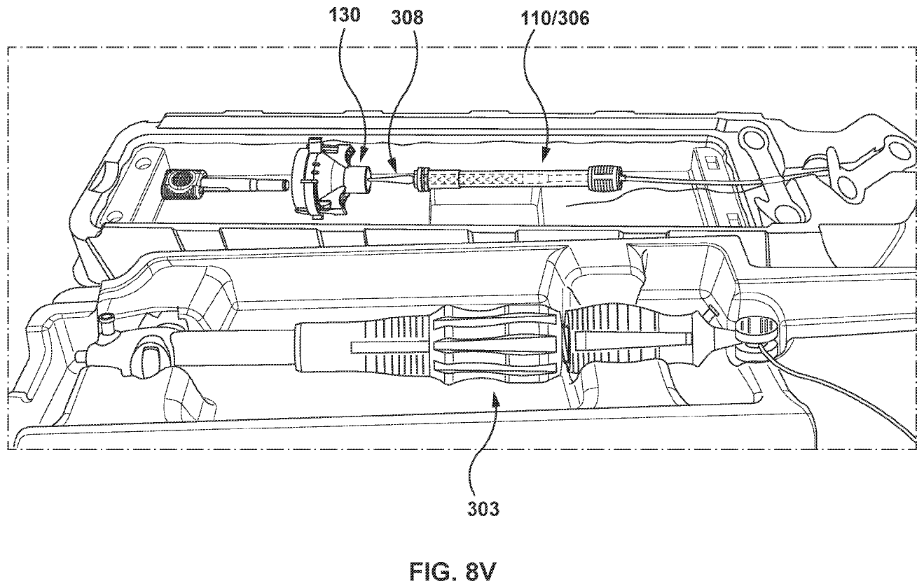

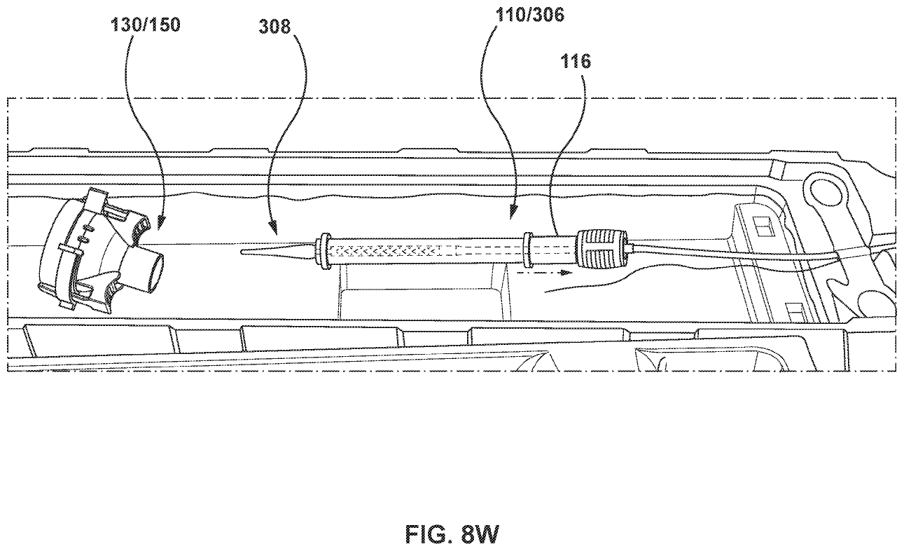

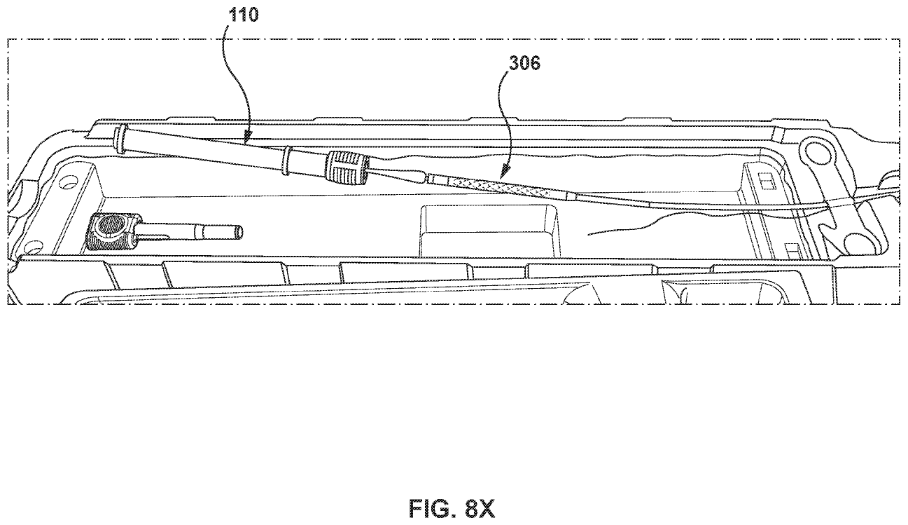

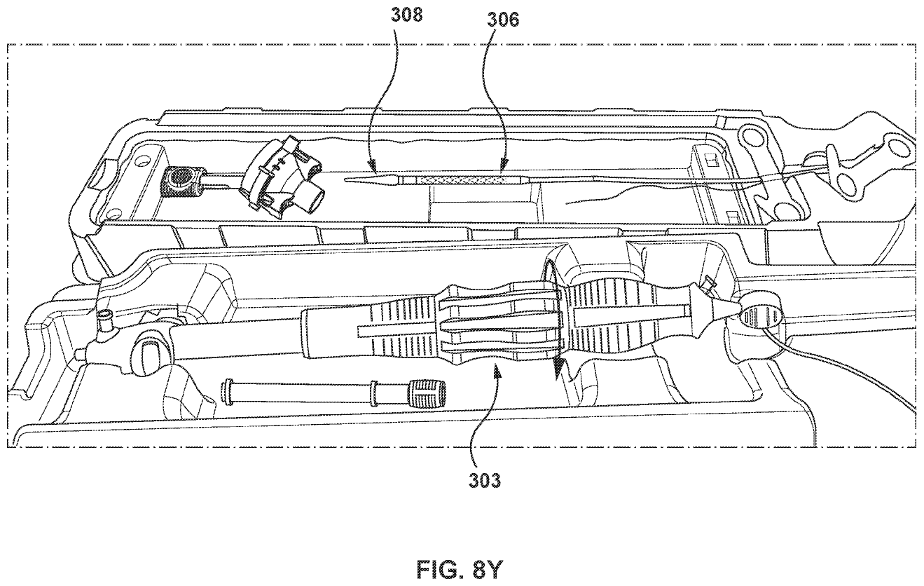

FIGS. 4A and 4B show an embodiment of a valve seat 170 of the loading system 100. The valve seat 170 includes a base 171 having a central aperture 173 and a wall 172 extending from a periphery of the base 171 in a first direction generally perpendicular to the base 171. The valve seat 170 further includes an aperture wall 174 extending from a periphery of the aperture 173 in a second direction, opposite the first direction, and generally perpendicular to the base 171. A central passageway 175 is defined by the aperture wall 174 and extends in the second direction from the central aperture 173 of the base 171. The aperture wall 174 may include a lip 176 or similar feature to couple the valve seat 170 to the loading ring 150, as described in more detail below. In particular, the aperture wall 174 of the valve seat 170 extends through the central passageway 155 the loading ring 150 until the lip 176 of the aperture wall 174 extends past the wall 156 defining the central passageway 155 such that the lip 176 can prevent longitudinal movement of the valve seat 170 with respect to the loading ring 150. However, the valve seat 170 is rotatable relative to the loading ring 150 because the aperture wall 174 is rotatable within the wall 156 of the loading ring 150. If the valve seat 170 and the loading ring 150 need to be separated from each other, the aperture wall 174 of the valve seat 170 may be squeezed radially inward, which may be made easier by slots 177 in the aperture wall 174, to enable the lip 176 to clear the end of the wall 156. FIG. 8E, described below, shows the loading ring 150 and the valve seat 170 coupled to each other.

FIG. 4C shows another embodiment of a valve seat 170′. The valve seat 170′ includes the same basic features of the valve seat 170, such as but not limited to, a base 171′ having a central aperture 173′, a wall 172′, an aperture wall 174′ defining a central passageway 175′. As in the embodiment of FIGS. 4A and 4B, the aperture wall 174′ may include a lip (not shown) or similar feature to couple the valve seat 170′ to the loading ring 150. The valve seat 170′ is shown herein to show that the same loading cone 130, loading ring 150, tip guide tube 180, and capsule guide tube 110 can be used with various sizes of transcatheter heart valve prostheses by changing the valve seat. However, only two examples of valve seats have been provided, and other embodiments may also be used. By varying features of the valve seat 170, such as but not limited to the height of the wall 172 and the diameter of the base 171, various transcatheter heart valve prostheses may be loaded into a delivery system using the same capsule guide tube 110, loading cone 130, loading ring 150, and tip guide tube 180.

FIG. 2 shows the loading ring 150, with the valve seat 170 coupled thereto (partially seen through the air vents 142), coupled to the loading cone 130. In order to couple the loading cone 130 and the loading ring 150 together, the arms 137 of the loading cone 130 are disposed through the slots 157 of the loading ring 150. The loading cone 130 and the loading ring 150 are brought together such that the first end 131 of the main body 145 of the loading cone 130 is disposed between the inner wall 153 and the outer wall 152 of the loading ring 150. The loading cone 130 and the loading ring 150 are brought together until the lip 141 of each locking arm 137 extends past the outer wall 159 of the respective slot 157, as shown in FIG. 2. This prevents longitudinal movement of the loading cone 130 with respect to the loading ring 150, but allows for rotational movement between them. Further, in order to disengage the loading cone 130 from the loading ring 150, the arms 139 may be pushed radially inward such that the lips 141 are radially inward of the outer walls 159 of the slots 157, thereby no longer preventing longitudinal movement of the loading cone 130 and the loading ring 150 away from each other.

As noted above, the loading system 100 further includes the tip guide tube 180, as shown in FIGS. 1A and 1B. The tip guide tube 180 includes a tube 182 and a grip 184. In the embodiment of FIGS. 1A and 1B, the tube 182 and the grip 184 are a single piece, however, this is not meant to be limiting. The tip guide tube 180 includes a first end 186 at the grip end of the tip guide tube 180 and a second end 188 opposite the first end 186. The tube 182 further includes a lumen 181 (see FIG. 8L) disposed as least partially therethrough. The lumen is configured to receive a distal tip of a delivery system during the loading process, as described below. The tube 182 is sized laterally (diameter) such that it fits through the central passageway 175 of the valve seat 170. The tip guide tube 180 has a longitudinal length such that with the loading cone 130 and the loading ring 150 coupled to each other, and the grip 184 abutting the base 151 of the loading ring 150, the tube 182 extends through the loading ring 150 and the loading cone 130 such that the second end 188 thereof protrudes from the second end 132 of the main body 145 of the loading cone 130, as can be seen in FIG. 8K.

With the loading system 100 having been described, a brief description of an example transcatheter heart valve prosthesis 200 and a delivery system 300 will be provided. The heart valve prosthesis 200 is merely an example heart valve prosthesis that the loading system 100 can be used on to load the heart valve prosthesis 200 into the delivery system 300, which is also merely an example. Other heart valve prostheses and delivery systems may be used in conjunction with the loading system to radially compress and load such heart valve prostheses into such delivery systems.

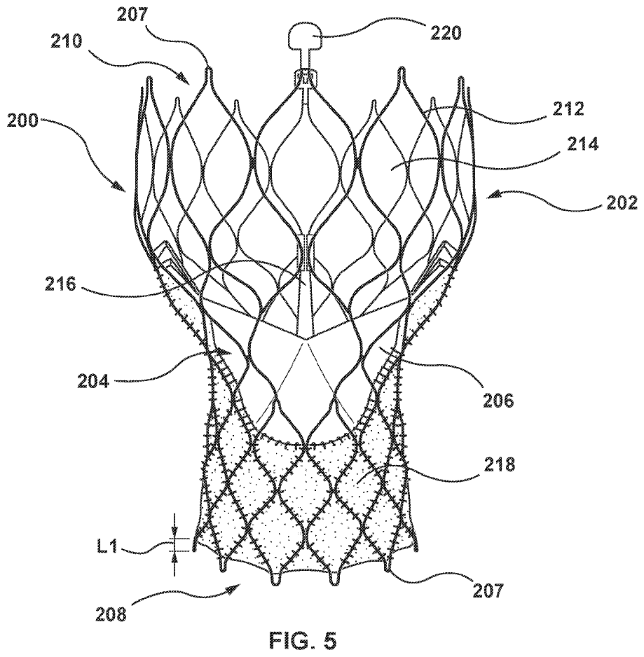

The transcatheter heart valve prosthesis 200, as shown in FIG. 5, generally includes a radially-expandable stent or frame 202 and a prosthetic valve 204. The frame 202 of the transcatheter heart valve prosthesis 200 supports the prosthetic valve 204 within the interior of the frame 202. In the example transcatheter heart valve prosthesis 200 shown in FIG. 5, the frame 202 is self-expandable. However, this is not meant to be limiting, and the frame 202 can be balloon-expandable or mechanically expandable. The transcatheter heart valve prosthesis 200 includes an inflow end 208 and an outflow end 210. One skilled in the art will realize that FIG. 5 illustrates one example of a transcatheter heart valve prosthesis and that existing components illustrated in FIG. 5 may be removed and/or additional components may be added to the transcatheter heart valve prosthesis 200.

The prosthetic valve 204 includes at least one leaflet 206 disposed within and secured to the frame 202. In the exemplary embodiment shown in FIG. 5, the prosthetic valve 204 includes exactly three leaflets 206. However, this is not meant to be limiting, as the prosthetic valve 204 may include more or fewer leaflets 206. The valve leaflets 206 open and close to regulate flow through the transcatheter heart valve prosthesis 200. Adjacent prosthetic leaflets 206 at joined together at commissures 216, which are attached to the frame 202 to couple the prosthetic valve 204 to the frame 202.

As shown in FIG. 5, the transcatheter heart valve prosthesis 200 includes an inflow end 208 and an outflow end 210. The prosthetic leaflets 206 are attached to the frame 202 such that when pressure at the inflow end 208 exceeds pressure at the outflow end 210, the prosthetic leaflets 206 open to allow blood flow through the heart valve prosthesis 200 from the inflow end 208 to the outflow end 210. When the pressure at the outflow end 210 exceeds pressure at the inflow end 208, the prosthetic leaflets 206 close to prevent blood flow from the outflow end 210 to the inflow end 208. The transcatheter heart valve prosthesis 200 may also include a skirt 218 coupled to the prosthetic leaflets 206 and to the frame 202. In an embodiment, as shown in FIG. 5, the skirt 218 is disposed adjacent the inflow end 208 of the transcatheter heart valve prosthesis 200.

The frame 202 of the transcatheter heart valve prosthesis 200 further includes a plurality of struts 212 that are arranged to form a plurality of side openings or cells 214 arranged circumferentially around a longitudinal axis LA of the transcatheter heart valve prosthesis 200 and longitudinally to form a tubular structure defining a central lumen of the transcatheter heart valve prosthesis 200. The struts 212 are defined herein as the elongated wire segments of the frame 202. Struts 212 come together to form crowns or nodes 207. At the outflow end 210 of frame 202, the frame 202 includes tabs 220 extending from two of the crowns. In the embodiment shown, there are two tabs 220 disposed 180 degrees apart from one another around the circumference of the frame 202. However, this is not meant to be limiting, and there can be more or fewer tabs 220 distributed about the frame 202 as desired. As will be explained in more detail below, the tabs 220 are used to couple the frame 202 to the delivery system 300. The tabs 220 may also be radiopaque to assist in visualizing the frame 202 during delivery and deployment of the transcatheter heart valve prosthesis 200.

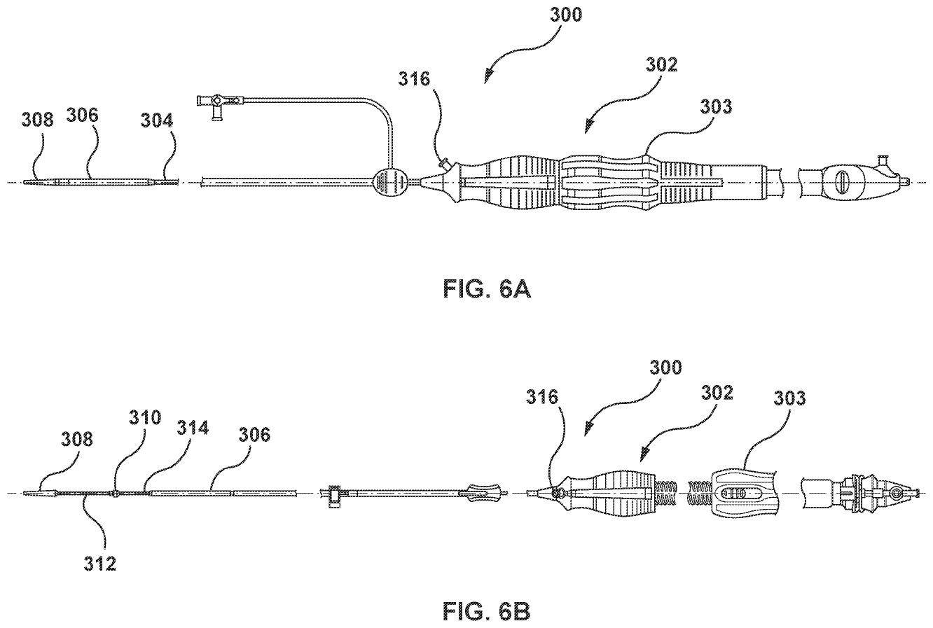

FIGS. 6A and 6B show the example delivery system 300 for a self-expanding transcatheter heart valve prosthesis such as the transcatheter heart valve prosthesis 200. As noted above, the delivery system is merely an example delivery system that can be used with the loading system 100 described above. Further, one skilled in the art will realize that FIGS. 6A and 6B illustrate one example of a delivery system and that existing components illustrated in FIGS. 6A and 6B may be removed and/or additional components may be added to the delivery system 300.

The delivery system 300 includes a handle 302. The handle 302 enables a clinician to manipulate a distal portion of the delivery system 300 and includes actuators for moving parts of the delivery system relative to other parts, such as an actuator 303 for moving an outer shaft 304 relative to an inner shaft 312. A distal portion of the outer shaft 304, referred to as a capsule 306, is configured to surround a transcatheter heart valve prosthesis during delivery to the treatment site, e.g., a native heart valve and is retracted from the transcatheter heart valve prosthesis to expose the transcatheter heart valve prosthesis such that it self-expands. The inner shaft 312 is coupled to the handle 302 and movement of the handle translates to movement of the inner shaft 312 and a distal tip 308 coupled to a distal end of the inner shaft 312. The inner shaft 312 and distal tip 308 may also be translated relative to the outer shaft 304 and the handle 302 via a tip retractor. In the embodiment shown, a middle member 314 is disposed between the inner shaft 312 and the outer shaft 304, and the middle member 314 includes a retainer or spindle 310 attached to a distal portion thereof for receiving the tabs 220 of the transcatheter heart valve prosthesis 200.

In the embodiment shown, a flush port 316 is disposed on the handle 302. In the delivery system 300 shown, when the transcatheter heart valve prosthesis 200 is properly loaded into the delivery system 300, certain relationships between features or the transcatheter heart valve prosthesis 200 and features of the delivery system 300 are present, which may assist in predicting the proper rotational orientation of the transcatheter heart valve prosthesis 200. In particular, when loading the transcatheter heart valve prosthesis 200 into the delivery system 300, as described in more detail below, the tabs 220 are placed into tab pockets 318 of the spindle 310 at 180° apart from each other, as shown in FIG. 8R described below. One of the tabs 220 may include a C-shaped marker (sometimes referred to as a “C-tab” or “C-paddle”) and may be aligned with one of the commissures 216 of prosthetic valve 204. Further, when the transcatheter heart valve prosthesis 200 is loaded into the delivery system, the C-tab 220 is located in the tab pocket 318 that is aligned with the flush port 316 on the handle 302 of the delivery system 300.

As noted above, this is a brief description of an example delivery system 300. Other parts shown in FIGS. 6A and 6B are not described in detail herein and would be apparent to those skilled in the art.

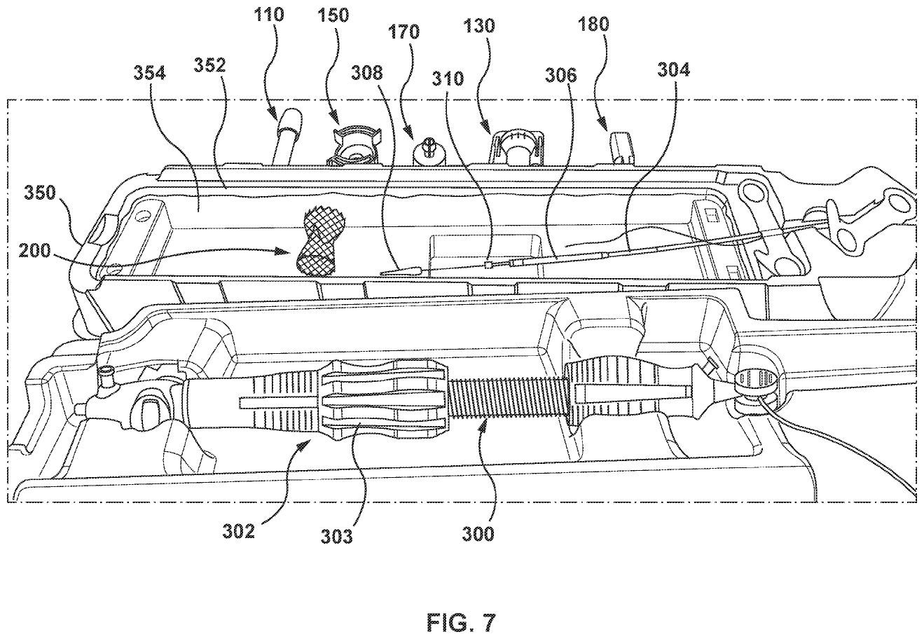

Having described the loading system 100, an example transcatheter heart valve prosthesis 200, and an example delivery system 300, a method of loading the transcatheter heart valve prosthesis 200 into the delivery system 300 using the loading system 100 will now be described with respect to FIG. 7 and FIGS. 8A through 8Y. While FIG. 7 and FIGS. 8A-8Y illustrate various operations that can be performed in the method, one skilled in the art will realize that existing operations can be removed, and additional operations can be added. Likewise, one skilled in the art will realize that the order of the operations can be changed in some instances.

FIG. 7 shows a storage and loading tray 350 that can be used to ship and store the delivery system 300 and to load the transcatheter heart valve prosthesis 200 into the delivery system 300. Example storage and loading trays are described in U.S. Pat. Nos. 8,584,849 and 9,486,604, which are incorporated by reference herein in their entirety. For the purposes of the method described herein, the storage and loading tray 350 includes reservoir 352 filled with a liquid 354, such as chilled saline, for use in loading the transcatheter heart valve prosthesis 200 into the delivery system 300.

As can be seen in FIG. 7, the delivery system 300 is disposed in the loading tray 350 with the handle 302 of the delivery system 300 disposed in a first portion of the loading tray 350 and a distal portion of the delivery system 300, including the capsule 306 and the tip 308, disposed in the reservoir 352 of the loading tray 350. As also can be seen in FIG. 7, the actuator 303 of the delivery system 300 is retracted such that the capsule 306 of the delivery system 300 is retracted from the tip 308, thereby exposing the spindle 310. As can also be seen in FIG. 7, the parts of the loading system 100, including the capsule guide tube 110, the loading cone 130, a loading ring 150, the valve seat 170, and the tip guide tube 180, are separated from each other in preparation for use. Further, the transcatheter heart valve prosthesis 200 is sitting in the cold saline bath in the reservoir 352 to make the frame 202 thereof more pliable for radial compression by the loading system 100. It is noted that the orientation of the loading system 100 shown in FIGS. 8A-8X is consistent with FIG. 1A, but opposite that shown in FIG. 1B.

In a step of the method, the capsule guide tube 110 is slid over the distal tip 308 of the delivery system 300 and moved proximally (see arrow in FIG. 8A) until the second (distal) end 112 of the capsule guide tube 110 is adjacent the distal end of the capsule 306, as shown in FIGS. 8A and 8B. With the capsule guide tube 110 in place, the locking member 116 of the capsule guide tube 110 is moved distally (see arrow in FIG. 8B) to apply an Inward force to the tube 111 of the capsule guide tube 110, which in turn applies a radially inward force to the capsule 306 of the delivery system 300, thereby securing the capsule guide tube 110 to the capsule 306 via friction, as shown in FIG. 8C.

In another step of the method, the loading ring 150 and the valve seat 170 are coupled to each other by inserting the aperture wall 174 of the valve seat 170 into the central passageway 155 of the loading ring 150, as shown by the arrow in FIG. 8D. As noted above, the aperture wall 174 of the valve seat 170 is inserted until the lip 176 of the aperture wall 174 extends past the wall 156 defining the central passageway 155 to secure the valve seat 170 to the loading ring 150. As noted above, with the valve seat 170 and the loading ring 150 secured to each other, the valve seat 170 and the loading ring 150 are rotatable relative to each other. FIG. 8E shows the valve seat 170 and the loading ring 150 secured to each other. Those skilled in the art would understand that the valve seat 170 and the loading ring 150 may be secured to each other before or after the capsule guide tube 110 is secured to the capsule 306.

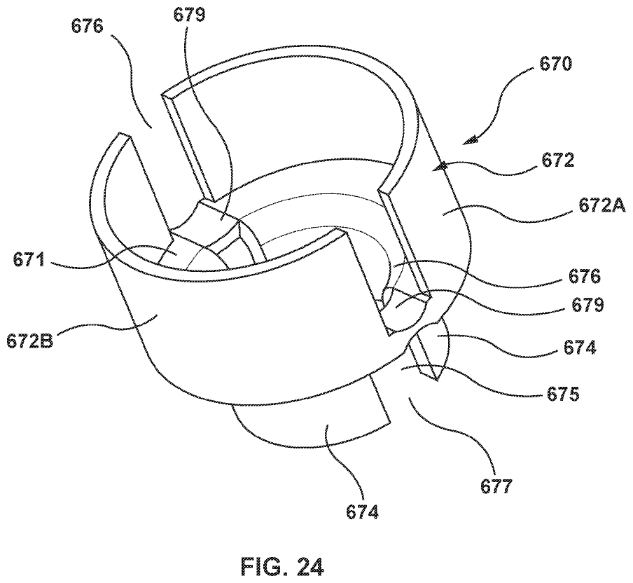

In another step of the method, the transcatheter heart valve prosthesis 200 is loaded into the valve seat 170 that is rotatably secured to the loading ring 150, as shown in FIGS. 8F and 8G. As explained above, the order of operations of the method described is not meant to be limiting. Therefore, for example, the transcatheter heart valve prosthesis 200 may be loaded into the valve seat 170 prior to the valve seat 170 being coupled to the loading ring 150. In the embodiment shown, the transcatheter heart valve prosthesis 200 is loaded into the valve seat 170 with the inflow end 208 of the transcatheter heart valve prosthesis 200 in contact with the base 171 of the valve seat 170. Further, the transcatheter heart valve prosthesis 200 may be slightly radially compressed by hand such that the inflow portion of the heart valve prosthesis is disposed radially inward of the wall 172 of the valve seat 170. The transcatheter heart valve prosthesis 200 and the valve seat 170 combination is rotatable relative to the loading ring 150, as shown by the arrow in FIG. 8F. If the valve seat 670 shown in FIGS. 24-26 is used, the wall sections 672A, 672B are splayed apart prior to inserting the transcatheter heart valve prosthesis 200 into the valve seat 670, thereby eliminating the hand compression described above.

In another step of the method, the loading cone 130 and the loading ring 150 with the valve seat 170 secured thereto and the transcatheter heart valve prosthesis 200 disposed in the valve seat 170 are secured to each other, as shown in FIGS. 8H and 8I. In particular, the first end 131 of the loading cone 130, i.e., the larger diameter end, is slid over the transcatheter heart valve prosthesis 200 and towards the loading ring 150 until the arms 137 of the loading cone 130 extend through the respective slots 157 such that the lip 141 of each of the arms 137 extends past the outer wall 159 of the respective slot 157 and expands radially outward to couple the loading cone 130 to the loading ring 150. Those skilled in the art would recognize that the description above regarding sliding the loading cone 130 towards the loading ring 150 is not meant to be limiting, and relative movement between the two is all that is needed, such that that loading ring 150 can be moved towards the loading cone 130, and/or both the loading ring 150 and the loading cone 130 may be moved towards each other. As the loading cone 130 and the loading ring 150 are secured to each other, the outflow portion of the transcatheter heart valve prosthesis 200 is radially compressed by the tapered portion 135 of the loading cone 130. When the loading cone 130 and the loading ring 150 are secured to each other, the outflow end 210 of the transcatheter heart valve prosthesis protrudes from the second end 132 (smaller diameter end) of the loading cone 130. With the loading cone 130 and the loading ring 150 secured to each other and the transcatheter heart valve prosthesis 200 disposed within the loading cone 130 and the loading ring 150, the rotational orientation of the heart valve prosthesis 200 can be adjusted by rotating the loading cone 130 relative to the loading ring 150, which correspondingly rotates the heart valve prosthesis 200 and the valve seat 170 relative to the loading ring 150.

In another step of the method, the tip guide tube 180 is inserted through the loading ring 150, the valve seat 170, the transcatheter heart valve prosthesis 200, and the loading cone 130, as shown in FIGS. 8J-8L. In particular, in this embodiment, with the loading cone 130 and the loading ring 150 secured to each other, the second end 188 of the tip guide tube 180 is inserted into the central passageway 175 of the valve seat 170, which is disposed within the central passageway 155 of the loading ring 150, as shown in FIG. 8I. The second end 188 of the tip guide tube 180 continues to be advanced through the central passageway 175 of the valve seat 170, through the central lumen of the transcatheter heart valve prosthesis 200 which is disposed within the loading cone 130, until the second end 188 of the tip guide tube 180 protrudes past the second end 132 of the loading cone 130, as shown in FIGS. 8K and 8L. As shown in FIG. 8L, the second end 188 of the tip guide tube 180 prevents the outflow end 210 of the transcatheter heart valve prosthesis 200 from collapsing radially inward. In other words, the second end 188 of the tip guide tube 180 maintains the outflow end 210 of the transcatheter heart valve prosthesis 200 at an inner diameter substantially equal to the outer diameter of the tube 182 of the tip guide tube 180 at the second end 188. This eases coupling of the tabs 220 to the tab pockets 318 of the delivery system 300.

In a next step of the method, the combination of the loading cone 130 coupled to the loading ring 150 with the transcatheter heart valve prosthesis 200 disposed therein on the valve seat 170, with the tip guide tube 180 disposed therethrough, is loaded onto the delivery system 300. In particular, the distal tip 308 of the delivery system 300 is inserted into the lumen 181 at the second end 188 of the tip guide tube 180, as shown in FIG. 8M. The loading system 100 is advanced towards the capsule 306 of the delivery system 300 until the second end 188 of the tip guide tube 180 is adjacent the spindle 310, as shown in FIG. 8N.

In a next step of the method, the tabs 220 are placed in the tab pockets 318 of the spindle 310. In particular, the loading cone 130 is rotated as necessary to rotate the transcatheter heart valve prosthesis 200 as necessary to align the tabs 220 with the tab pockets 318. Further, the tip guide tube 180 is withdrawn slightly as the loading cone 130/loading ring 150 is slightly advanced, and the tabs 220 are placed in the tab pockets 318, as shown in FIGS. 8O and 8P.

In a next step of the method, the tip guide tube 180 is completely removed from the loading cone 130/loading ring 150, as shown in FIG. 8Q. The transcatheter heart valve prosthesis 200 is checked to ensure that the tabs 220 are in the tab pockets 318 and the crowns at the outflow end 210 are properly situated at the spindle 310, as shown in FIG. 8R. FIG. 8S shows the transcatheter heart valve prosthesis 200 with the tabs 220 in the tab pockets 318 of the spindle 310, with the capsule 306 proximal of the spindle 310 and the loading cone 130, the loading ring 150, and the remainder of the inflow end 208 of the transcatheter heart valve prosthesis 200 distal of the spindle 310.

In a next step of the method, the actuator 303 is rotated to advance the capsule distally, i.e., towards the distal tip 308 of the delivery system. In particular, as shown in FIGS. 8T and 8U, the actuator 303 of the delivery system 300 is rotated as shown by the rotational arrow, which causes the capsule 306 of the delivery system 300 to translate distally, as shown by the arrows in FIGS. 8T and 8U. As the capsule 306 is advanced distally, the capsule 306 first covers the tabs 220 of the transcatheter heart valve prosthesis 200 in the tab pockets 318. As the capsule 306 continues to be advanced, the capsule 306 and capsule guide tube 110 push the loading cone 130, loading ring 150, and valve seat 170 distally. Because the transcatheter heart valve prosthesis 200 is prevented from translating distally by the tabs 220 disposed in the tab pockets 318 of the spindle 310, the loading cone 130 moves relative to the transcatheter heart valve prosthesis 200 such that the loading cone 130 radially compresses the transcatheter heart valve prosthesis 200. Further, as the loading cone 130 moves relative to the transcatheter heart valve prosthesis 200, the transcatheter heart valve prosthesis 200 exits the second end 132 (i.e., the smaller diameter end) and enters the open distal end of the capsule 306. This actuation of the actuator 303, distal advancement of the capsule 306, distal translation of the loading cone 130, radial compression of the transcatheter heart valve prosthesis 200, and capture of the transcatheter heart valve prosthesis 200 within the capsule 306 continues until the transcatheter heart valve prosthesis 200 is fully captured within the capsule 306, as shown in FIG. 8V. The combination of the loading cone 130, the loading ring 150, and the valve seat 170 can then be removed from the distal tip 308 of the delivery system 300, as shown in FIG. 8W.

In another step of the method, the capsule guide tube 110 can be unlocked and removed from the capsule 306 and distally over the distal tip 308 of the delivery system 300, as shown in FIG. 8X. In another step of the method, the actuator 303 is rotated, causing the capsule 306 to translate distally until the distal end of the capsule 306 abuts a proximal portion of the distal tip 308, as shown in FIG. 8Y. The delivery system 300 with the transcatheter heart valve prosthesis 200 loaded into the capsule 306 is ready to be used to transluminally deliver the transcatheter heart valve prosthesis 200 to the site of a native heart valve, and deploy the transcatheter heart valve prosthesis 200 from the delivery system 300.

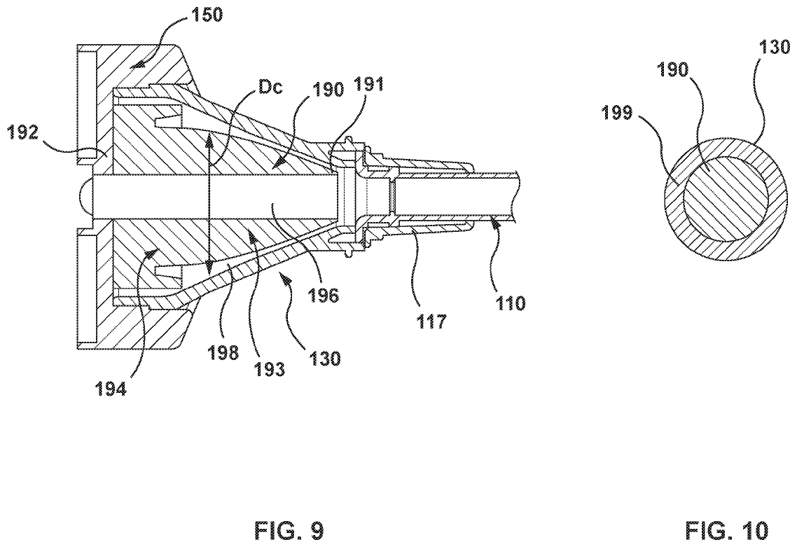

FIGS. 9 and 10 show the loading system 100 with an inner loading cone support 190. One skilled in the art will realize that FIGS. 9 and 10 illustrate one example of an inner loading cone support and that components illustrated in FIGS. 9 and 10 may be removed and/or additional components may be added to the inner loading cone support 190. The inner loading cone support 190 includes a first end 191 and a second end 192 opposite the first end 191. The first end 192 has a first lateral dimension, e.g., a first diameter, which is smaller than a second lateral dimension, e.g., a second diameter, at the second end 192 of the inner loading cone support 192. The inner loading cone support 190 includes a first portion 193 disposed adjacent the first end 191 and a second portion 194 disposed adjacent the second end 192. The first portion 193 includes a tapered outer surface 198 that tapers towards the first end 191 of the inner loading cone support 190.

The inner loading cone support 190 further includes a lumen 196 extending from the first end 191 to the second end 192. The lumen 196 is sized to receive the tube 182 of the tip guide tube 180 therein.

In use, the inner loading cone support 190 is disposed within the loading cone 130 such that the first end 191 of the inner loading cone support 190 is disposed adjacent the second end 132 of the loading cone 130 and the second end 192 of the inner loading cone support 190 is disposed adjacent the first end 131 of the loading cone 130. An annular lumen 199 is defined between the outer surface of the inner loading cone support 190 and the inner surface of the loading cone 130, as shown in FIG. 10. The annular lumen 199 is configured to receive the heart valve prosthesis 200 (not shown in FIGS. 9 and 10).

The inner loading cone support 190 provides an inner profiled surface, i.e., the outer surface of the inner cone support 190, to create a more linear compression of the prosthetic valve 204 of the transcatheter heart valve prosthesis 200 than use of the loading system 100 with the loading cone 130 without the inner loading cone support 190 disposed within the loading cone 130. In other words, the inclusion of the inner loading cone support 190 avoids a more sudden compression near the second end 132 (the smaller diameter end) of the loading cone 130. FIG. 9 shows a threshold diameter D_{C} where the cross-sectional area of the transcatheter heart valve prosthesis 200 (e.g., the frame 202 and prosthetic valve 204) exceeds the cross-sectional area of the annular lumen 199. With an empty loading cone 130 (i.e., without the inner loading cone support 190), the location of the threshold diameter is closer to the second (narrow) end 132 of the loading cone 130. As explained above, this may create a rapid compression of the transcatheter heart valve prosthesis 200 and potentially increase stress on the prosthetic valve 204.

Using the inner loading cone support 190, compression of the prosthetic valve 104 can begin immediately once the transcatheter heart valve prosthesis 200 is loaded into the loading cone 130, even while the transcatheter heart valve prosthesis 200 is static, i.e., before it is pulled through the loading cone 130. As the transcatheter heart valve prosthesis 200 is pulled through the loading cone 130 with the inner loading cone support 190, the annular lumen 199 can be defined and used to control the rate of valve tissue compression, avoiding sudden changes or stress on the tissue of the prosthetic valve 204.

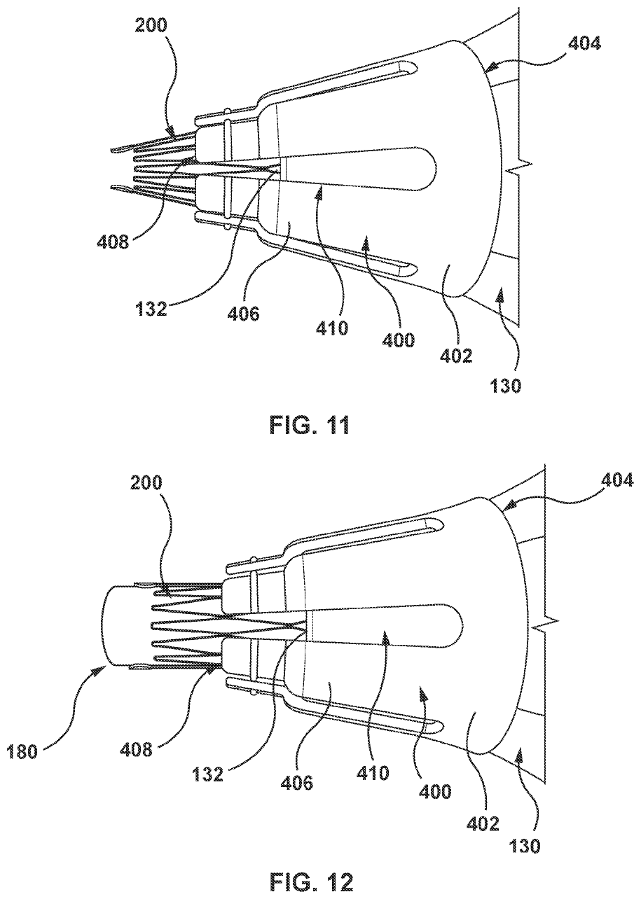

In embodiments, the loading system 100 may include a compliant exit 400, as shown in FIGS. 11-13. The compliant exit 400 assists in loading the transcatheter heart valve prosthesis 200 into the delivery system. 300. In particular, the compliant exit 400 assists in loading the tabs 220 of the transcatheter heart valve prosthesis 200 into the tab pockets 318 of the spindle 310 of the delivery system 300 of the One skilled in the art will realize that FIGS. 11-13 illustrate one example of a compliant exit 400 and that components illustrated in FIGS. 11-13 may be removed and/or additional components may be added to the compliant exit 400.

The compliant exit 400 includes an annular base 402 at a first end 404 of the compliant exit 400, with fingers 406 extending from the base 402 towards a second end 408 of the compliant exit 400. The fingers 406 are separate by gaps 410 around a circumference of the compliant exit 400. The compliant exit 400 is configured to be coupled to the second (narrow) end 132 of the loading cone 130, as shown in FIGS. 11-13. The compliant exit 400 follows the taper of the loading cone 130, with the fingers 406 of the compliant exit 400 extending past the second end 132 of the loading cone 130. The fingers 406 of the compliant exit 400 are more flexible than the loading cone 130 and therefore can flex radially outward as the outflow end 210 of the transcatheter heart valve prosthesis 200 exits the second end 132 of the loading cone 130. This enables the tabs 220 of the transcatheter heart valve prosthesis 200 to be more easily loaded into the tab pockets 318 of the spindle 310 of the delivery system 300 as compared to the second end 132 of the loading cone 130 located at the same location as the second end 408 of the compliant exit 400. A less flexible second end 132 of the loading cone 130 may force the outflow end 210 of the transcatheter heart valve prosthesis 200 radially inwardly such that the tabs 220 need to be manipulated to be placed in the tab pockets 318. Use of the compliant exit 400 enables the tabs 220 to be more radially outward, assisting in placing the tabs 220 into the tab pockets 318.

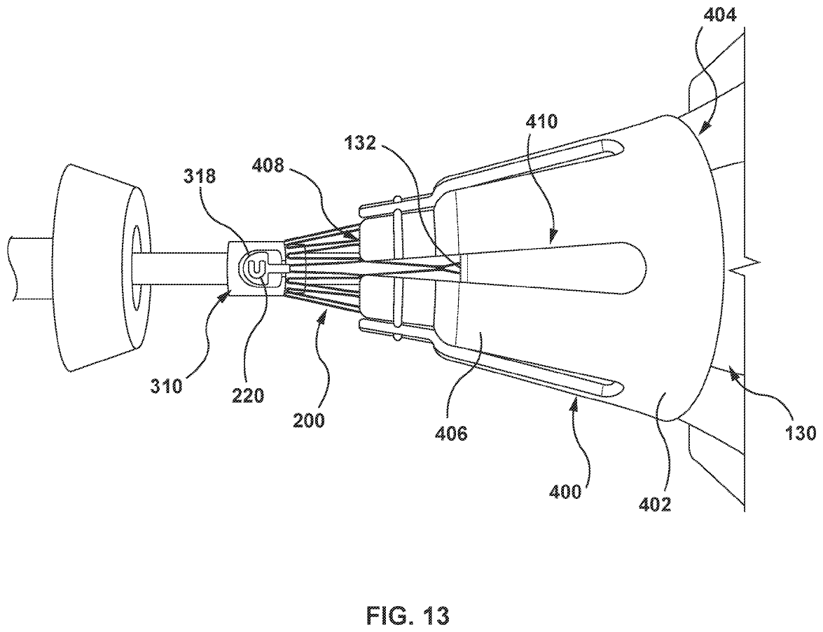

FIG. 11 shows the compliant exit 400 with the outflow end 210 of the transcatheter heart valve prosthesis 200 prior to the tip guide tube 180 being disposed through the loading cone 130. FIG. 12 shows the compliant exit 400 with the tip guide tube 180 disposed through the loading cone 130 and the transcatheter heart valve prosthesis 200. FIG. 13 shows the compliant exit 400 after the tip guide tube 180 has been withdrawn, and with the tabs 220 of the transcatheter heart valve prosthesis 200 inserted into the tab pockets 318 of the spindle 310.

Those skilled in the art would recognize that although the compliant exit 400 is shown as described as a separate item coupled to the loading cone 130, it could instead be part of the loading cone 130. For example, the second end 132 of the loading cone 130 may include fingers such as fingers 406, or may otherwise be made more flexible or compliant to assist in loading the tabs 220 into the tab pockets 318.

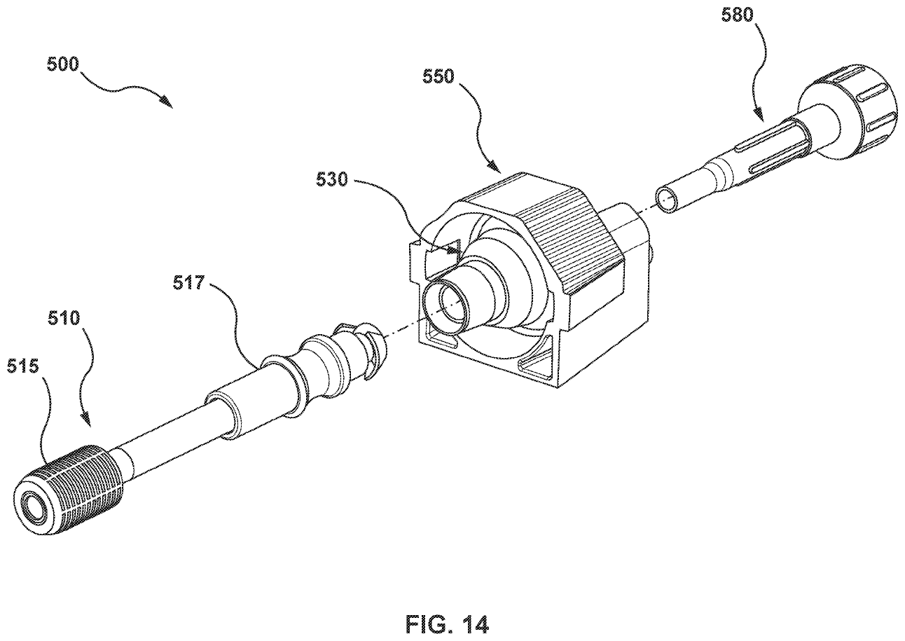

FIGS. 14-23 show another embodiment of a loading system 500 according to embodiments hereof. Like the embodiment of FIGS. 1-4C, the loading system 500 includes a capsule guide tube 510, a loading cone 530, and loading ring 550, and valve seat 570, and a tip guide tube 580. One skilled in the art will realize that FIGS. 14-23 illustrate one example of a loading system and that existing components illustrated in FIGS. 14-23 may be removed and/or additional components may be added to the loading system 500. Further, the parts and features of the loading system 500 are interchangeable with the like parts and features of the loading system 100 described above with respect to FIGS. 1-4C described above. For example, and not by way of limitation, the tip guide tube 580 and valve seat 570 described with respect to the loading system 500 can be used with the remainder of the loading system 100. Also, as the parts of the loading system 500 are similar to the like numbered parts of the loading system 100, description of the loading system 500 will focus on differences between the loading system 500 and the loading system 100.

As explained above, the capsule guide tube 510 is configured to be disposed on a capsule of a delivery system. As shown in FIGS. 14, 15, and 16A-16C, the capsule guide tube 510 includes a tube 511 having a first (distal) end 512, a second (proximal) end 513, and a passageway or lumen 514 extending from the first end 512 to the second end 513. The capsule guide tube 510 further includes a grip 515 coupled to an exterior of the tube 511, and a locking member 516 configured to lock the capsule guide tube 510 to the capsule of the delivery system, as explained above. The locking member 516 is disposed around the tube 511 and includes a lock grip 517 and a lock tube 518.

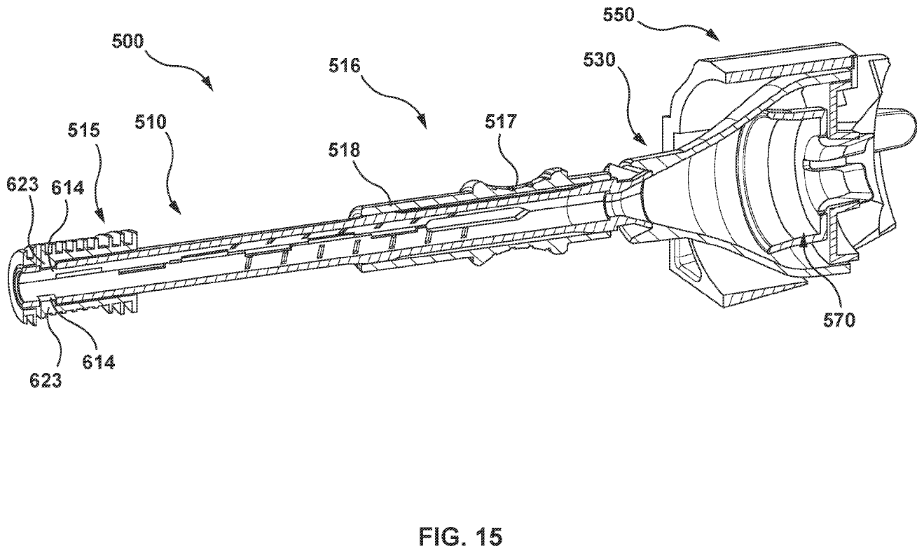

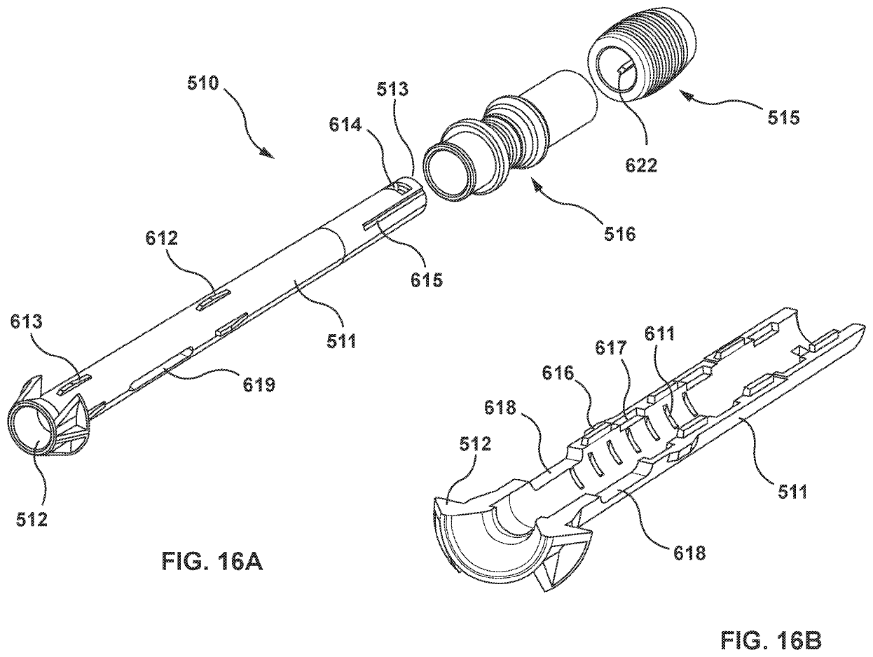

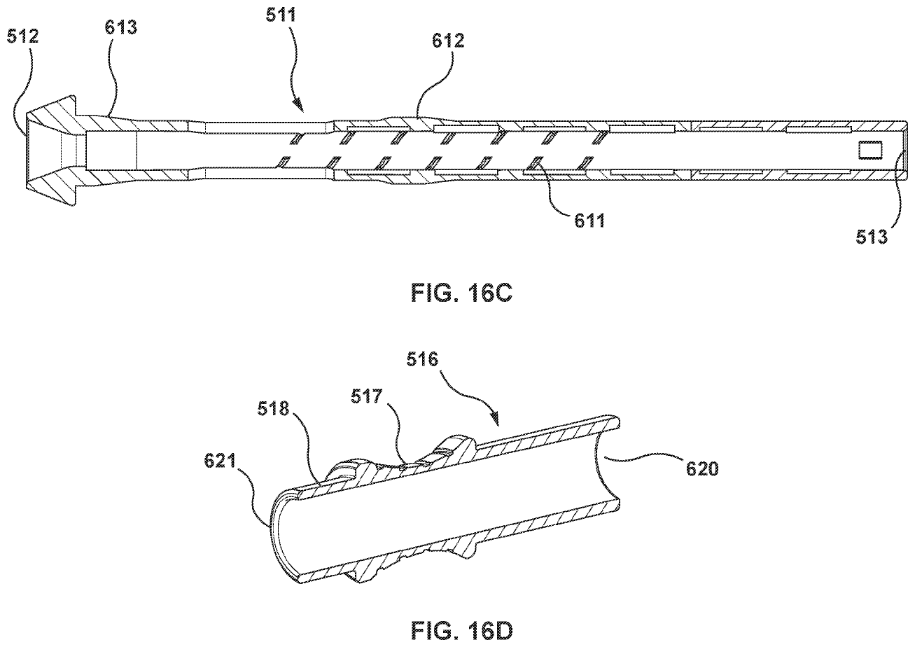

FIGS. 16A-16C show some additional features of the capsule guide tube 510, not described above with respect to the capsule guide tube 110. These features may also be added to the capsule guide tube 110, as appropriate. In one example, internal ribs 611 protrude from an inner surface the tube 511, as shown in FIGS. 15 and 16B. The internal ribs 611 are spaced longitudinally from each other and individually extend circumferentially, as shown in FIGS. 15 and 16B, or angled (as in a spiral), as shown in FIG. 16C. The internal ribs 611 distribute forces during loading.

In another example, the capsule guide tube 510 includes a proximal bump 612 and a distal bump 613, as shown in FIGS. 16A and 16C. The proximal and distal bumps 612, 613 are used in conjunction with the locking member 516 to lock the locking member 516 in place at the distal end of the tube 511 in use, as described above. The embodiment shown includes four proximal bumps 612 distributed equally around the circumference of the tube 511 and four distal bumps 613 distributed equally around the circumference of the tube 511, but this is not meant to be limiting and other arrangements may also be utilized.

The capsule guide tube 510 further includes openings 614 adjacent the second (proximal) end 513 of the tube 511, as shown in FIGS. 15 and 16A. The openings 614 serve as snap fit receivers for corresponding tabs 623 (FIG. 15) extending from an inner surface of the grip 515 to couple the grip 515 to the tube 511. The second (proximal) end 513 of the tube 511 further includes grooves 615 extending longitudinally. The grooves 615 are configured to receive longitudinal extensions 622 extending from the inner surface of the grip 515. The grooves 615 ensure that when coupling the grip 515 to the tube 511, the tabs 623 of the grip 515 are aligned with openings 614 of the tube 511.

As shown in FIG. 16B, the tube 511 of the capsule guide tube 510 can be formed of two halves divided longitudinally. The two parts of the tube 511 may be coupled together via matching tabs 616 and recesses 617. Thus, tabs 616 of one half of the tube 511 align with and are received by corresponding recesses 617 in the other half of the tube 511, as shown in FIG. 16B. Each of the halves of the tube 511 may further include longitudinal depressions 618 that align with corresponding longitudinal depressions 618 in the other half of the tube 511 to form windows 619 when the halves are coupled, as shown in FIG. 16A. The windows 619 enable the tube 511 to flex in the area of the windows 619.

FIG. 16D shows an embodiment of the locking member 516. The locking member 516 includes the lock tube 518 extending from a proximal end 620 to a distal end 621 thereof. The lock grip 517, such as a thumb grip/slider, is formed on an outer surface of the lock tube 518 between the proximal and distal ends 620, 621. In an embodiment, the inner diameter of the lock tube 518 tapers from the proximal end 620 to the distal end 621. In a non-limiting example, the inner diameter at the proximal end 620 is about 10.10 mm and the inner diameter at the distal end 621 is about 9.90 mm. Thus, in the embodiment shown, it is a small taper.

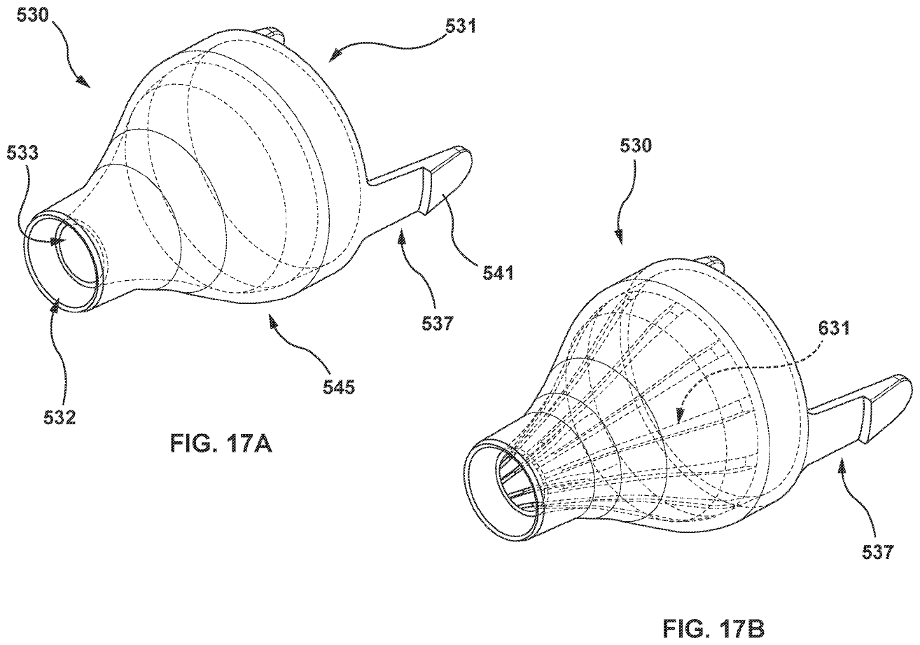

FIGS. 17A and 17B show the loading cone 530 according to embodiments hereof. The loading cone 530 of FIG. 17B includes internal ribs 631 not included in the loading cone 530 of FIG. 17A. The internal ribs 631 may reduce friction. As with the loading cone 130, the loading cone 530 includes a main body 545 including a first (distal) end 531, a second (proximal) end 532, and a central passageway 533 extending from the first end 531 to the second end 532. The internal ribs 631 may extend along the length of the tapered portion 535 of the main body 545. However, this is not meant to be limiting, and in other embodiments, the internal ribs 631 may extend along only a portion of the tapered portion 535.

Similar to the loading cone 130, the loading cone 530 includes locking arms 537 for coupling the loading cone 530 to the loading ring 550, as explained in more detail below. The locking arms 537 of the loading cone 530 extend distally from the first (distal) end 531 of the main body 545. In the embodiment shown, the two locking arms 537 are disposed 180 degrees apart from each other around the circumference of main body 545. Each of the locking arms 537 extends longitudinally from the main body 545 with a distal portion that is tapered from a first thickness adjacent a lip 541 to a second thickness, smaller than the first thickness, at a distal end of the arm 539. In other words, the distal portion of each arm 537 includes a ramp between the lip 541 and the distal end of the arm 537. The locking arms 537 are configured to couple the loading cone 530 with the loading ring 550, as described in more detail below.

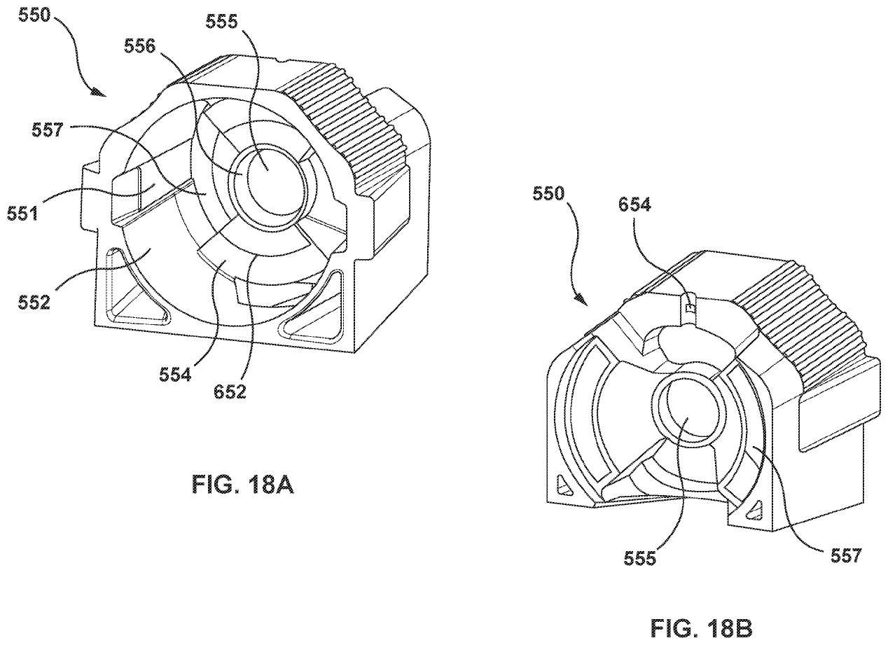

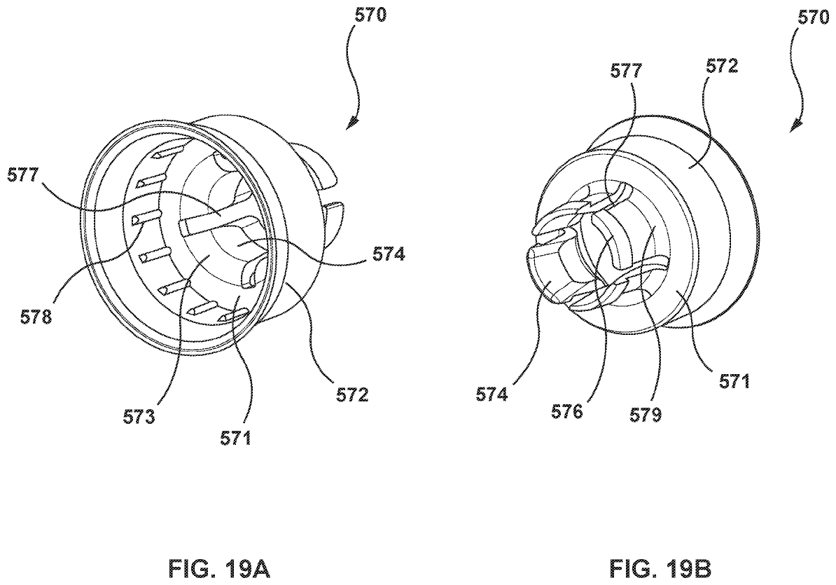

The loading ring 550 of the loading system 500 is shown in FIG. 18A-18B. The loading ring 550 includes a generally cylindrical wall 552, a platform 554 extending inwardly from the wall 552, and a central lumen or passageway 555 extending through the platform 554. A cylindrical wall 556 defines the central passageway 555. Still referring to FIGS. 18A and 18B, the loading ring 550 further two recesses 551 extending longitudinally along the inner surface of the wall 552. The recesses 551 are sized and shaped to receive the locking arms 537 of the loading cone 530. The loading ring 550 further slots 557 disposed at the distal end of the recesses 551, with each slot 557 configured to receive a corresponding locking arm 537 of the loading cone 530. The locking arms 537 extend through the slots 557 and the lips 541 of the locking arms 537 engage a distal surface of the wall 552 to couple the loading cone 530 to the loading ring 550. In the embodiment shown, the slots 557 are sized to enable rotation of the loading cone 530 relative to the loading ring 550, as described above with respect to the loading system 100. However, although the slots 557 are sized to enable rotation, the locking arms 537 are disposed in the recessed 551, thereby preventing rotation. Further, in other embodiments, the slots 557 may be sized such that the loading cone 530 is not rotatable relative to the loading ring 550. Thus, rotation of the transcatheter heart valve prosthesis 200 for alignment may be accomplished by rotation of the valve seat 570, as described below.

The loading ring 550 may further include a rib 652 extending proximally from the platform 554. The rib 652 extends circumferentially around the platform 554 such as to reduce friction between the valve seat 570 and the platform 554 during rotation of the valve seat 570 relative to the loading ring 550. Although a single circumferential rib 652 is shown, this is not meant to be limiting.

The loading ring 550 may further include an opening 654 for creating an air pathway.

FIGS. 19A and 19B show a valve seat 570 according to embodiments hereof. Similar to the valve seat 170 described above, the valve seat 570 includes a base 571 having a central aperture 573 and a wall 572 extending from a periphery of the base 571 in a first direction generally perpendicular to the base 571. The valve seat 570 further includes an aperture wall 574 extending from a periphery of the aperture 573 in a second direction, opposite the first direction, and generally perpendicular to the base 571. A central passageway 575 is defined by the aperture wall 574 and extends in the second direction from the central aperture 573 of the base 571. The aperture wall 574 may include a lip 576 or similar feature to couple the valve seat 570 to the loading ring 550, as described above. The aperture wall 574 may also include a second lip 579 or similar feature adjacent the base 571, as shown in FIG. 19B. The aperture wall 574 of the valve seat 570 is configured to extend through the central passageway 555 the loading ring 550 until the lip 576 of the aperture wall 574 extends past the wall 556 defining the central passageway 555 such that the lip 576 can prevent longitudinal movement of the valve seat 570 with respect to the loading ring 550. However, the valve seat 570 is rotatable relative to the loading ring 550 because the aperture wall 574 is rotatable within the wall 556 of the loading ring 550. If the valve seat 570 and the loading ring 550 need to be separated from each other, the aperture wall 574 of the valve seat 570 may be squeezed radially inward, which may be made easier by slots 577 in the aperture wall 574, to enable the lip 576 to clear the end of the wall 556. The slots 577 in the aperture wall 574 also define four sections of the aperture wall 574. The four wall sections of the aperture wall 574 are configured to be received within recesses of the tip guide tube 580, with ribs of the tip guide tube 580 disposed in the slots 577, as described in more detail below.

In the embodiment of FIG. 19A, the valve seat 570 further includes ribs 578 extending along the wall 572. The ribs 578 extend inwardly and longitudinally from the wall 572 as are distributed around the circumference of the wall 572. The ribs 578 aid in the valve seat engaging the transcatheter heart valve prosthesis 200 such that rotation of the valve seat 570 results in corresponding rotation of the transcatheter heart valve prosthesis 200.

As with the valve seat 170 described above, various sized valve seats 570 may be used such that the same capsule guide tube 510, loading cone 530, loading ring 550, and tip guide tube 580 may be used with various sized transcatheter heart valve prostheses by using different valve seats.

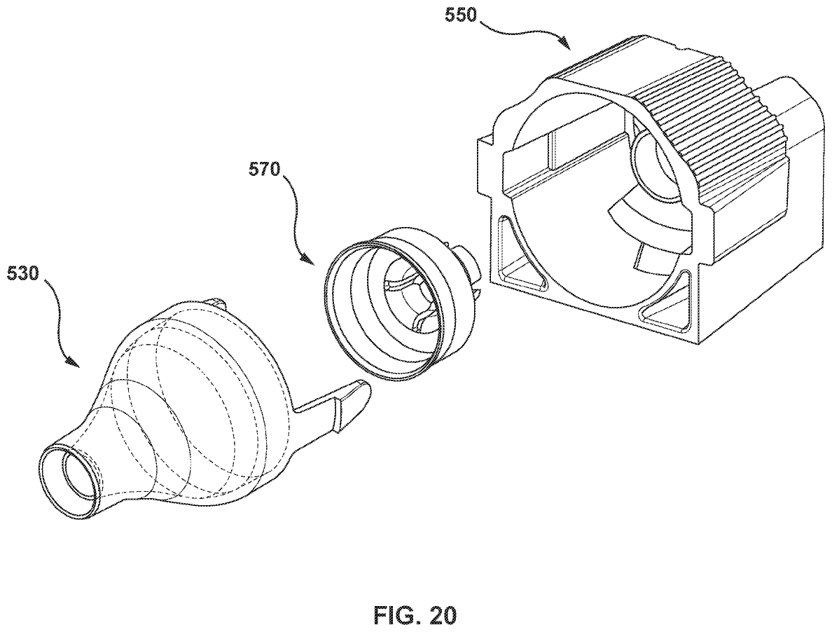

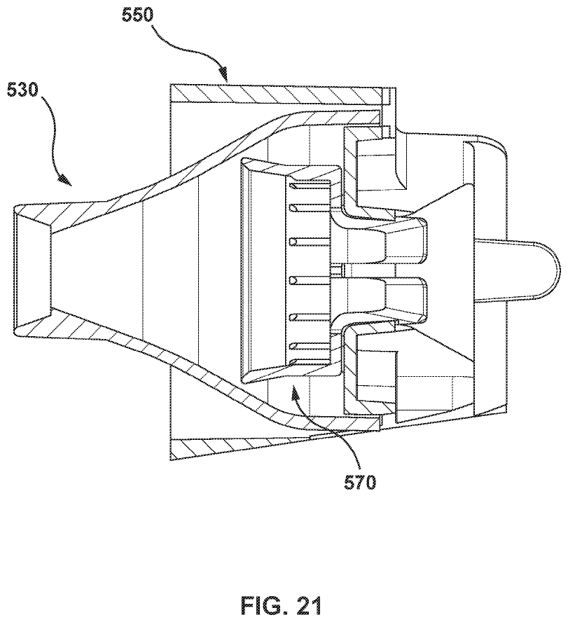

FIG. 20 shows the loading cone 530, the loading ring 550, and the valve seat 570 separated from each other, and FIG. 21 shows the loading cone 530, the loading ring 550, and the valve seat 570 coupled to each other. When used to load a transcatheter heart valve prosthesis 200 into a delivery system, the transcatheter heart valve prosthesis 200 would be disposed on the valve seat 570 within the loading cone 530 and the loading ring 550.

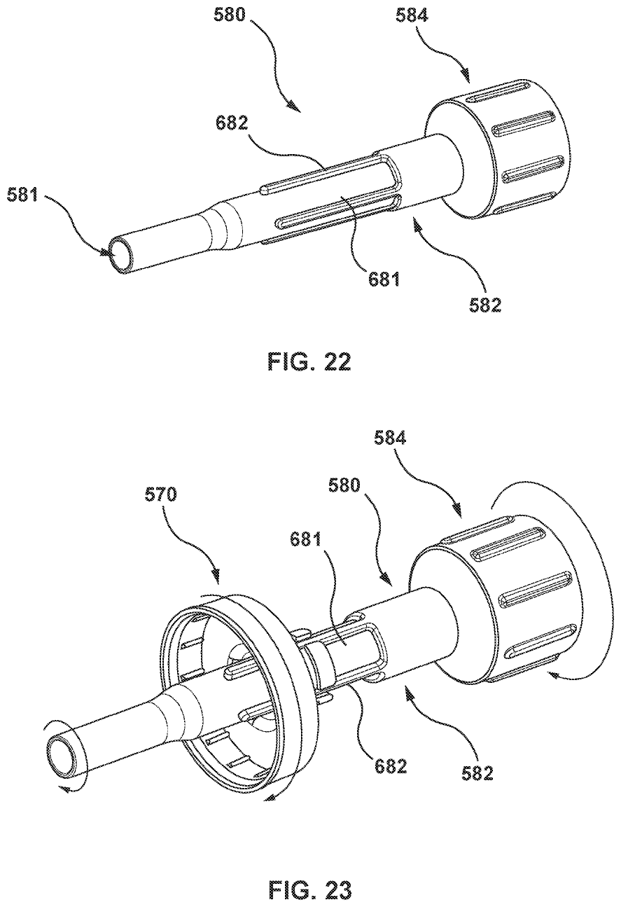

FIG. 22 shows the tip guide tube 580 of the loading system 500. The tip guide tube 580 includes a tube 582 and a grip 584. The tube 582 includes a lumen 581 disposed at least partially therethrough and configured to receive the tip 308 of the delivery system 300, as described above with respect to FIG. 8M. In the embodiment of FIG. 22, the tube 582 includes recesses 681 and ribs 682 alternatingly disposed around an outer surface of the tube 582. In the embodiment shown, there are four recesses 681 and four ribs 682, corresponding to the four portions of the aperture wall 574 and the four slots 577 of the valve seat 570, respectively. The recesses 681 are sized and shaped to receive the four portions of the aperture wall 574, and the four ribs 682 are sized and shaped to extend through the four slots 577, as shown in FIG. 23. However, this is not to be limiting, and more or fewer recesses and ribs may be utilized to match more or fewer wall portions and slots in the valve seat. As further shown in FIG. 23, rotation of the tip guide tube 580 imparts corresponding rotation on the valve seat 570, which can rotate within the loading ring 550. With the transcatheter heart valve prosthesis 200 disposed in the valve seat 570, this rotation also imparts corresponding rotation of the transcatheter heart valve prosthesis 200 to enable alignment of the transcatheter heart valve prosthesis 200 with the delivery system 300, as described above.

The method described above with respect to the loading system 100 is substantially similar to the method used with respect to the loading system 500. However, when the method described above refers to rotation of the transcatheter heart valve prosthesis 200 to align the tabs thereof with the tab pockets, the tip guide tube 580 would be rotated instead of the loading cone 130 described above.

FIGS. 24-26 show a valve seat 670 according to embodiments hereof. FIGS. 24-26 show schematic illustration of the valve seat 670. Those skilled in the art will recognize that other features may be added to the valve seat 670 shown in FIGS. 24-26. For example, and not by way of limitation, the features described with respect to the valve seat 170 and/or the valve seat 570 may be included in the valve seat 670. Similarly, the features described below with respect to the valve seat 670 may be incorporated into the valve seat 170 and/or the valve seat 570. In some embodiments, when manually radially compressing the transcatheter heart valve prosthesis 200 to insert the transcatheter heart valve prosthesis 200 into a valve seat, the transcatheter heart valve prosthesis 200 is over-compressed and then recoils to contact the walls of the valve seat. Such compression and recoil may inadvertently introduce recoverable strain (i.e., pseud-elasticity) into the frame of the transcatheter heart valve prosthesis 200. Therefore, the valve seat 670 includes a living hinge 679 that enables the valve seat 670 to open to receive the transcatheter heart valve prosthesis 200, as described below.

Similar to the valve seats 170, 570 described above, the valve seat 670 includes a base 671 having a central aperture 673 and a wall 672 extending from a periphery of the base 671 in a first direction generally perpendicular to the base 671. In the embodiment shown, the wall 672 includes slots 676 extending in the first direction, thereby separating the wall 672 into two sections. In the embodiment shown, the wall 672 includes two sections 672A, 672B that are approximately semi-circular such that the wall 672 and the slots 676 define a circumference around the base 671. The base 671 further includes a living hinge 679 extending across a diameter of the base 671 from one of the slots 676 to the other of the slots 676, interrupted by the central aperture 673, as shown in FIG. 24. In the embodiment shown, the living hinge 679 is a channel in the first and second surfaces of the base 671, but other arrangements for enabling the walls 672 to spread apart or splay, as described below, may also be used.

The valve seat 670 further includes an aperture wall 674 extending from a periphery of the aperture 673 in a second direction, opposite the first direction, and generally perpendicular to the base 671. A central passageway 675 is defined by the aperture wall 674 and extends in the second direction from the central aperture 673 of the base 671. The aperture wall 674 may include a lip as described with respect to FIGS. 19A-19B or similar feature to couple the valve seat 670 to the loading ring 550, as described above. The aperture wall 674 may also include a second lip or similar feature adjacent the base 671, as shown in FIG. 19B. The aperture wall 674 in the embodiment of FIGS. 24-26 includes two slots 677 separating the wall 674 into two wall sections 674A, 674B, which are approximately semi-circular. The slots 677 enable the wall sections 674A, 674B to be squeezed together to enable the lip (not shown) to clear the end of the wall 556, as described above.

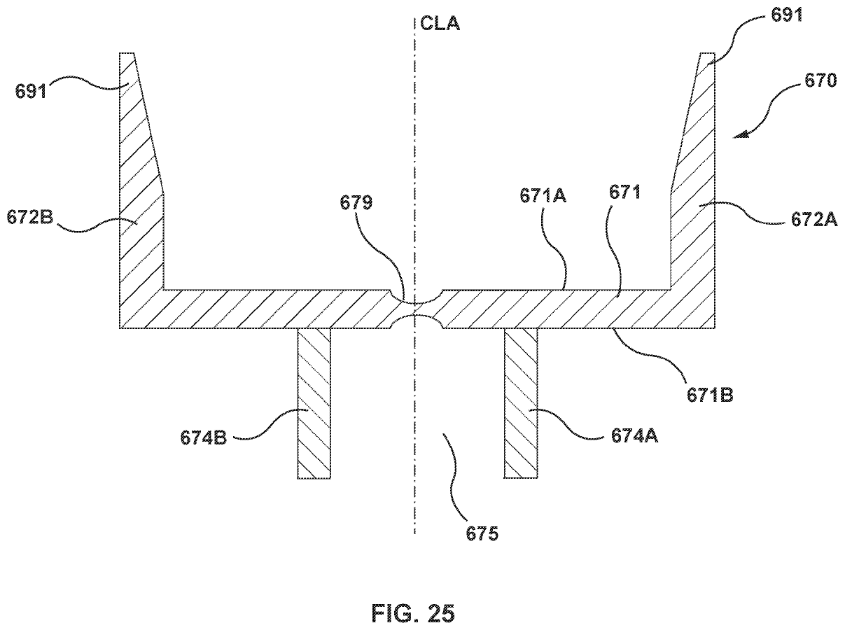

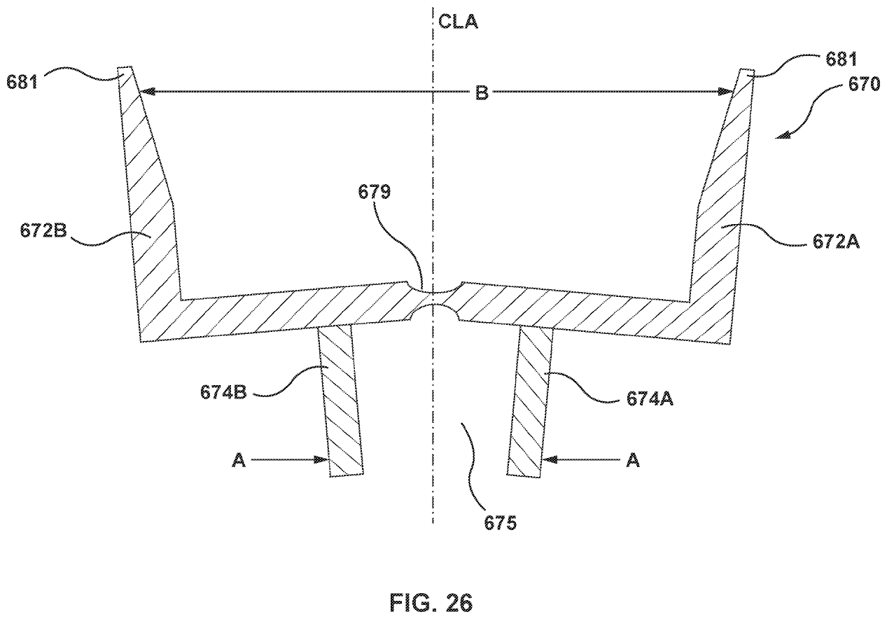

However, in the embodiment of FIGS. 24-26, due to the living hinge 679 and the slots 676 separating the wall 672 into two wall sections 672A, 672B, squeezing the two wall sections 274A, 674B together, as indicated by the arrows A-A in the lower portion of FIG. 26, causes the wall sections 672A, 672B to splay open, as indicated by the arrow B in the upper portion of FIG. 26. In other words, without the wall sections 674A, 674B being squeezed together, the wall sections 574A, 574B and 672A, 672B are substantially parallel to a central longitudinal axis CLA of the valve seat 670, as shown in FIG. 26. When the wall sections 674A, 674B are squeezed towards the central longitudinal axis CLA, end portions 691 of the wall sections 672A, 672B opposite the base 671 splay outwardly relative to the central longitudinal axis CLA, as shown in FIG. 25. Upon release of the radially inward force on wall sections 674A, 674B, the wall sections 674A, 674B and 672A, 672B return to the configuration of FIG. 25.

The outward splaying of the wall sections 672A, 672B enables the transcatheter heart valve prosthesis 200 to be inserted into the valve seat 670 without radially compressing the frame 202 of the transcatheter heart valve prosthesis 200. Thus, the frame 202 is only radially compressed to create a friction fit by the wall sections 672A, 672B when the wall sections 674A, 674B are released, thereby returning the wall sections 672A, 672B to the configuration of FIG. 25. Thus, over-compression and then recoil of the frame 202 are prevented. Instead, the frame 202 is compressed a single time to the proper compression for the friction frit between the transcatheter heart valve prosthesis 200 and the wall 672 of the valve seat 670. Such a single compression results in the highest interference fit force and a repeatable interference fit force between the transcatheter heart valve prosthesis 200 and the wall 672 of the valve seat 670.

Other features described with respect to the valve seats 170, 570 may be included in the valve seat 670 of FIGS. 24-26. In particular, and not by way of limitation, the circumferential ribs 576 and 578 on the wall 574 may be included in the aperture wall 674. Similarly, the ribs 578 shown in FIG. 19A may be included in the wall 672 of the valve seat 670. As with the valve seats 170, 570 described above, various sized valve seats 670 may be used such that the same capsule guide tube 110, 510, loading cone 130, 530, loading ring 150, 550, and tip guide tube 180, 580 may be used with various sized transcatheter heart valve prostheses by using different valve seats.

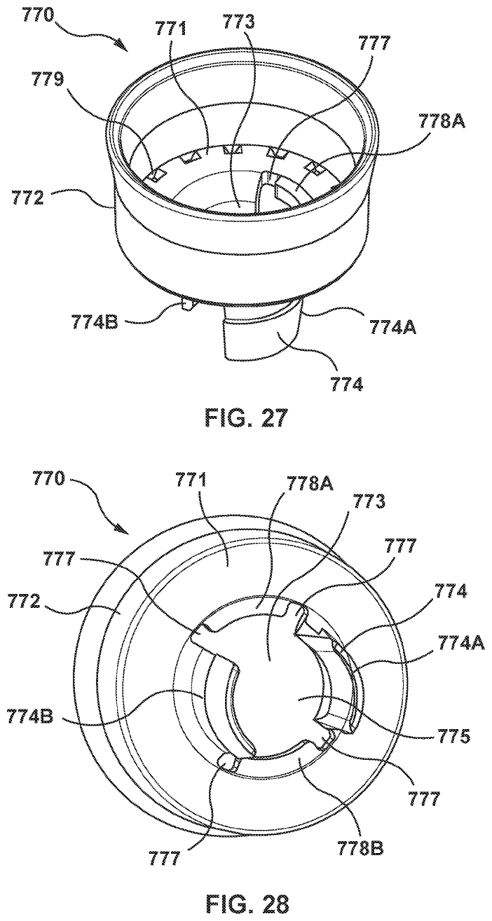

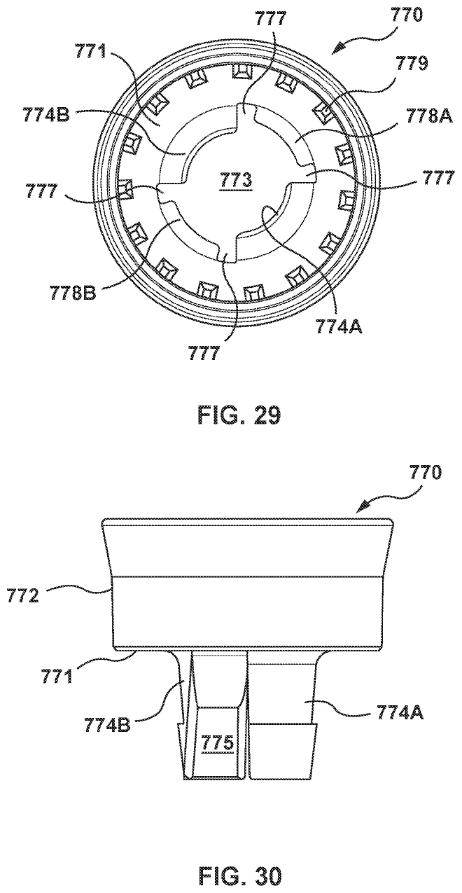

FIGS. 27-30 show a valve seat 770 according to embodiments hereof. FIGS. 27-30 show schematic illustration of the valve seat 770. Those skilled in the art will recognize that other features may be added to the valve seat 770 shown in FIGS. 27-30. For example, and not by way of limitation, the features described with respect to the valve seat 170, 570, and/or 670 may be included in the valve seat 770, where applicable. Similarly, the features described below with respect to the valve seat 770 may be incorporated into the valve seat 170, the valve seat 570, and/or the valve seat 670, where applicable. As described above with respect to the loading system 500, the valve seat is rotated by the tip guide tube 580. When the valve seat is rotated, the transcatheter heart valve prosthesis 200 is also rotated. In some embodiments, it may be desirable to avoid the primary means by which the transcatheter heart valve prosthesis 200 is rotated to be contact between the valve seat and tissue of the transcatheter heart valve prosthesis 200. Therefore, as described below, the valve seat 770 includes grooves or pockets in the base thereof configured to receive crowns of the transcatheter heart valve prosthesis 200.

Similar to the valve seats 170, 570, 670 described above, the valve seat 770 includes a base 771 having a central aperture 773 and a wall 772 extending from a periphery of the base 771 in a first direction generally perpendicular to the base 771. The valve seat 770 further includes an aperture wall 774 extending from a periphery of the aperture 773 in a second direction, opposite the first direction, and generally perpendicular to the base 771.

A central passageway 775 is defined by the aperture wall 774 and extends in the second direction from the central aperture 773 of the base 771. The aperture wall 774 may include a lip as described with respect to FIGS. 19A-19B or similar feature to couple the valve seat 770 to the loading ring 550, as described above. The aperture wall 774 may also include a second lip or similar feature adjacent the base 771, as shown in FIG. 19B. The aperture wall 774 in the embodiment of FIGS. 27-30 includes two wall sections 774A, 774B. However, the valve seat 770 further includes two extensions 778A, 778B extending radially inward from the base 771, with each extension 778A, 778B disposed between the two wall sections 774A, 774B such that the wall sections 774A, 774B and the extensions 778A, 778B alternate around a circumference of the central aperture 773. The valve seat 770 further includes four slots 777 defined between adjacent wall sections 774A, 774B and extensions 778A, 778B. The wall sections 774A, 774B and the extensions 778A, 778B are configured to be received within the recesses 681 of the tip guide tube 580, with the ribs 682 of the tip guide tube 580 disposed in the slots 777, as described above. Although the valve seat 770 has been described as having two wall sections 774A, 774B, two extensions 778A, 778B, and four slots 777, this is not meant to be limiting. In other embodiments, the valve seat may include three or four or more wall sections 774, and more or fewer extensions 778 (including no extensions). Further, the valve seat 770 may include more of fewer slots 777, with the tip guide tube 580 having a corresponding number of ribs 682. It has been found that three of four wall sections 774 provide increases stability during use. Further, the length of the wall sections may be varied. It has been found that longer legs provide easier assembly and disassembly of the valve seat with the loading ring 550.

The valve seat 770 further includes a plurality of grooves, depressions, recesses, or pockets 779 extending into the base 771. The pockets 779 extend into a surface of the base 771 configured to receive the transcatheter heart valve prosthesis 200. In other words, the pockets 779 extend into the surface of the base 771 facing the first direction. The pockets 779 are configured to receive crowns 207 of the frame 202 of the transcatheter heart valve prosthesis 200. In the embodiment shown, the crowns 207 at the inflow end 208 of the frame 202 of the transcatheter heart valve prosthesis 200 are received within the pockets 779. Thus, the quantity of pockets 779 matches the quantity of crowns 207 at the inflow end 208 of the frame 202 of the transcatheter heart valve prosthesis 200. For example, and not by way on limitation, in the embodiment shown, there are fifteen pockets 779 corresponding with fifteen crowns 207 at the inflow end 208 of the frame 202. In the embodiment shown, the pockets 779 do not extend entirely through the base 771 of the valve seat 770, as can be seen in FIG. 28 where the pockets 779 are not seen because it is a bottom isometric view of the valve seat 770. In embodiments, the depth of each pocket 779 is smaller than the length of the crowns 207/struts 212 at the inflow end 208 of the frame 202 that is uncovered by the skirt 218, noted as L1 in FIG. 5. The crowns 207 disposed with the pockets 779 provide the primary force to rotate the transcatheter heart valve prosthesis 200 when the valve seat 770 is rotated, such as by the tip guide tube 580. Due to the primary force for rotating the transcatheter heart valve prosthesis 200 being the uncovered crowns 207, tissue of the transcatheter heart valve prosthesis 200 (e.g., the prosthetic valve leaflets 206 and the skirt 218) are less likely to be damaged than embodiments where a surface surrounding an outer surface of the frame 202 interacts with the outer surface of the frame 202 to rotate the transcatheter heart valve prosthesis 200.

Other features described with respect to the valve seats 170, 570, 670 may be included in the valve seat 770 of FIGS. 27-30. In particular, and not by way of limitation, the circumferential ribs 576 and 578 on the wall 574 may be included in the aperture wall 774. Similarly, the ribs 578 shown in FIG. 19A may be included in the wall 772 of the valve seat 770, although they should not be needed due to the pockets 779. As with the valve seats 170, 570. 670 described above, various sized valve seats 770 may be used such that the same capsule guide tube 110, 510, loading cone 130, 530, loading ring 150, 550, and tip guide tube 180, 580 may be used with various sized transcatheter heart valve prostheses by using different sized valve seats.

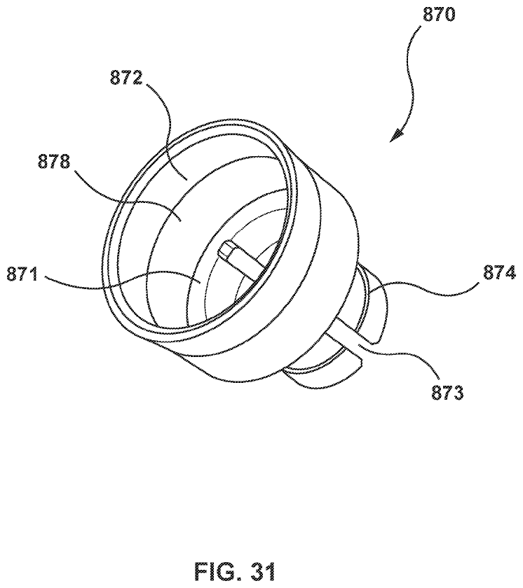

FIG. 31 shows a valve seat 870 according to embodiments hereof. The valve seat 870 shown in FIG. 31 is similar to the valve seats 170, 570, 670, and/or 770 described herein. Therefore, all of the details of the valve seat 870 will not be repeated. Thus, the valve seat 870 generally includes a base 871 having a central aperture 873 and a wall 872 extending from a periphery of the base 871 in a first direction generally perpendicular to the base 871. The valve seat 870 further includes an aperture wall or legs 874 extending from a periphery of the aperture 873 in a second direction, opposite the first direction, and generally perpendicular to the base 871. In the embodiment of FIG. 31, the wall 872 includes a surface treatment 878 disposed at least partially around the interior circumference of the wall 872 and at least partially along the height of the wall 872. The surface treatment 878 is configured to retain the transcatheter heart valve prosthesis 200 such that when the valve seat 870 is rotated, such as by the tip guide tube 570, the transcatheter heart valve prosthesis 200 rotates with the valve seat 870. Thus, the surface treatment 878 acts similarly to the ribs 578 of the valve seat 570 shown in FIG. 19A, and to the pockets 779 of the valve seat 770 shown in FIGS. 27-30. The surface treatment 878 may be various injection molding surface finishes. For example, and not by way of limitations, various matte finishes like SPI B-1 or VDI 24 may be used. The surface treatment 878 is selected such as to slightly increase the friction between the heart valve prosthesis 200 and the valve seat 870 without damaging an inflow wrap/skirt at the inflow portion of the heart valve prosthesis 200. Although FIG. 31 shows the surface treatment 878 disposed around the entire interior circumference of the wall 872 and partially along the interior length of the wall 872. However, this is not meant to be limiting, and the surface treatment 878 more of less of the interior surface of the wall 872. The features of other valve seats described herein may be included in the valve seat 870, where applicable. Similarly, the surface treatment 878 described herein with respect to the valve seat 870 may be included in the other valve seats described herein.

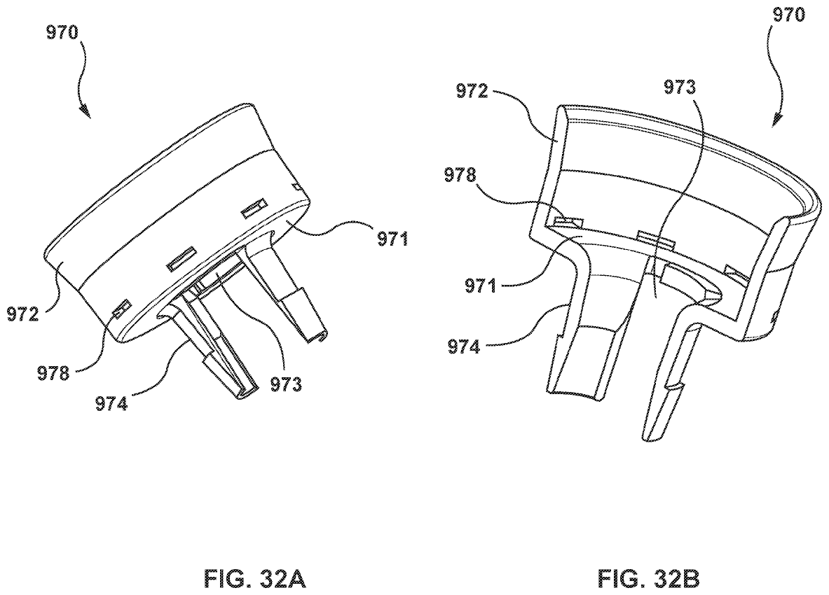

FIGS. 32A and 32B show a valve seat 970 according to embodiments hereof. The valve seat 970 shown in FIGS. 32A and 32B is similar to the other valve seats described herein. Therefore, all of the details of the valve seat 970 will not be repeated. Thus, the valve seat 970 generally includes a base 971 having a central aperture 973 and a wall 972 extending from a periphery of the base 971 in a first direction generally perpendicular to the base 971. The valve seat 970 further includes an aperture wall or legs 974 extending from a periphery of the aperture 973 in a second direction, opposite the first direction, and generally perpendicular to the base 971. In the embodiment of FIGS. 32A and 32B, the wall 972 includes openings 978 extending from an inner surface of the wall 972 to an outer surface of the wall 972. The openings 978 are disposed adjacent to the base 971. The openings 978 enable a user to visualize when the transcatheter heart valve prosthesis 200 has been properly inserted into the valve seat 900. In other words, the openings 978 in the wall 972 adjacent to the base 971 enable a user to see that the crowns 207 at the inflow end 208 of the transcatheter heart valve prosthesis 200 are resting against the base 971. In the embodiment shown in FIGS. 32A and 32B, the wall 972 includes eight openings 978. However, this is not meant to be limiting, and more or fewer openings may be included.

The features of other valve seats described herein may be included in the valve seat 970, where applicable. Similarly, the surface treatment 978 described herein with respect to the valve seat 970 may be included in the other valve seats described herein. For example, and not by way of limitation, the pockets 779 of the valve seat 770 may be included in the valve seat 970. In such a combination, the openings 978 of the valve seat 970 would enable a user to confirm that the crowns 207 at the inflow end 208 of the heart valve prosthesis 200 are inserted into the pocket 779 of the base 970. Similarly, features or other valve seats described herein, such as, but not limited to, the ribs 578 of the valve seat 570 and/or the surface treatment 878 of the valve seat 870, may also be included in the valve seat 970.

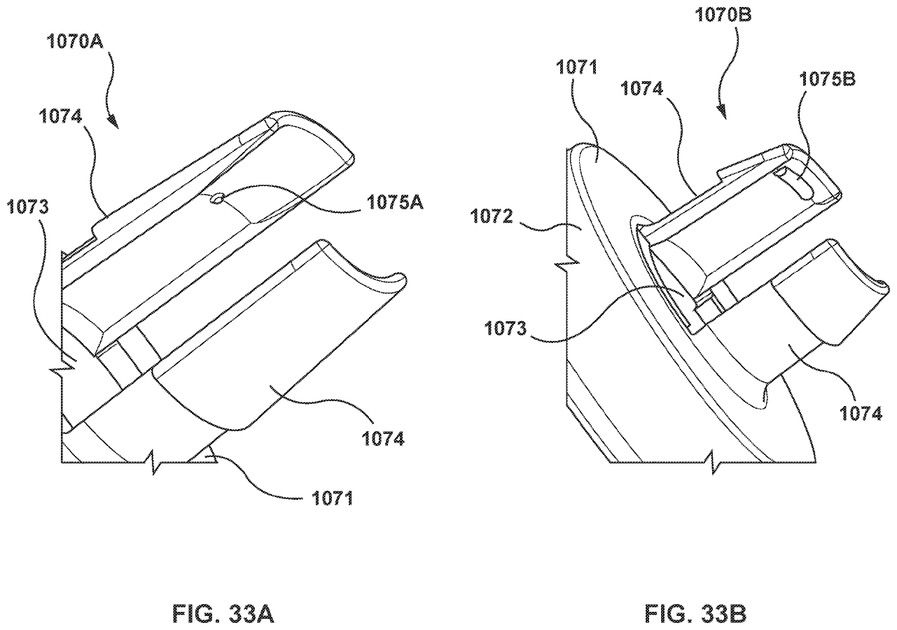

FIGS. 33A and 33B show similar valve seats 1070A and 1070B according to embodiments herein. The valve seats 1070A and 1070B shown in FIGS. 33A and 33B are similar to the other valve seats described herein. Therefore, all of the details of the valve seats 1070A and 1070B will not be repeated. Thus, the valve seats 1070A and 1070B generally include a base 1071 having a central aperture 1073 and a wall 1072 extending from a periphery of the base 1071 in a first direction generally perpendicular to the base 1071. The valve seats 1070A and 1070B further include an aperture wall or legs 1074 extending from a periphery of the aperture 1073 in a second direction, opposite the first direction, and generally perpendicular to the base 1071. In the embodiment of FIGS. 33A and 33B, an interior surface of each leg 1074 includes a protrusion 1075A, 1075B extending toward a central longitudinal axis of the valve seat 1070A, 1070B. In the embodiment of FIG. 33A, the protrusion 1075A is a bump. In the embodiment of FIG. 33B, the protrusion 1075B is a ledge. However, this is not meant to be limiting, and any protrusion may be utilized that is compatible with the purpose described herein. The protrusions 1075A, 1075B are configured to be used with features of tip guide tubes described herein to provide tactile feedback of the location of the tip guide tube and/or to engage the valve seat 1070 with the tip guide tube, as explained below. The features of other valve seats described herein may be included in the valve seats 1070A, 1070B, where applicable. Similarly, the protrusions 1075A, 1075B described herein with respect to the valve seats 1070A, 1070B may be included in the other valve seats described herein.

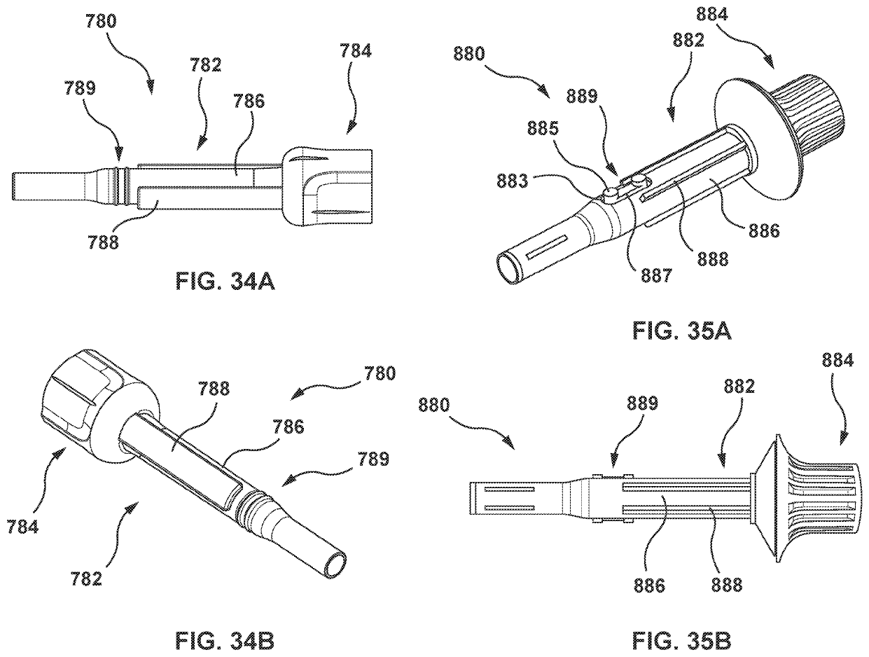

FIGS. 34A and 34B show a tip guide tube 780 according to embodiments herein. The tip guide tube 780 is similar to the tip guide tube 580. Therefore, all of the details of the tip guide tube 780 will not be repeated. Further, the tip guide tube 780 may include any of the features described herein with respect to other embodiments of a tip guide tube. The tip guide tube 780 generally includes a tube 782 and a grip 784. Further, in the embodiment of FIGS. 34A and 34B, the tube 782 includes recesses 786 and ribs 788 extending longitudinally along the tube 782 and disposed alternatingly around an outer surface of the tube 782 along a portion of the length of the tube 782. In the embodiment shown in FIGS. 34A and 34B, there are two recesses 786 and two ribs 788, corresponding to a valve seat that includes two wall portions/legs and two slots. However, this is not meant to be limiting, and the tube 782 may include any number of recesses 786 and ribs 788, preferably corresponding to the number of wall sections/legs and slots in the corresponding valve seat used therewith. In the embodiment of FIGS. 34A and 34B, the tip guide tube 780 further includes a plurality of circumferential ribs or bumps 789 adjacent a proximal end of the longitudinal ribs 786. In the embodiment shown, there are two circumferential ribs 789 and each extends around an entire circumference of the tube 782. However, this is not meant to be limiting, and there may be more or fewer circumferential ribs 789, and the circumferential ribs 789 may only extend around a portion of the circumference of the tube 782. The circumferential ribs 789 interact with the valve seat, such as the valve seat 570, and in particular the wall sections/legs 574 of the valve seat 570. The circumferential ribs 789 may interact with protrusions on the inner surface of the wall sections/legs, such as the protrusions 1075A, 1075B of the valve seats 1070A, 1070B shown in FIGS. 33A-33B. However, this is not required, and the circumferential ribs 789 may simply interact with the free end of the wall sections/legs of the valve seat and/or the inner surface of the wall sections/legs of the valve seat. The circumferential ribs 789 provide tactile feedback to the user of the loading system regarding the location of the tip guide tube 780 relative to the valve seat 570 coupled to the loading ring 550. In particular, the tip guide tube 780 should be retracted a certain distance prior to the capsule 306 being advanced over the transcatheter heart valve prosthesis 200 disposed within the valve seat 570. The tactile feedback provided by the circumferential ribs 789 interacting with the valve seat 570 enables a user to confirm that the tip guide tube 780 is properly retracted prior to advancing the capsule 306 of the delivery system 300. Those skilled in the art will recognize that reference to particular embodiments of the loading system 500 and the components thereof is not meant to be limiting, and that other embodiments of the particular components may also be used with the tip guide tube 780.

FIGS. 35A and 35B show a tip guide tube 880 according to embodiments herein. The tip guide tube 880 is similar to the tip guide tube 580. Therefore, all of the details of the tip guide tube 880 will not be repeated. Further, the tip guide tube 880 may include any of the features described herein with respect to other embodiments of a tip guide tube. The tip guide tube 880 generally includes a tube 882 and a grip 884. Further, in the embodiment of FIGS. 35A and 35B, the tube 882 includes recesses 886 and ribs 888 extending longitudinally along the tube 882 and disposed alternatingly around an outer surface of the tube 882 along a portion of the length of the tube 882. In the embodiment shown in FIGS. 35A and 35B, there are four recesses 886 and four ribs 888, corresponding to a valve seat that includes four wall portions/legs and two slots. However, this is not meant to be limiting, and the tube 882 may include any number of recesses 886 and ribs 888, preferably corresponding to the number of wall sections/legs and slots in the corresponding valve seat used therewith. In the embodiment of FIGS. 35A and 35B, the tip guide tube 880 further includes detents 889 adjacent a proximal end of the longitudinal ribs 886. In the embodiment shown, there are two detents 889 disposed approximately 180 degrees apart from each other around the circumference of the tube 882 (i.e., disposed opposite each other). In the embodiment shown, the detents 889 are disposed in the recesses 886 of the tube 882, between adjacent ribs 888. In the embodiment shown, the detents 889 a located in a cut-out section 883 of the wall of the tube 882. The detents 889 are coupled to the tube via lateral connectors 887. In the embodiment shown, the lateral connectors 887 are at approximately a mid-portion of the length of the detents 889 such that the detents 889 are similar to a “see-saw” in that the longitudinal ends are not attached to the wall of the tube 882. As such, the detents 889 can pivot inwardly and outwardly via the around the lateral connectors 887. Each detent 889 further includes two protrusions 885 extending radially outwardly, with the protrusions 885 of each detent 889 disposed at opposite longitudinal ends of the detent 889. The detents 889, and particularly the protrusions 885 thereof, interact with the valve seat, such as the valve seat 570, and in particular the wall sections/legs 574 of the valve seat 570. The detents 889 may interact with protrusions on the inner surface of the wall sections/legs, such as the protrusions 1075A, 1075B of the valve seats 1070A, 1070B shown in FIGS. 33A-33B. However, this is not required, and the detents 889 may simply interact with the free end of the wall sections/legs of the valve seat and/or the inner surface of the wall sections/legs of the valve seat. The detents 889 provide tactile feedback to the user of the loading system regarding the location of the tip guide tube 880 relative to the valve seat 570 coupled to the loading ring 550. In particular, the tip guide tube 880 should be retracted a certain distance prior to the capsule 306 being advanced over the transcatheter heart valve prosthesis 200 disposed within the valve seat 570. The tactile feedback provided by the detents 889 interacting with the valve seat 570 enables a user to confirm that the tip guide tube 880 is properly retracted prior to advancing the capsule 306 over the transcatheter heart valve prosthesis 200. Those skilled in the art will recognize that reference to particular embodiments of the loading system 500 and the components thereof is not meant to be limiting, and that other embodiments of the particular components may also be used with the tip guide tube 880.

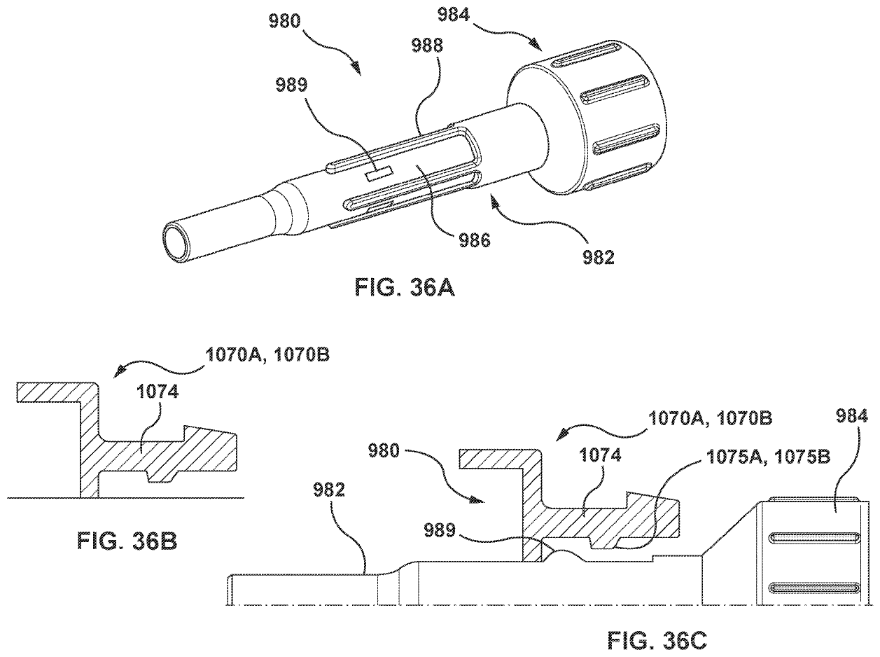

FIGS. 36A-36C show a tip guide tube 980 according to embodiments herein. The tip guide tube 980 is similar to the tip guide tube 580. Therefore, all of the details of the tip guide tube 980 will not be repeated. Further, the tip guide tube 980 may include any of the features described herein with respect to other embodiments of a tip guide tube. The tip guide tube 880 generally includes a tube 982 and a grip 984. Further, in the embodiment of FIGS. 36A-36C, the tube 982 includes recesses 986 and ribs 988 extending longitudinally along the tube 982 and disposed alternatingly around an outer surface of the tube 982 along a portion of the length of the tube 982. In the embodiment shown in FIGS. 36A-36C, there are four recesses 986 and four ribs 988, corresponding to a valve seat that includes four wall portions/legs and four slots. However, this is not meant to be limiting, and the tube 982 may include any number of recesses 986 and ribs 988, preferably corresponding to the number of wall sections/legs and slots in the corresponding valve seat used therewith. In the embodiment of FIGS. 36A-36C, the tip guide tube 980 further includes protrusions 989 extending radially outwardly from the tube 982. In the embodiment shown, the protrusions 989 are disposed in the recesses 986 of the tip guide tube 980. Further, in the embodiment shown, there is a protrusion 989 in each recess 986, but this not meant to be limiting, and only some of the recesses 986 may include protrusions 989. The protrusions 989 are located along a length of the tube 982 such that the protrusions 989 interact with a valve seat, such as the valve seat 570, and in particular the wall sections/legs 574 of the valve seat 570, when the tip guide tube 980 is sufficiently retracted from the valve ring 550 such that the tube 982 does not interfere with radial compression of the transcatheter heart valve prosthesis 200. As shown in FIGS. 36B and 36C, the protrusions 989 may interact with protrusions on the inner surface of the wall sections/legs, such as the protrusions 1075A, 1075B of the valve seats 1070A, 1070B shown in FIGS. 33A-33B. However, this is not required, and the protrusions 989 may simply interact with the free end of the wall sections/legs of the valve seat and/or the inner surface of the wall sections/legs of the valve seat. The protrusions 989 provide tactile feedback to the user of the loading system regarding the location of the tip guide tube 980 relative to the valve seat 570 coupled to the loading ring 550. In particular, the tip guide tube 980 should be retracted a certain distance prior to the capsule 306 being advanced over the transcatheter heart valve prosthesis 200 disposed within the valve seat 570. The tactile feedback provided by the detents 989 interacting with the valve seat 570 enables a user to confirm that the tip guide tube 980 is properly retracted prior to advancing the capsule 306 over the transcatheter heart valve prosthesis 200. Those skilled in the art will recognize that reference to particular embodiments of the loading system 500 and the components thereof is not meant to be limiting, and that other embodiments of the particular components may also be used with the tip guide tube 980.

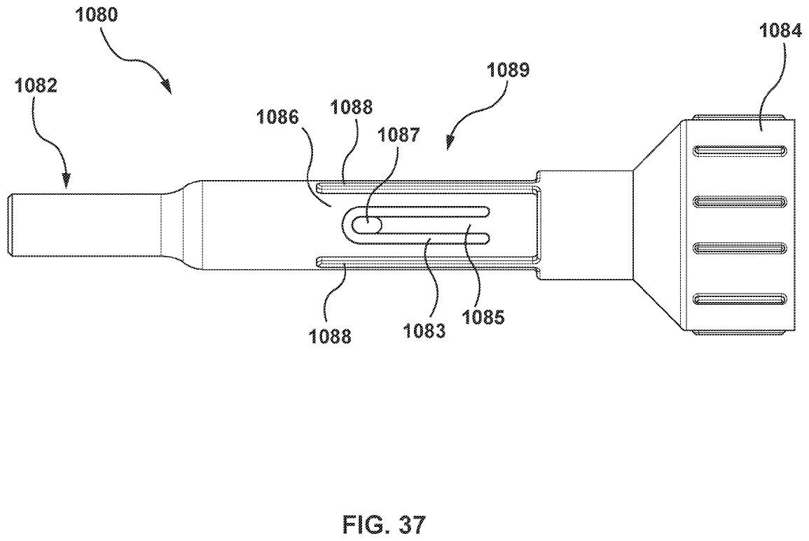

FIG. 37 shows a tip guide tube 1080 according to embodiments herein. The tip guide tube 1080 is similar to the tip guide tube 580. Therefore, all of the details of the tip guide tube 1080 will not be repeated. Further, the tip guide tube 1080 may include any of the features described herein with respect to other embodiments of a tip guide tube. The tip guide tube 1080 generally includes a tube 1082 and a grip 1084. Further, in the embodiment of FIG. 37, the tube 1082 includes recesses 1086 and ribs 1088 extending longitudinally along the tube 1082 and disposed alternatingly around an outer surface of the tube 1082 along a portion of the length of the tube 1082. In the embodiment shown in FIG. 37, there are two recesses 1086 (only one is shown in the side view) and two ribs 1088, corresponding to a valve seat that includes four wall portions/legs and two slots. However, this is not meant to be limiting, and the tube 1082 may include any number of recesses 1088 and ribs 1086, preferably corresponding to the number of wall sections/legs and slots in the corresponding valve seat used therewith. In the embodiment of FIG. 37, the tip guide tube 1080 further includes protrusions 1089 disposed in the recesses 1086 of the tube 1082 (only one protrusion 1089 is shown in FIG. 37 as it is a side view, but a second protrusion 1089 may be disposed opposite the protrusion 1089 shown). The protrusions 1089 are disposed at a longitudinal location along the length of the tube 1082 such that the protrusions 1089 interact with a valve seat, such as the valve seat 570, and in particular the wall sections/legs 574 of the valve seat 570, when the tip guide tube 1080 is sufficiently retracted from the valve ring 550 such that the tube 1082 does not interfere with radial compression of the transcatheter heart valve prosthesis 200. In the embodiment shown in FIG. 37, each protrusion 1089 is formed of a cantilever 1085 coupled to the tube 1082 at a first end of the cantilever 1085, with the cantilever 1085 having a free second end opposite the first end. The cantilever 1085 is disposed at a cut-out 1083 of the wall of the tube 1082 such that the free second end can move radially inwardly and outwardly. The free second end of the cantilever 1085 includes a radially outward protrusion 1087. In the embodiment shown, the protrusions 1089, and particularly the radially outward protrusions 1087 thereof, interact with the valve seat, such as the valve seat 570, and in particular the wall sections/legs 574 of the valve seat 570. The protrusions 1089 may interact with protrusions on the inner surface of the wall sections/legs, such as the protrusions 1075A, 1075B of the valve seats 1070A, 1070B shown in FIGS. 33A-33B. However, this is not required, and the protrusions 1089 may simply interact with the free end of the wall sections/legs of the valve seat and/or the inner surface of the wall sections/legs of the valve seat. The protrusions 1089 provide tactile feedback to the user of the loading system regarding the location of the tip guide tube 1080 relative to the valve seat 570 coupled to the loading ring 550. In particular, the tip guide tube 1080 should be retracted a certain distance prior to the capsule 306 being advanced over the transcatheter heart valve prosthesis 200 disposed within the valve seat 570. The tactile feedback provided by the protrusions 1089 interacting with the valve seat 570 enables a user to confirm that the tip guide tube 1080 is properly retracted prior to advancing the capsule 306 over the transcatheter heart valve prosthesis 200. Those skilled in the art will recognize that reference to particular embodiments of the loading system 500 and the components thereof is not meant to be limiting, and that other embodiments of the particular components may also be used with the tip guide tube 880.

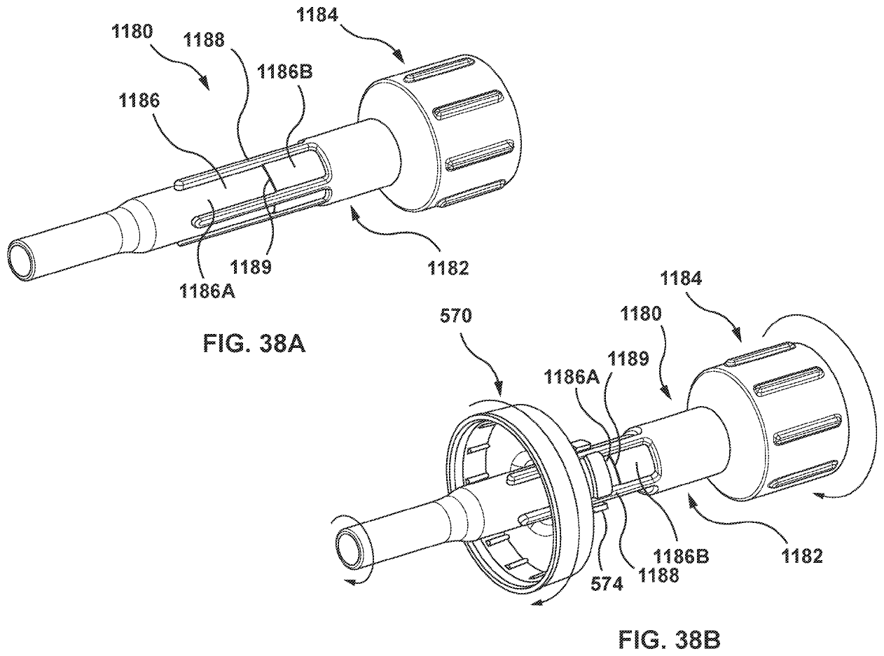

FIGS. 38A-38C show a tip guide tube 1180 according to embodiments herein. The tip guide tube 1180 is similar to the tip guide tube 580. Therefore, all of the details of the tip guide tube 1180 will not be repeated. Further, the tip guide tube 1180 may include any of the features described herein with respect to other embodiments of a tip guide tube. The tip guide tube 1180 generally includes a tube 1182 and a grip 1184. Further, in the embodiment of FIGS. 38A-38C, the tube 1182 includes recesses 1186 and ribs 1188 extending longitudinally along the tube 1182 and disposed alternatingly around an outer surface of the tube 1182 along a portion of the length of the tube 1182. In the embodiment shown in FIGS. 38A-38C, there are four recesses 1186 and four ribs 1188, corresponding to a valve seat that includes four wall portions/legs and two slots. However, this is not meant to be limiting, and the tube 1182 may include any number of recesses 1186 and ribs 1188, preferably corresponding to the number of wall sections/legs and slots in the corresponding valve seat used therewith. In the embodiment of FIGS. 38A-38C, the tip guide tube 1180 further includes a ledge or shoulder 1189 extending circumferentially around the circumference of the tube 1182 in the recesses 1186. In particular, in the embodiment shown, the tube 1182 at the recesses 1186 in a direction away from the grip 1184 (i.e., at 1186A) has a first outer diameter. The shoulder 1189 is a step such that in a direction towards the grip 1184 from the shoulder 1189 (i.e., at 1186B), the tube 1182 at the recesses 1186 has a second outer diameter larger than the first outer diameter. In the embodiment shown, the shoulder 1189 interacts with the valve seat, such as the valve seat 570, and in particular the wall sections/legs 574 of the valve seat 570. The shoulder 1089 provides tactile feedback to the user of the loading system regarding the location of the tip guide tube 1180 relative to the valve seat 570 coupled to the loading ring 550. In particular, the tip guide tube 1180 should be retracted a certain distance prior to the capsule 306 being advanced over the transcatheter heart valve prosthesis 200 disposed within the valve seat 570. The tactile feedback provided by the shoulder 1189 interacting with the valve seat 570 enables a user to confirm that the tip guide tube 1180 is properly retracted prior to advancing the capsule 306 over the transcatheter heart valve prosthesis 200. Thus, the location of the shoulder 1189 along the length of the tube 1182 is such that when the wall portions/legs of the valve seat move from the second outer diameter to the first outer diameter, tactile feedback is provided and the tip guide tube 1182 is properly retracted. Those skilled in the art will recognize that reference to particular embodiments of the loading system 500 and the components thereof is not meant to be limiting, and that other embodiments of the particular components may also be used with the tip guide tube 1180.

FIGS. 39A and 39B show a tip guide tube 1280 according to embodiments herein. The tip guide tube 1280 is similar to the tip guide tube 580. Therefore, all of the details of the tip guide tube 1280 will not be repeated. Further, the tip guide tube 1280 may include any of the features described herein with respect to other embodiments of a tip guide tube. The tip guide tube 1280 generally includes a tube 1282 and a grip 1284. Further, in the embodiment of FIGS. 39A-39B, the tube 1282 includes recesses 1286 and ribs 1288 extending longitudinally along the tube 1282 and disposed alternatingly around an outer surface of the tube 1282 along a portion of the length of the tube 1282. In the embodiment shown in FIGS. 39A-39B, there are four recesses 1286 and four ribs 1288, corresponding to a valve seat that includes four wall portions/legs and four slots. However, this is not meant to be limiting, and the tube 1282 may include any number of recesses 1286 and ribs 1288, preferably corresponding to the number of wall sections/legs and slots in the corresponding valve seat used therewith. In the embodiment of FIGS. 39A-39B, two of the ribs 1288 extend further from the grip 1284 than the other two ribs 1288. Further, the longer ribs 1288 include protrusions 1289 extending radially outwardly from the corresponding rib 1288. The protrusions 1289 are located lengthwise along the corresponding rib 1288 to provide tactile feedback to the user such that the user knows that the tip guide tube 1280 is sufficiently retracted from the valve ring 550 such that the tube 1282 does not interfere with radial compression of the transcatheter heart valve prosthesis 200. In the embodiment shown, two of the ribs 1288 opposite each other around the circumference of the tube 1282 are lengthened and include the radial protrusions 1289. However, this is not meant to be limiting, and in other embodiments, more or fewer of the ribs 1288 may be lengthened and/or may include radial protrusions 1289. In another example, all of the ribs 1288 may be of the same length, and some or all of the ribs 1288 may include the radial protrusions 1289. The radial protrusions 1289 may interact with the valve seat to provide tactile feedback. For example, and not by way of limitation, the radial protrusions 1289 may interact with the base 571 of the valve seat 570 or the extensions 778 of the valve seat 770. However, this is not meant to be limiting, and the radial protrusions 1289 may interact with any of the valve seats disclose herein, and variations thereof, or other components of the loading system. As shown in FIGS. 39A-39B, the rib 1288 may include a series of protrusions 1289 indicating a “safe zone” for the retraction of the tip guide tube 1280. Those skilled in the art will recognize that reference to particular embodiments of the loading system 500 and the components thereof is not meant to be limiting, and that other embodiments of the particular components may also be used with the tip guide tube 1280.

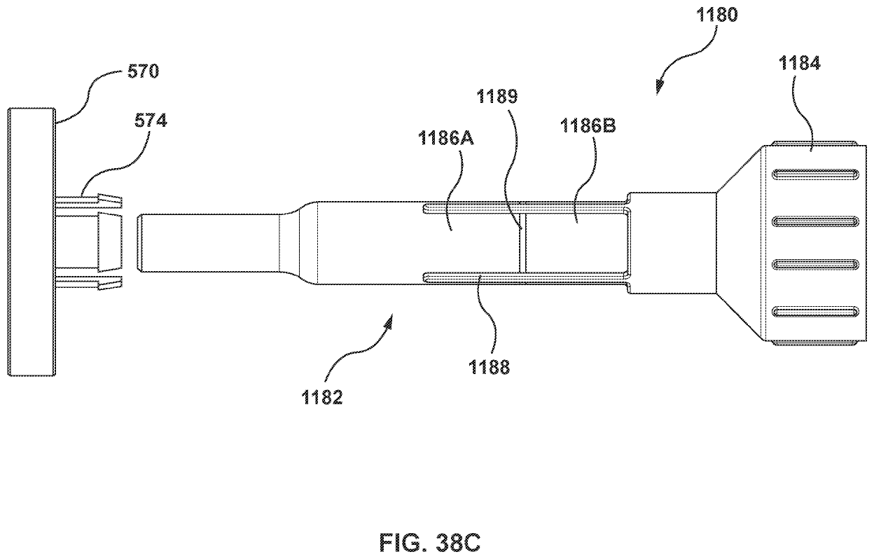

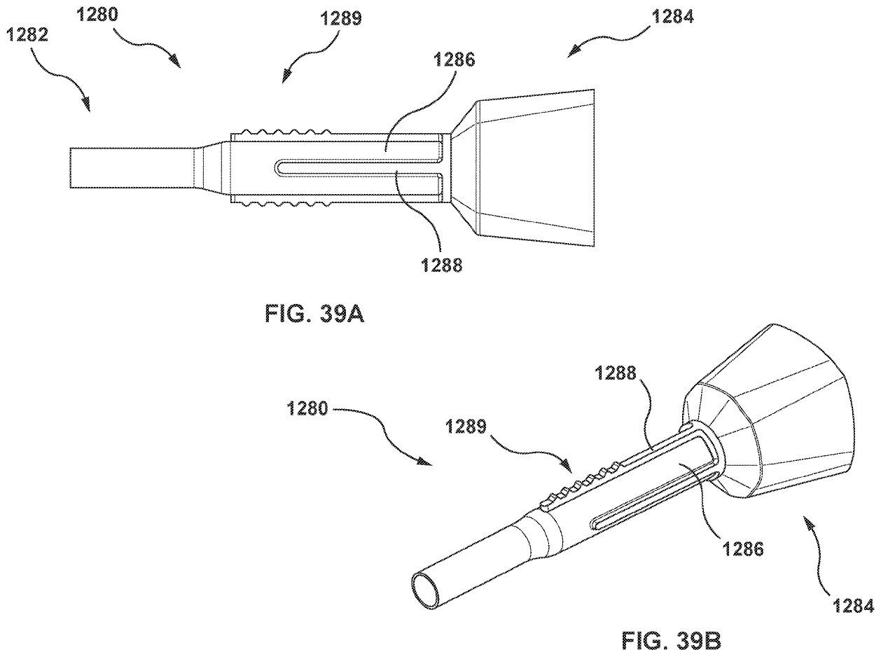

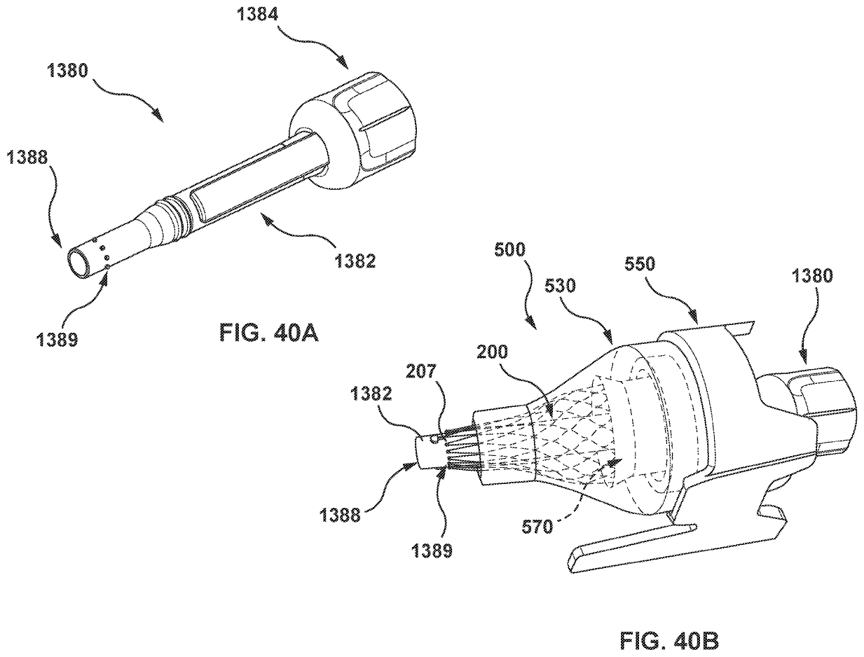

FIGS. 40A and 40B show a tip guide tube 1380 according to embodiments herein. The tip guide tube 1380 is similar to the tip guide tube 580. Therefore, all of the details of the tip guide tube 1380 will not be repeated. Further, the tip guide tube 1380 may include any of the features described herein with respect to other embodiments of a tip guide tube. The tip guide tube 1380 generally includes a tube 1382 and a grip 1384. Further, in the embodiment of FIGS. 40A-40B, the tube 1382 includes bumps or pins 1389 extending radially outwardly for the tube 1382 adjacent an end 1388 thereof. In particular, in the embodiment shown, the pins 1389 are disposed equidistantly around the circumference of the tube 1382. As can be seen in FIG. 40B, the end 1388 of the tip guide tube 1380 is the end that extends through the loading ring 550, the valve seat 570, the loading cone 530, and the transcatheter heart valve prosthesis 200 and protrudes past smaller diameter end of the loading cone 530. As explained above with respect to FIGS. 8K and 8L, the end 1388 of the tip guide tube 1380 prevents the outflow end 210 of the transcatheter heart valve prosthesis 200 from collapsing radially inward, i.e., maintains the outflow end 210 of the transcatheter heart valve prosthesis 200 at an inner diameter substantially equal to the outer diameter of the tube 1382 of the tip guide tube 1380 at the end 1388. This eases coupling of the tabs 220 to the tab pockets 318 of the delivery system 300. The pins 1389 enable a user to align the crowns 207 at the outflow end 210 of the transcatheter heart valve prosthesis 200 with the pins 1389. In particular, the user can manually manipulate the crowns 207 around the pins 1389. Wrapping the crowns 207 around the pins 1389 ensures equidistant spacing of the crowns 207 of the outflow end 210 of the transcatheter heart valve prosthesis 200, thereby mitigating potential outflow crown crossing on the crimped transcatheter heart valve prosthesis 200. Other features of the tip guide tube 1380 are not described herein, and may be as described herein for other embodiments.

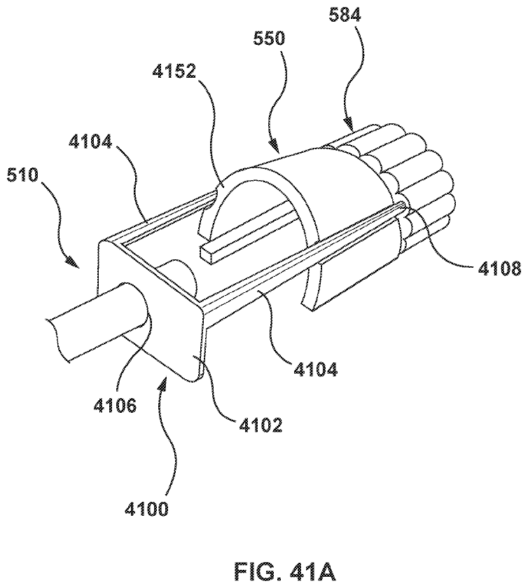

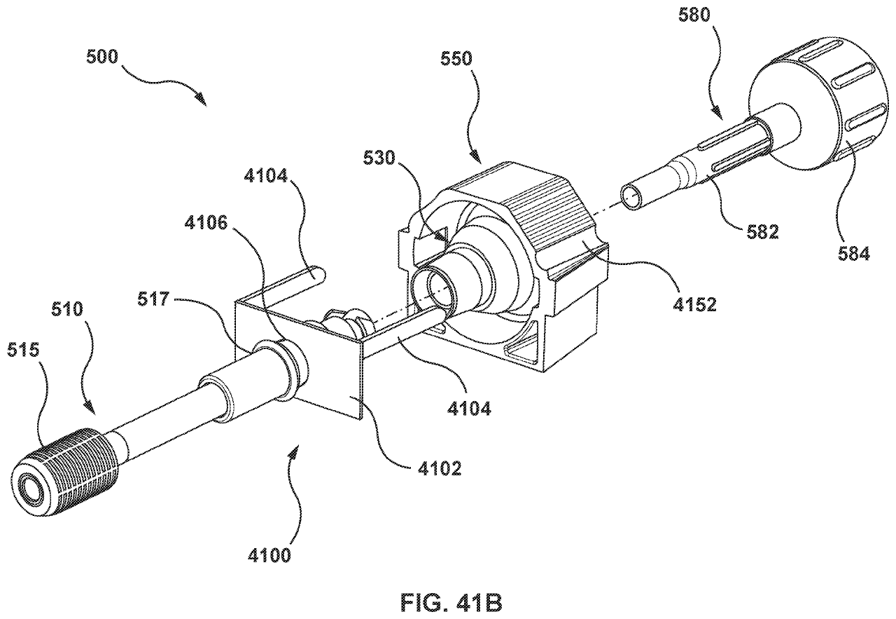

FIGS. 41A-41B show a pusher 4100 according to embodiments herein. Those skilled in the art would understand that the pusher 4100 may include additional components not described herein, or the components may be modified or removed in keeping with description herein. The pusher 4100 includes a plate 4102 and a plurality of arms 4104 extending distally from the plate 4102. In the embodiment shown, there are two arms 4104, but this is not meant to be limiting, and more or fewer arms 4104 may be utilized in keeping with the purpose of the arms 4104 described herein. The plate 4102 includes an opening 4106 extending therethrough. As shown in FIGS. 41A-41B, the opening 4106 is configured to receive the capsule guide tube 510 of the loading system 500. Further, the arms 4104 are configured to extend around and past the loading ring 550 of the loading system 500. For example, and not by way of limitation, the loading ring 550 may include grooves 4152 extending longitudinally and configured to receive the arms 4104 therein. As shown in FIG. 41A, the distal ends 4108 of the arms 4104 engage the grip 584 of the tip guide tube 580. Therefore, when the capsule guide tube 510 is advanced with the capsule 306 of the delivery system 300, as described above with respect to the FIGS. 8T-8V, the pusher 4100 advances with the capsule guide tube 510, thereby pushing the grip 584 of the tip guide tube 580. The pusher 4100 thereby ensures that the tip guide tube 580 is retracted such that the tube 582 of the tip guide tube 580 does not interfere with radial compression of the transcatheter heart valve prosthesis 200.

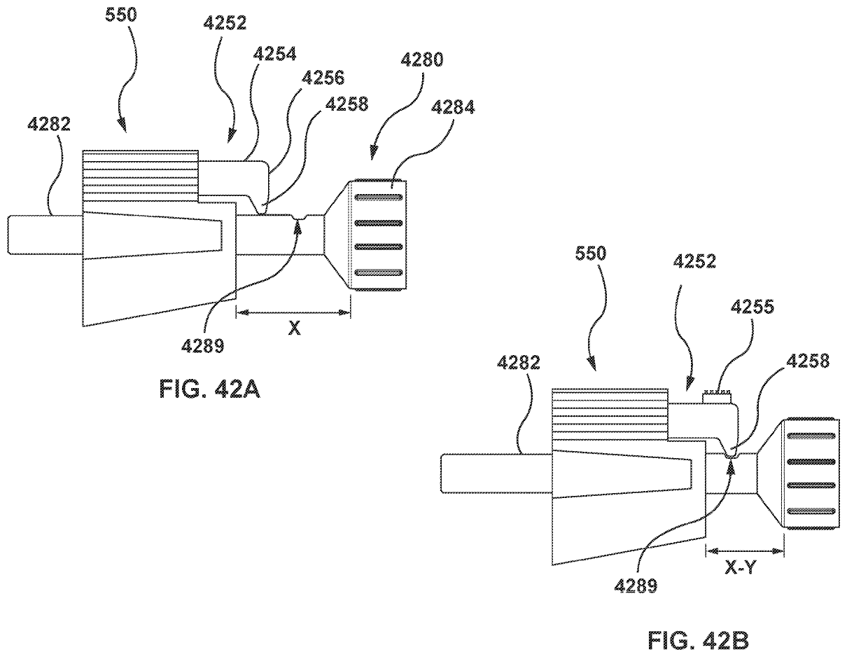

FIGS. 42A-42B show a tip guide tube 4280 according to embodiments herein. The tip guide tube 4280 is similar to the tip guide tube 580. Therefore, all of the details of the tip guide tube 4280 will not be repeated. Further, the tip guide tube 4280 may include any of the features described herein with respect to other embodiments of a tip guide tube. The tip guide tube 4280 generally includes a tube 4282 and a grip 4284. Adjacent the grip 4284 the tube 4282 includes a notch 4289. The notch 4289 is configured to interact with a slider 4252. The slider 4252 may be an extension of the loading ring 550, as shown in FIGS. 42A-42B. The slider 4252 includes a longitudinal portion 4254 and a radial extension portion 4256. The radial extension portion includes a shaped end 4258 configured to fit in the notch 4289 of the tip guide tube 4280. The tip guide tube 4280 includes a first position, shown in FIG. 42A, wherein the notch 4289 is distal of the shaped end 4258 of the slider 4252. In this first portions, the tip guide tube 4280 may be rotated to align the tabs 220 of the transcatheter heart valve prosthesis 200 with the tab pockets 318 of the spindle 310 of the delivery system 300, as described above. With the tabs 220 aligned with the tab pockets 318, the tip guide tube 4280 may be advanced proximally (i.e., to the left in FIG. 42A) such that the shaped end 4258 of the slider 4258 is disposed in the notch 4289, as shown in FIG. 42B. With the shaped end 4258 disposed in the notch 4289, the tip guide tube 4250 may only be retracted (i.e., to the right in FIG. 42B). The slide 4252 may include a button 4255 for the user to engage to slide the tip guide tube distally (i.e., to the right in FIG. 42B) to ensure that the tube 4282 of the tip guide tube 4280 does not interfere with radial compression of the transcatheter heart valve prosthesis 200.

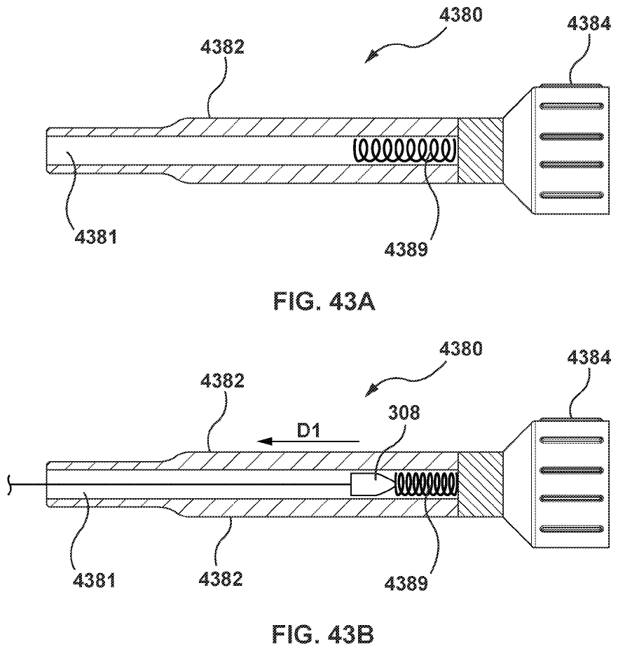

FIGS. 43A-43B show a tip guide tube 4380 according to embodiments herein. The tip guide tube 4380 is similar to the tip guide tube 580. Therefore, all of the details of the tip guide tube 4380 will not be repeated. Further, the tip guide tube 4380 may include any of the features described herein with respect to other embodiments of a tip guide tube. The tip guide tube 4380 generally includes a grip 4384 and a tube 4382 extending proximally from the grip 4384. The tube 4282 includes a lumen 4281 extending from the proximal end thereof. As explained above with respect to FIGS. 8M-8N, when the delivery device 300 is inserted through the loading system (100, 500), the tip 308 of the delivery system 300 extends within the lumen 4381 of the tip guide tube 4280. In the embodiment of FIGS. 43A-43B a spring 4389 is disposed in the lumen 4381. Thus, when the tip 308 of the delivery system 300 is advanced into the lumen 4381, the tip 4380 compresses the spring 4389. Thus, a force must be exerted on the tip guide tube 4380 in a first direction D1 to maintain the tip guide tube 4380 in the proper position to aid in preventing collapse of the crowns 207 at the outflow end 210 of the transcatheter heart valve prosthesis 200 and to assist in locating the tabs 220 in the tab pockets 318, as explained above. When the tabs 220 are located in the tab pockets 318, the tip guide tube 4380 may be released. The force of the spring 4389 returning to its expanded state as shown in FIG. 43A will automatically force the tip guide tube 4380 distally, thereby retracting the tube 4382 of the tip guide tube 4380 sufficiently such that it does not interfere with radial compression of the transcatheter heart valve prosthesis 200.

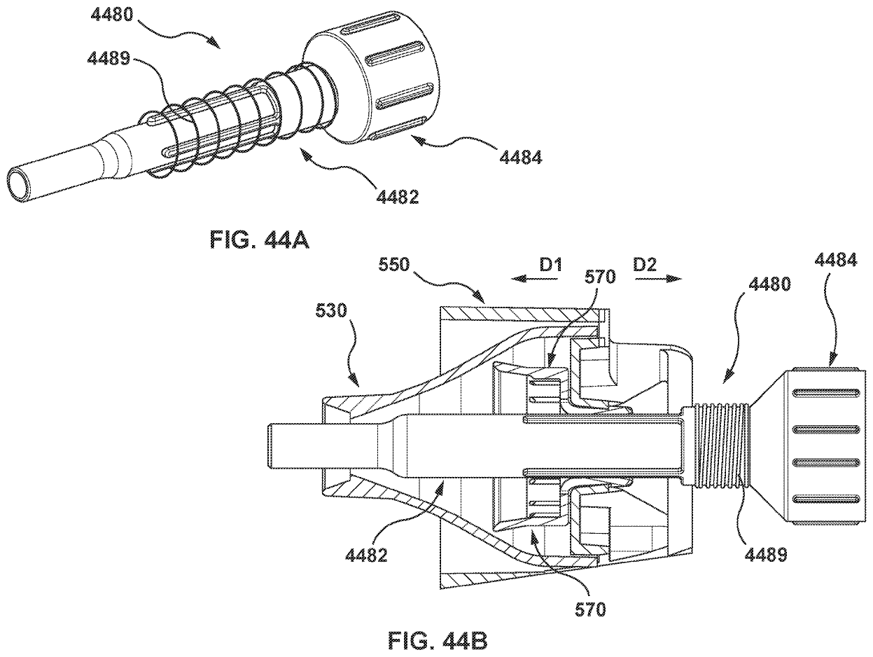

FIGS. 44A-44B show a tip guide tube 4480 according to embodiments herein. The tip guide tube 4480 is similar to the tip guide tube 580. Therefore, all of the details of the tip guide tube 4480 will not be repeated. Further, the tip guide tube 4480 may include any of the features described herein with respect to other embodiments of a tip guide tube. The tip guide tube 4480 generally includes a tube 4482 and a grip 4484. Further, in the embodiment of FIGS. 44A-44B, the tip guide tube 4480 includes a spring 4489 disposed around an exterior of the tube 4482. In the embodiment shown, a first end of the spring 4489 as coupled to the tube 4482 or the grip 4484 adjacent the grip 4484 and a second end of the spring 4489 extends towards a proximal end of the tube 4482, as shown in FIG. 44A. When the tip guide tube 4482 is inserted through the loading ring 550, the valve seat 570, and the loading cone 530 of the loading system 500, as shown in FIG. 44B, the spring 4489 compresses against the loading ring 550 and the grip 4484. Thus, a force must be exerted on the tip guide tube 4480 in a first direction D1 to maintain the tip guide tube 4480 in the position shown in FIG. 44B to aid in preventing collapse of the crowns 207 at the outflow end 210 of the transcatheter heart valve prosthesis 200 and to assist in locating the tabs 220 in the tab pockets 318, as explained above. When the tabs 220 are located in the tab pockets 318, the tip guide tube 4480 may be released. The force of the spring 4489 returning to its expanded state as shown in FIG. 43A will automatically force the tip guide tube 4480 distally (i.e. in a second direction D2 opposite the first direction D1), thereby retracting the tube 4482 of the tip guide tube 4480 sufficiently such that it does not interfere with radial compression of the transcatheter heart valve prosthesis 200.

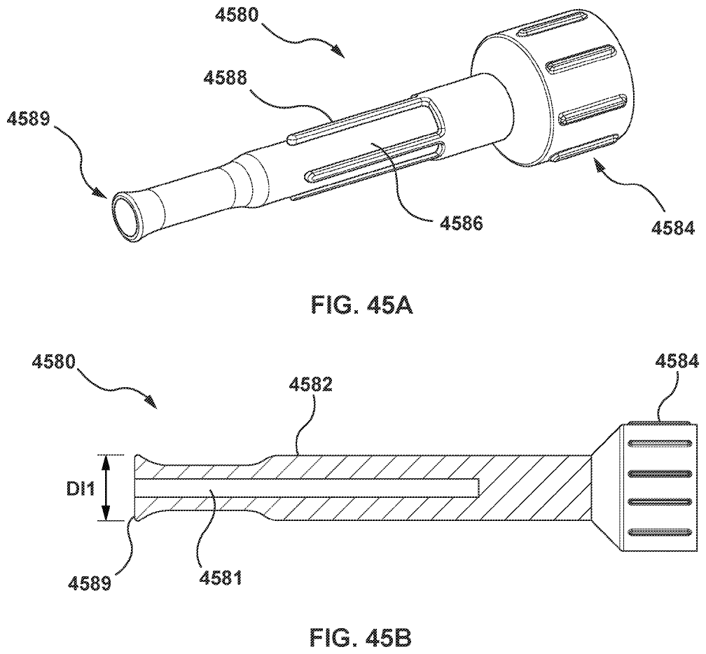

FIGS. 45A-45B show a tip guide tube 4580 according to embodiments herein. The tip guide tube 4580 is similar to the tip guide tube 580. Therefore, all of the details of the tip guide tube 4580 will not be repeated. Further, the tip guide tube 4580 may include any of the features described herein with respect to other embodiments of a tip guide tube, such as recesses 4586 and ribs 4588. The tip guide tube 4580 generally includes a tube 4582 and a grip 4584. In the embodiment of FIGS. 44A-44B, the tube 4582 includes an enlarged or flared end 4589 opposite the grip 4484 (i.e., the proximal end or the end inserted into the loading ring 550). The flared end 4589 has a diameter DI1 that is larger than a diameter of the distal end of the capsule 308 of the delivery system 300. The flared end 4589 prevents the tube 4582 of the tip guide tube 4580 from entering into the distal end of the capsule 308 of the delivery system 300. Additionally, a lumen 4581 of the tube 4582 may be tapered in the distal direction (i.e., the diameter of the lumen 4581 tapers in a direction away from the flared end 4589).

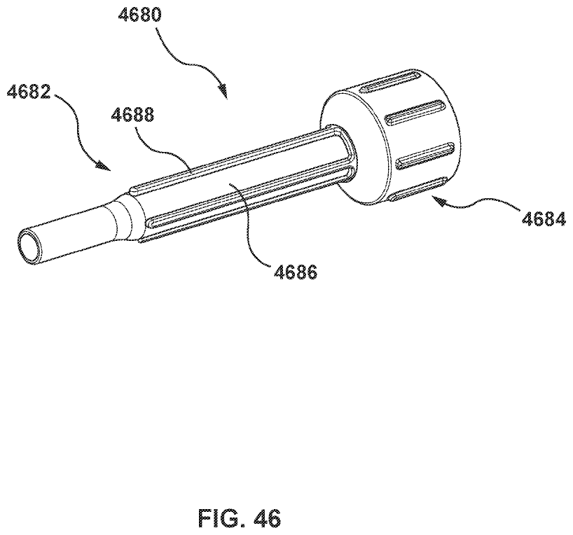

FIG. 46 shows a tip guide tube 4680 according to embodiments herein. The tip guide tube 4680 is similar to the tip guide tube 580. Therefore, all of the details of the tip guide tube 4680 will not be repeated. Further, the tip guide tube 4680 may include any of the features described herein with respect to other embodiments of a tip guide tube. The tip guide tube 4680 generally includes a tube 4682 and a grip 4684. Further, in the embodiment of FIG. 46, the tube 4682 includes recesses 4686 and ribs 4688 extending longitudinally along the tube 4682 and disposed alternatingly around an outer surface of the tube 4682 along a portion of the length of the tube 4682. In the embodiment of FIG. 46, as compared to the tip guide tube 580 shown in FIGS. 22 and 23, the recesses 4686 and ribs 4688 extend along the tube 4682 to the grip 4684, whereas the recesses 681 and ribs 682 of the tip guide tube 580 are spaced from the grip 584. Further, the ribs 4688 of FIG. 46 extend further proximally (opposite the grip 4684) than the ribs 682 of the tip guide tube 580. Thus, the overall length of the tip guide tube 4680 may be shorter than the tip guide tube 580, while maintaining approximately the same length of the ribs 4688, 682. A reduced overall length of the tip guide tube 4680 reduces the risk of the tip guide tube 4680 extending to the capsule 306 of the delivery system 300 during loading.

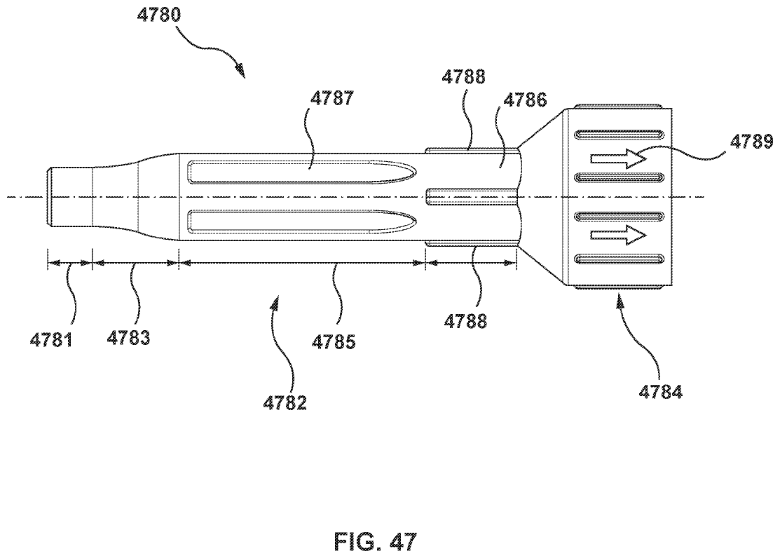

FIG. 47 shows a tip guide tube 4780 according to embodiments herein. The tip guide tube 4780 is similar to the tip guide tube 580. Therefore, all of the details of the tip guide tube 4780 will not be repeated. Further, the tip guide tube 4780 may include any of the features described herein with respect to other embodiments of a tip guide tube. The tip guide tube 4780 generally includes a tube 4782 and a grip 4784. Further, in the embodiment of FIG. 47, the tube 4782 includes recesses 4786 and ribs 4788 extending longitudinally along the tube 4782 and disposed alternatingly around an outer surface of the tube 4782 along a portion of the length of the tube 4782. In the embodiment of FIG. 46, as compared to the tip guide tube 580 shown in FIGS. 22 and 23, the ribs 4788 extend along the tube 4782 to the grip 4784, and extend away from the grip 4784 a shorter distance, as shown at in FIG. 47. The tube 4782 includes a paddle section 4781 at an end thereof opposite the grip 4784. The paddle section 4781 is generally cylindrical and is configured to accommodate the crowns 207 and the paddles 220 at the outflow end of the transcatheter heart valve prosthesis 200, as described above. The tube 4782 includes an outwardly flared section 4783 distal to the paddle section 4781. The tube 4782 further includes a generally cylindrical section 4785 disposed between the flared section 4783 and the grip 4784. The section 4785 includes a plurality of cutouts 4787 through the wall of the tube 4782. The cutouts 4787 significantly reduce the overall weight of the tip guide tube 4780 such that the tip guide tube 4780 floats in a saline bath. Accordingly, when the tip guide tub 4780 is used in the method described above with respect to FIGS. 8A-8Y, and particularly with respect to FIGS. 80-8R, the tip guide tube 4780 may float out of the loading cone 530, valve seat 570, and loading ring 530, thereby automatically retracting the tube 4782 of the tip guide tube 4780 sufficiently such that it does not interfere with radial compression of the transcatheter heart valve prosthesis 200. Also shown in the embodiment of FIG. 47 are arrows 4789 disposed on the grip 4784 of the tip guide tube 4780. The arrows 4789 serve as reminders for users to withdraw the tip guide tube 4780 from the remainder of the loading system 500 prior to compressing the transcatheter heart valve prosthesis 200. The arrows 4789 may be used in any of the embodiments described herein, as may the other features described with respect to the tip guide tube 4780.

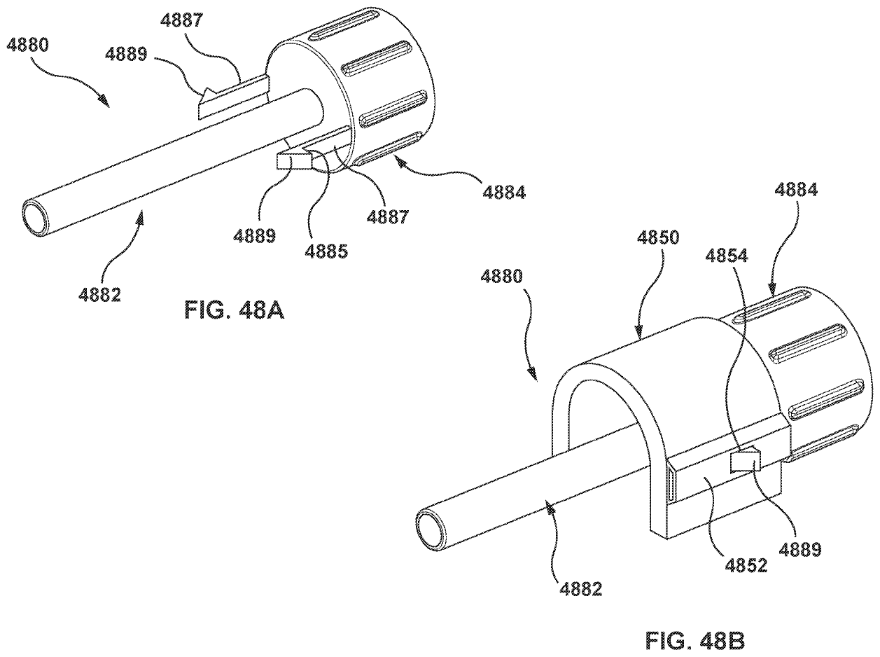

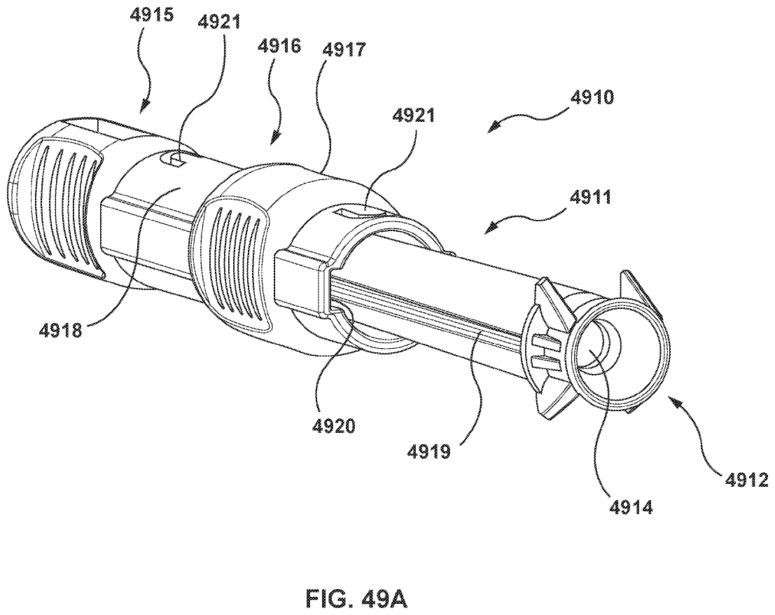

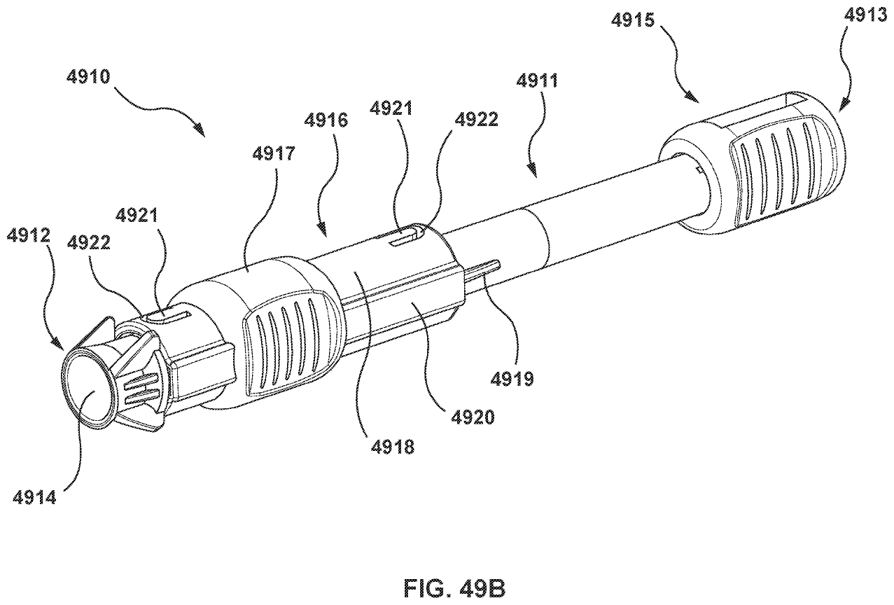

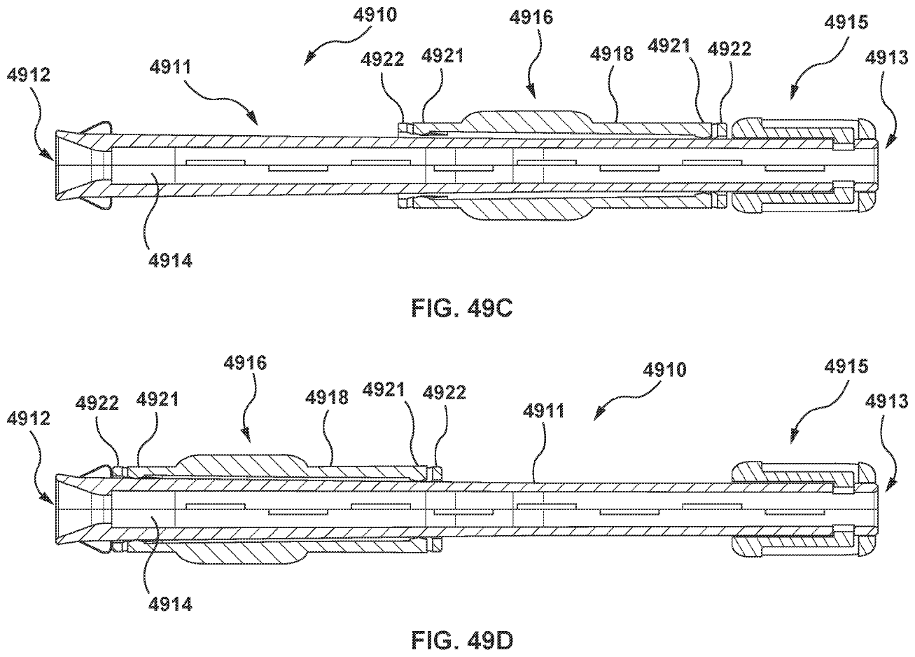

FIGS. 48A-48B show a tip guide tube 4880 according to embodiments herein. The tip guide tube 4880 is similar to the tip guide tube 580. Therefore, all of the details of the tip guide tube 4880 will not be repeated. Further, the tip guide tube 4880 may include any of the features described herein with respect to other embodiments of a tip guide tube. The tip guide tube 4880 generally includes a tube 4882 and a grip 4884. The tube 4882 may include recesses (not shown), ribs (not shown), and a lumen as described in other embodiments herein. In the embodiment shown in FIGS. 48A-48B, the tip guide tube 4880 includes arms 4887 extending proximally from the grip 4884 (i.e., in the same direction as the tube 4882). In the embodiment shown, the tip guide tube 4880 includes two arms 4887 disposed approximately 180 degrees apart around the circumference of the grip 4884. Further, the arms 4887 are each disposed adjacent the outer perimeter of the grip 4884, as shown. Each arm 4887 includes a clip 4889 at a proximal end thereof. Each clip 4889 is an radially outward projection that includes a sloped surface 4885 such that the clip 4889 at the proximal end projects more radially outwardly than adjacent the arm 4887, as shown in FIGS. 48A and 48B. FIG. 48B shows the tip guide tube 4880 disposed within a loading ring 4850, as described above with respect to FIGS. 1A, 8J-8N, and 14. However, the loading ring 4850 includes slots 4852 configured to receive the arms 4887 of the tip guide tube 4880 and openings 4854 configured to receive the clips 4889 of the tip guide tube 4800. In particular, as shown in FIG. 48B, each arm 4887 is inserted into a corresponding slot 4852 at a distal end of the loading ring 4850, with the corresponding clip 4889 entering the slot 4852 first. When the tip guide tube 4880 is advanced such that the clips 4889 align with the corresponding openings 4854 of the loading ring 4850, the clips 4889 pop out of the corresponding openings 4854, as shown in FIG. 48B. When it is time for the tip guide tube 480 to be retracted such that it does not interfere with radial compression of the transcatheter heart valve prosthesis 200, the clips 4889 may be squeezed together, thereby automatically retracting the tip guide tube 4880.

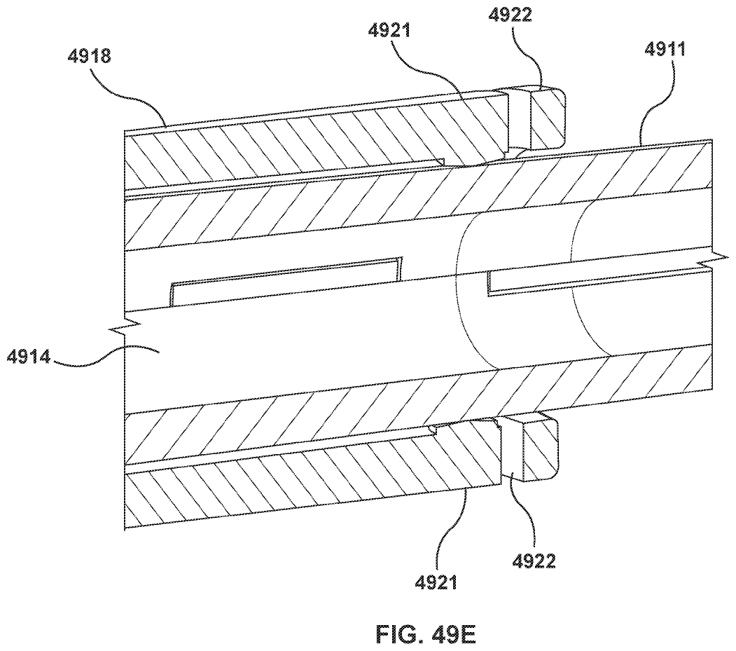

FIGS. 49A-49E show a capsule guide tube 4910 according to embodiments hereof. The capsule guide tube 4910 may be similar to the capsule guide tube 510 described above. Therefore, all of the details of the capsule guide tube 4910 will not be described. The capsule guide tube 4910 generally includes a tube 4911 having a first (distal) end 4912, a second (proximal) end 4913, and a passageway or lumen 4914 extending from the first end 4912 to the second end 4913 such that the capsule guide tube 4910 can be disposed over the capsule 306 of the delivery system 300, as described above. The capsule guide tube 4910 further includes a grip 4915 coupled to an exterior of the tube 4911, and a locking member 4916 configured to lock the capsule guide tube 4910 to the capsule 306 of the delivery system 300, as explained above. The locking member 4916 is disposed around the tube 4911 and includes a lock grip 4917 and a lock tube 4918. One skilled in the art will realize that FIGS. 49A-49D illustrate one example of a capsule guide tube and that existing components illustrated in FIGS. 49A-49E may be removed and/or additional components may be added to the capsule guide tube 4910.

The tube 4911 of the capsule guide tube 4910 further includes guide rails 4919 extending radially outwardly and longitudinally along at least a portion of the tube 4911. In the embodiment of FIGS. 49A-49E, there are two guide rails 4919 disposed diametrically opposed from each other. However, this is not meant to be limiting, and more or fewer guide rails 4919 may be utilized. The locking member 4916 further includes grooves 4920 along an inner surface thereof that are configured to receive the guide rails 4919 therein. Accordingly, in the embodiment shown, there are two grooves 4920 with each one receiving a corresponding one of the guide rails 4919 therein. The guide rails 4919 and grooves 4920 prevent out of axis movement (e.g., rotational movement) of the locking member 4916 when moving from an unlocked configuration shown in FIGS. 49A and 49C to a locked configuration shown in FIGS. 49B and 49D. In some embodiments, the rails 4919 may have an outward taper to minimize rattle of the locking member 4916 when the locking member 4916 is in the locked configuration.

The locking member 4916 of the capsule guide tube 4910 further includes cantilevers 4921 formed by cutouts 4922 in the lock tube 4918 of the locking member 4916. As can be seen in FIGS. 49A and 49B, each cantilever 4921 extends in a longitudinal direction and is formed by the cutouts 4922 being U-shaped. Further, in the embodiment shown, there are four cantilevers 4922, with two on a proximal side of the lock grip 4917 and two on a distal side of the lock grip 4917. Further the corresponding two cantilevers 4921 on each side of the lock grip 4917 are disposed diametrically opposite each other. In other words, a first cantilever 4921 is disposed on the proximal side of the lock grip 4917. A second cantilever 4921 is also disposed on the proximal side of the lock grip 4917 and is disposed diametrically opposite the first cantilever 4921. Similarly, a third cantilever 4921 is disposed on the distal side of the lock grip 4917. A fourth cantilever 4921 is also disposed on the distal side of the lock grip 4917 and is disposed diametrically opposite the third cantilever 4921. However, this is not meant to be limiting, and other arrangements of the cantilevers 4921 are contemplated. The cantilevers 4921 assist in accommodating a dynamic system. In other words, the capsule guide tube 4910 needs to lock into place on the capsule 306 of the delivery system 300 with the capsule 306 in an unloaded state (i.e., without the transcatheter heart valve prosthesis 200 loaded into the capsule 306) and a loaded state (i.e., with the transcatheter heart valve prosthesis 200 loaded into the capsule 306). The diameter of the capsule 306 changes from the unloaded state to the loaded state (e.g., approximately 0.1 mm of expansion). The cantilevers 4921 exert a force on the two halves of the tube 4911 to keep the tube 4911 closed during initial loading of the tabs 220 of the heart valve prosthesis 200. As the remainder of the transcatheter heart valve prosthesis 200 is being loaded into the capsule 306, radial outward force on the capsule guide tube 4910 increases. As the force increases, the cantilevers 4921 are configured to flex to allow the two halves of the tube 4911 to expand (i.e., move away from each other), reducing high loading forces while keeping the tabs 220 of the transcatheter heart valve prosthesis 200 within the tab pockets 318 of the spindle 310.

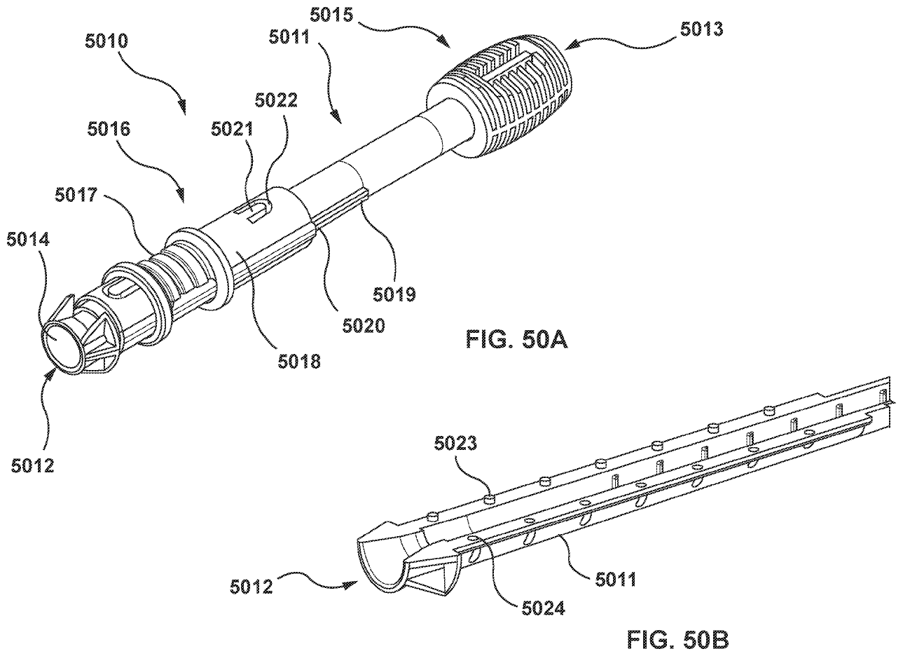

FIGS. 50A-50B show a capsule guide tube 5010 according to embodiments hereof. The capsule guide tube 5010 may be similar to the capsule guide tube 510 and the capsule guide tube 4910 described above. Therefore, all of the details of the capsule guide tube 5010 will not be described. The capsule guide tube 5010 generally includes a tube 5011 having a first (distal) end 5012, a second (proximal) end 5013, and a passageway or lumen 5014 extending from the first end 5012 to the second end 5013 such that the capsule guide tube 5010 can be disposed over the capsule 306 of the delivery system 300, as described above. The capsule guide tube 5010 further includes a grip 5015 coupled to an exterior of the tube 5011, and a locking member 5016 configured to lock the capsule guide tube 5010 to the capsule 306 of the delivery system 300, as explained above. The locking member 5016 is disposed around the tube 5011 and includes a lock grip 5017 and a lock tube 5018. Similar to the embodiment of FIGS. 49A-49E, the capsule guide tube 5010 further includes guide rails 5019 on the tube 5011 and grooves 5020 in the lock tube 5018. The capsule guide tube further includes cantilevers 5021 and grooves 5022 on the lock tube 5018 as described above. One skilled in the art will realize that FIGS. 50A-50B illustrate one example of a capsule guide tube and that existing components illustrated in FIGS. 50A-50B may be removed and/or additional components may be added to the capsule guide tube 5010. As described above with respect to the tube 511, the tube 5011 includes two halves split longitudinally. In the embodiment of FIGS. 50A-50B, each half of the tube 5011 includes pins 5023 and/or recesses 5024. The recesses 5024 are configured to receive the pins 5023 of the other half of the tube 5011 to coupled the halves together, as described above with respect to the tabs 516 and recesses 617 of the tube 511.

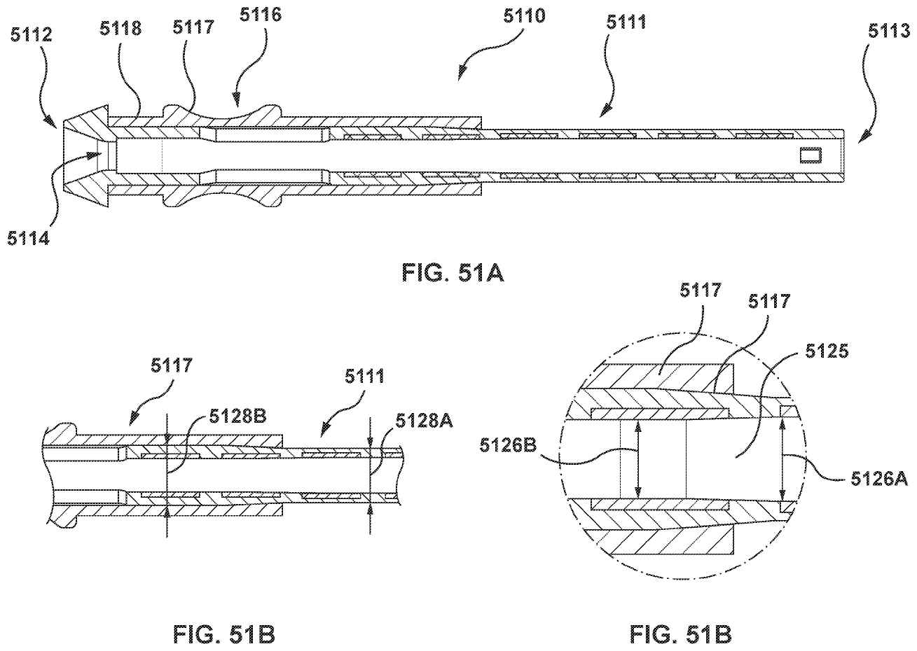

FIGS. 51A-51C show a capsule guide tube 5110 according to embodiments hereof. The capsule guide tube 5110 may be similar to the capsule guide tube 510 and the capsule guide tube 4910 described above. Therefore, all of the details of the capsule guide tube 5010 will not be described. The capsule guide tube 5110 generally includes a tube 5111 having a first (distal) end 5112, a second (proximal) end 5113, and a passageway or lumen 5114 extending from the first end 5112 to the second end 5113 such that the capsule guide tube 5110 can be disposed over the capsule 306 of the delivery system 300, as described above. The capsule guide tube 5110 further includes a grip (not shown) coupled to an exterior of the tube 5111, and a locking member 5016 configured to lock the capsule guide tube 5110 to the capsule 306 of the delivery system 300, as explained above. The locking member 5116 is disposed around the tube 5111 and includes a lock grip 5117 and a lock tube 5118. In the embodiment of FIGS. 51A-51C, the tube 5111 includes an internal taper 5125. The internal taper 5125 is located longitudinally approximately where a proximal portion of the lock tube 5118 is located with the locking member 5116 in the locked configuration shown in FIG. 51A. In particular, the lumen 5114 has a first diameter 5126A proximal of the internal taper 5125 and a second diameter 5126B distal of the internal taper 5125, wherein the second diameter 5126B is smaller than the first diameter 5126A. In a non-limiting embodiment, the first diameter 5126A may be approximately 6.1 mm and the second diameter 5126B may be approximately 5.9 mm. However, this is not meant to be limiting, and the actual sizes depend on the size of the capsule 306 of the delivery system 300. For example, and not by way of limitation, the diameters may be selected based on the amount of radial compression desired, such as 0.1 mm or 0.2 mm of radial compression. The tube 5111 further includes a ramp 5127 on an outer surface of the tube 511. In particular, the tube 5111 has a first outer diameter 5128A proximal of the ramp 5127 and a second outer diameter 5128B distal of the ramp 5127, wherein the second outer diameter 5128B is larger than the first outer diameter 51268. In a non-limiting embodiment, the first outer diameter 5128A may be approximately 8.9 mm and the second outer diameter 5128B may be approximately 10.3 mm. The axial location of the internal taper 5125 and the length of the external ramp 5127 allow for proximal compression of the capsule 306. The axial position of the internal taper 5125 provides an additional constraint to the area of the capsule 306 that expands least during loading, thus minimizing adverse impacts on loading forces. The internal taper 5125 provides for an interference fit with the capsule 306, rather than compressing the capsule 306. Although not described with respect to FIGS. 51A-51C, features of the other embodiments described herein may be combined with the capsule guide tube 5110, such as, but not limited to, the guide rails on the tube and grooves in the lock tube to prevent out of axis movement of the locking member 5116.

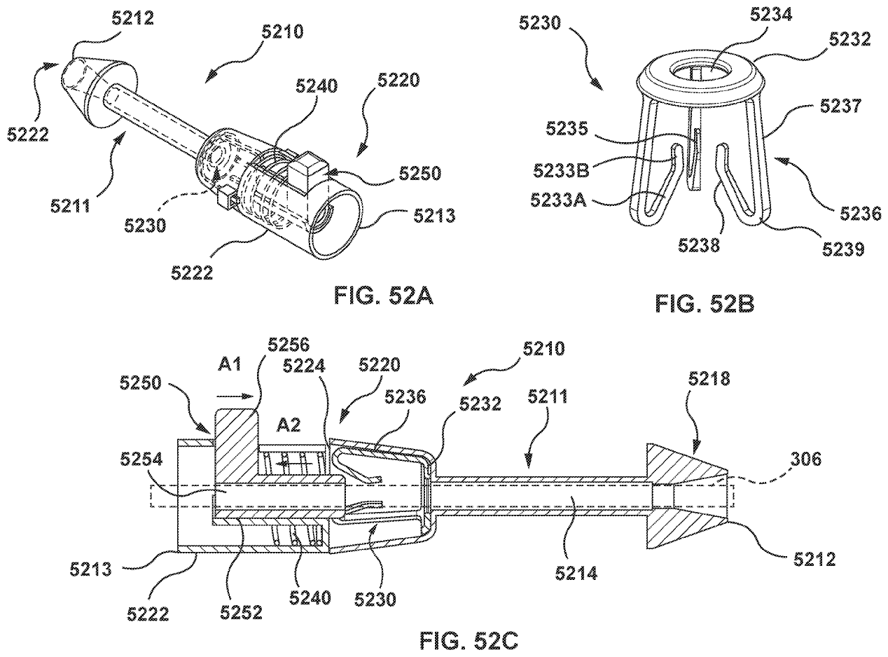

FIGS. 52A-52C show a capsule guide tube 5210 according to embodiments hereof. The capsule guide tube 5210 may be used in a similar fashion to the capsule guide tubes 110 and 510 described above. The capsule guide tube 5210 includes a tube 5211 and a locking member 5220. The capsule guide tube includes a first (distal) end 5212 and a second (proximal) end 5213. The first end 5212 of the capsule guide tube 5210 includes a flared portion 5218 flared in the proximal direction. The flared portion 5218 is configured to be received within a proximal end of the loading funnel 530. A lumen 5214 extends through the tube 5211, the locking member 5220, and the flared portion 5218. The lumen 5214 is configured to receive the capsule 306 (shown in phantom) therein.

The locking member 5220 is coupled to a proximal end of the tube 5211. The locking member 5216 includes a housing 5222 defining part of the lumen 5214. A spring lock 5230, a compression spring 5240, and a release slider 5250 are disposed within the housing 5222. The locking member 5220 is configured to lock the capsule guide tube 5210 onto the capsule 306 and release the capsule 306 from the capsule guide tube 5210 when desired.

The compression lock 5230 is shown in detail in FIG. 52B. The compression lock includes a base 5232 and compression legs 5236. The base 5232 includes a central opening 5234 configured to receive the capsule 306 of the delivery system 300 therethrough. The base 5232 abuts a distal end of the housing 5222, as shown in FIG. 52C. The compression legs 5236 apply a radially inward force to objects, such as the capsule 306 disposed therethrough. In the embodiment shown, the compression legs 5236 comprise three legs 5236. However, this is not meant to be limiting, and more or fewer legs 5236 may be utilized provided that the compression legs 5236 apply sufficient radially inward force to maintain the capsule guide tube 5210 locked to the capsule 306. In the embodiment shown, each compression leg 5236 is generally U-shaped. Therefore, each compression leg 5236 includes a first leg portion 5237 extending proximally from the base 5232, and bend 5239 at the proximal end of the first leg portion 5237, and a second leg portion 5238 extending distally and radially inwardly from the bend 5239. In the embodiment shown, each second leg portion 5239 includes a first portion 5233A extending radially inwardly and proximally from the bend 5239 and a section portion 5233B extending generally proximally from the first portion 5233A (i.e., generally parallel to a central longitudinal axis of the lumen 5214). The second portion 5233B provides a smooth surface for interaction with capsule 306. The second portions 5233B form a cylindrical opening 5235 with a diameter smaller than the diameter of the capsule 306 of the delivery system 300. Therefore, when the capsule is inserted through the cylindrical opening 5235, the second leg portions 5238 of the compression legs 5236 move radially apart, but exert a radially inward force on the capsule, thereby securing the capsule guide tube 5210 to the capsule 306.

The release slider 5250 is configured to splay the compression legs 5236 of the compression lock 5230 apart so as to release the capsule guide tube 5210 from the capsule 306. In the embodiment shown, the release slider 5250 includes a longitudinal portion 5254 having lumen 5254 extending therethrough. The lumen 5254 is configured to receive the capsule 306 of the delivery system 300 therein. However, the diameter of the lumen 5254 is larger than the diameter of the capsule 306 such that the longitudinal portion 5254 does not exert a radially inward force on the capsule 306 and the longitudinal portion 5254 can slide relative to the capsule 306 with the capsule 306 disposed in the lumen 5256. The release slide 5250 further includes an actuator 5256 configured to enable a user to actuate the release slider 5250. In the embodiment shown, the actuator 5256 extends substantially perpendicular to the longitudinal portion 5252 such that the actuator 5256 exits the housing 5222 such that a user may move the actuator 5256, and hence the release slider 5250 longitudinally, as indicated by the arrow A1 in FIG. 52C.

The compression spring 5240 of the locking member 5220 of the capsule guide tube 5210 is configured to maintain the release slider 5250, and in particular the longitudinal portion 5252 of the release slider 5250, spaced from the compression lock 5230, as shown in FIG. 52C. The compression spring 5240 is disposed in the housing 5222 between the actuator 5256 at a proximal end of the compression spring 5240 and a shoulder or wall 5222 of the housing 5222 at a distal end of the compression spring 5240. Thus, when the actuator 5256 is moved distally (i.e., in the direction of arrow A1), the compression spring 5240 compresses between the actuator 5256 and the shoulder 5222, and exerts a force proximally (i.e., in the direct of arrow A2).

In operation, in order to install the capsule guide tube 5210 over the capsule 306, the actuator 5256 is moved distally in the direction of the arrow A1. The movement must overcome the force of the compression spring 5240 acting in the direction of arrow A2. As the actuator 5256 is moved distally, the longitudinal portion 5252 is also moved distally. The longitudinal portion 5252 moves between the compression legs 5236 of the compression lock 5230, thereby splaying the compression legs 5236 apart from each other. The capsule guide tube 5210 may then be placed over the capsule 306 by relative axial movement of the capsule guide tube 5210 and the capsule 306. When the capsule guide tube 5210 is properly located, the actuator 5256 is released. The compression spring 5240 forces the actuator, and hence the release slide 5250 including the longitudinal portion 5252 proximally in the direction of the arrow A2. With the longitudinal portion 5252 being withdrawn from within the compression legs 5236, the compression legs 5236 move radially inwardly, thereby securing the capsule 306 therein. After the transcatheter heart valve prosthesis 200 is loaded into the capsule 306, the actuator 5256 may be moved distally such that the longitudinal portion 5252 of the release slider 5250 splays the compression legs 5236 apart, thereby releasing the capsule 306 from the capsule guide tube 5210. With the actuator 5256 actuated as described, the capsule guide tube 5210 can be removed from the capsule 306.

A capsule guide tube, such as the capsule guide tube 5210 with compression legs 5236 alleviates tight tolerances that may be required in other capsule guide tubes. Further, the capsule guide tube 5210 provides proximal compression (i.e., the compression legs 5236 are disposed at a proximal portion of the capsule 306). Proximal compression of the capsule guide tube 5210 onto the capsule 306 moves the compression forces on the capsule 306 proximally, i.e., away from the distal opening of the capsule 306. Loading forces when loading the transcatheter heart valve prosthesis 200 into the capsule 306 are highest near the distal end of the capsule 306. Having compression forces from the capsule guide tube on the capsule 306 at or near the highest loading forces (which is an expansion force on the capsule 306) increases loading forces. Therefore, moving the compression forces exerted on the capsule 306 by the capsule guide tube proximally reduces loading forces.

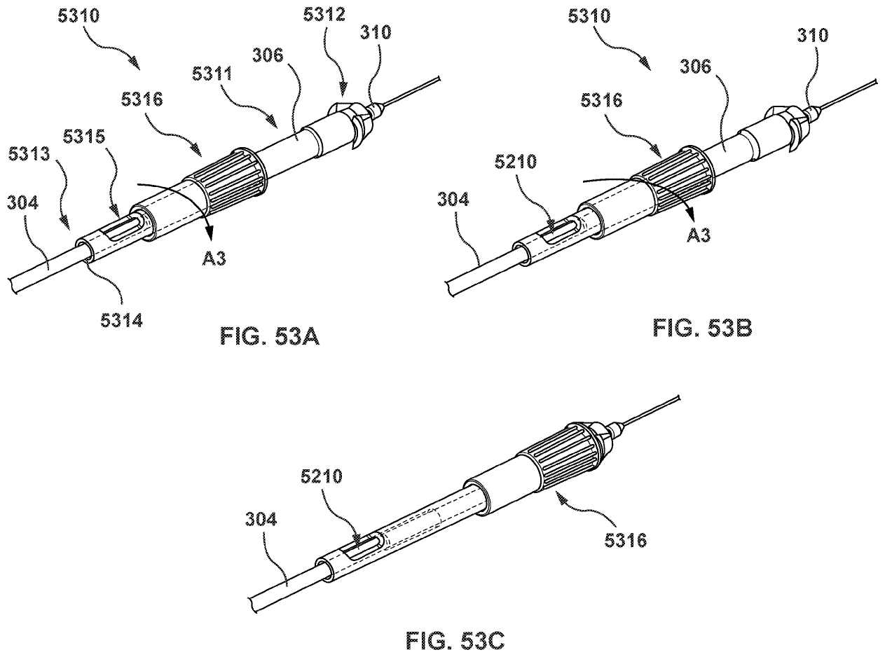

FIGS. 53A-53C show schematically a capsule guide tube 5310 according to embodiments hereof. The capsule guide tube 5310 may be similar to the capsule guide tube 510 described above. Therefore, all of the details of the capsule guide tube 5310 will not be described. The capsule guide tube 5310 generally includes a tube 5311 having a first (distal) end 5312, a second (proximal) end 5313, and a passageway or lumen 5314 extending from the first end 5312 to the second end 5313 such that the capsule guide tube 5310 can be disposed over the capsule 306 of the delivery system 300, as described above and shown in FIGS. 53A-53C. The capsule guide tube 5310 further includes a locking member 5316 configured to lock the capsule guide tube 5310 to the capsule 306 of the delivery system 300, as explained above. In the embodiment of FIGS. 53A-53C, the tube 5311 includes an opening 5315 adjacent the proximal end 5213 thereof. The opening 5315 extends through the wall of the tube 5311. The opening 5315 enables a user to grasp both the tube 5311 and the outer shaft 304 or the capsule 306 of the delivery system 300 through the opening 5315 when moving the locking member 5316 to prevent movement of the outer shaft 304 and capsule 306 during the locking procedure. The opening 5315 can be used with any of the embodiments described herein. As explained with respect to other embodiments herein, the locking member 5316 of the capsule guide tube 5310 is moved distally to lock the capsule guide tube 5310 onto the capsule 306. In the embodiment of FIGS. 53A-53C, the locking member 5316 is moved distally via rotation thereof, as indicated by the arrows A3 in FIGS. 53A-53B. Moving the locking member 5316 via rotation instead of linear translation may provide more stability and control of the movement. Further, rotation can typically better ensure that the locking action will be completed. In other words, there is a higher rick that a locking member moved via linear translation may not be fully slid into position.

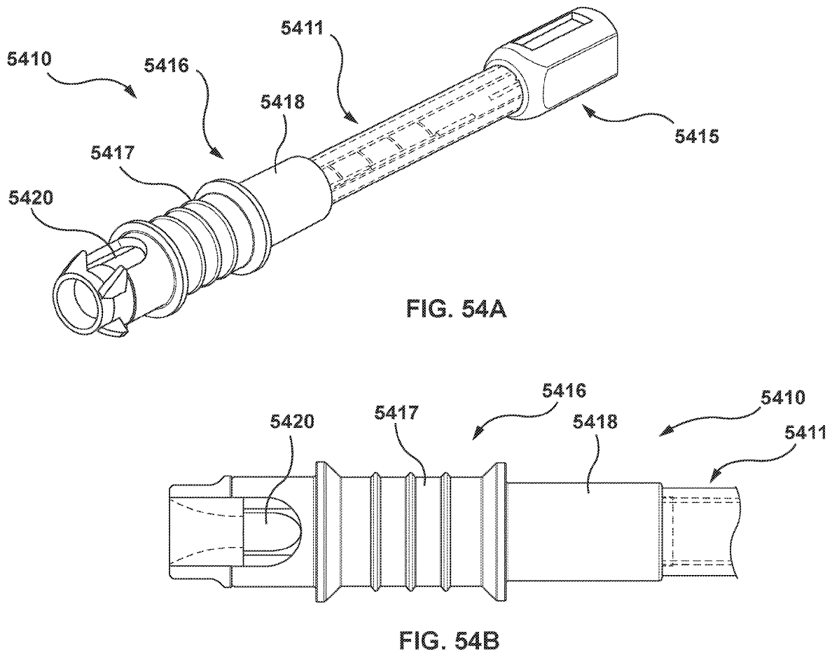

FIGS. 54A-54B show schematically a capsule guide tube 5410 according to embodiments hereof. The capsule guide tube 5410 may be similar to the capsule guide tube 510 described above. Therefore, all of the details of the capsule guide tube 5410 will not be described. The capsule guide tube 5410 generally includes a tube 5411, a grip 5415, and a locking member 5416 configured to lock the capsule guide tube 5410 to the capsule 306 of the delivery system 300, as explained above. Details of these features need not be repeated here. As explained with respect to other embodiments above, the locking member 5416 may include a lock grip 5417 and a lock tube 5418. In the embodiment of FIGS. 54A-54B, the locking member 5416 includes a window or opening 5320 through the wall of the lock tube 5418 at the distal end of the locking member 5416. The opening 5420 enables a user to visualize the distal end of the capsule 306 through the opening 5420 to ensure that the capsule guide tube 5410 and the capsule 306 are properly aligned when the locking member 5416 reaches the locking configuration shown in FIGS. 54A-54B. The opening 5420 may be incorporated into any of the embodiments described herein.

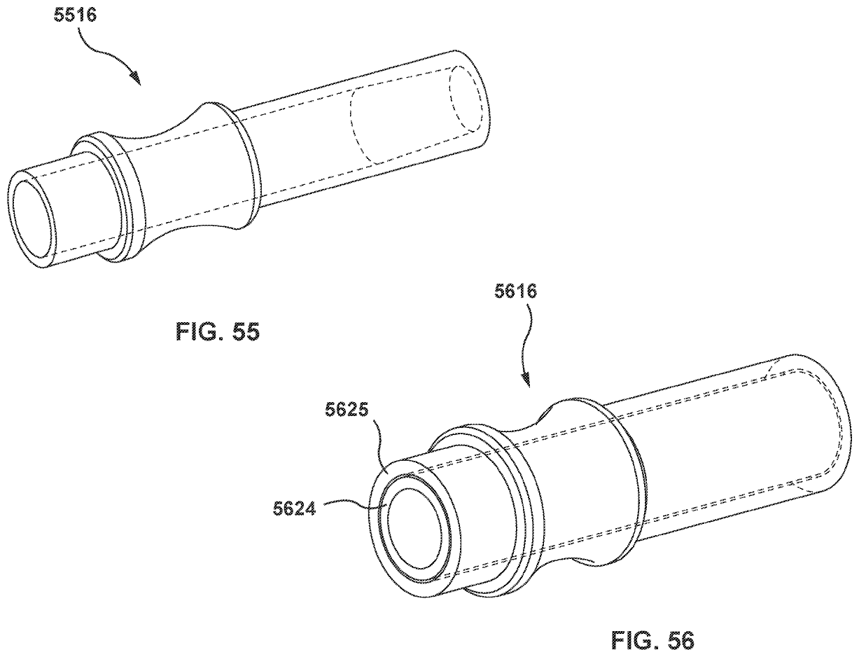

The locking members described herein may be formed of any material suitable for locking the capsule guide tube 510 to the capsule 306 of the delivery system 300. In an embodiment shown in FIG. 55 a locking member 5516 is formed from an elastic material, such as rubber. As explained above, the tube 511 of the capsule guide tube 510 may be formed of two halves. Thus, the elastic material of the locking member 5516 provides elastic compression of the tube 511 of capsule guide tube 510, thereby enabling transient deflection of the two halves of the tube 511 during loading of the transcatheter heart valve prosthesis 200 into the capsule 310. This transient deflection reduces loading forces when loading the transcatheter heart valve prosthesis 200 into the capsule 306. For example, and not by way of limitation, the material of the locking member 5516 may be rubbers (TPUs, NR, SBR, IIR, NBR, CR, EPDM, Q, FKM, AU, HNBR), foam-based polymers (PUR, PVC, PE, ABS), and/or polymers with Shore A hardness values between 50A and 60 (e.g., medical grade thermoplastic polyurethane, thermoplastic elastomers, polyurethan, and silicone).

In another embodiment hereof, shown in FIG. 56, a locking member 5616 may be formed of at least two materials. In particular, as shown in FIG. 56, an inner material layer 5624 and an outer material layer 5625. The inner material layer 5624 may be an elastic material, such as but not limited to rubbers (TPUs, NR, SBR, IIR, NBR, CR, EPDM, Q, FKM, AU, HNBR), foam-based polymers (PUR, PVC, PE, ABS), and/or polymers with Shore A hardness values between 50A and 60 (e.g., medical grade thermoplastic polyurethane, thermoplastic elastomers, polyurethan, and silicone). The outer material layer 5625 may be a hard polymer material such as, but not limited to high shore hardness polymers (ABS, PC,), metals (SS, Alum), glass, translucent polycarbonate (e.g. Makrolon 2458), acrylic (e.g., PMMA), and/or PETG. The elastic inner material layer 5624 enables transient deflection of the tube 511 of the capsule guide tube 510, as explained above with respect to FIG. 55. The hard outer material layer 5625 improves ergonomics and provides an outer limit of expansion of the tube 511, thereby preventing over-expansion of the tube 511 of the capsule guide tube 510.

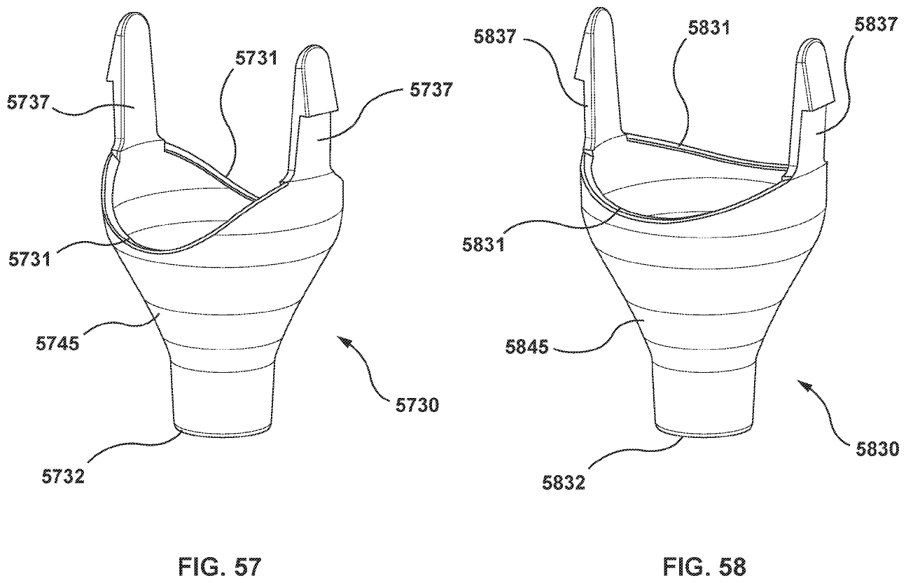

FIGS. 57 and 58 show two embodiments of loading cones 5730 and 5830. The loading cones 5730 and 5830 may be similar to the loading cones 530 described above with respect to FIGS. 17A and 17B. The loading cones 5730 and 5830 of FIGS. 57 and 58, respectively, include a main body 5745, 5845 with a sloped or contoured distal end 5731, 5831, respectively. In other words, as shown in FIGS. 57 and 58, the respective distal ends 5731, 5831 follow a contour from one of the locking arms 5737, 5837 towards the proximal end 5732, 5832 of the main body 5745, 5845, then back towards the other locking arm 5737, 5837. This contour is repeated on the opposite side of the locking arms. The contoured distal ends 5731, 5831 of the main body 5745, 5845 provide a gap between the loading cone 5730, 5830 and the loading ring 550 of the loading system 500. This gap enables air to escape the loading system 500, thereby mitigating any risk of an air embolism. Although each of FIGS. 57 and 58 show the contour repeated on opposite sides of the locking arms 5737, 5837, this is not meant to be limiting. In other embodiments, only one side of the distal end 5731, 5831 needs to be contoured. Further, as explained above with respect to FIGS. 1A and 2, instead of or in addition to the contoured distal end of the loading cone, the loading cone may include air vents (such as air vents 142 described above) for air release.

It should be understood that various embodiments disclosed herein may be combined in different combinations than the combinations specifically presented in the

1. A loading system for loading a heart valve prosthesis into a delivery system, the loading system comprising:
a loading cone, the loading cone including a passageway extending therethrough, the passageway including a tapered portion;
a loading ring configured to be coupled to the loading cone; and
a valve seat configured to be coupled to the loading ring, the valve seat being rotatable relative to the loading ring when the valve seat and the loading ring are coupled together, the valve seat configured to receive the heart valve prosthesis.

2. The loading system of claim 1, further comprising a capsule guide tube, wherein a distal end of the capsule guide tube is configured to be disposed adjacent a proximal end of the loading cone, and wherein the capsule guide tube is configured to be locked onto a capsule of the delivery system.

3. The loading system of claim 1, further comprising a tip guide tube configured to extend through the loading ring, the valve seat, and the loading cone.

4. The loading system of claim 1, wherein the tapered portion of the passageway is tapered in a proximal direction, such that a distal end of the tapered portion has a larger diameter than a proximal end of the tapered portion.

5. The loading system of claim 1, wherein the loading cone includes a plurality of ribs extending inwardly from an inner surface of the loading cone.

6. The loading system of claim 1, wherein the loading cone includes air vents extending through a surface thereof configured to release air from the loading cone.

7. The loading system of claim 1, wherein the loading cone includes a contoured distal edge such that a gap between the contoured distal edge and the loading ring enables air release from the loading cone.

8. The loading system of claim 1, wherein the loading ring includes a base and a central passageway extending through the base, and wherein the valve seat comprises a seat base having a central aperture extending therethrough, a wall extending from a periphery of the seat base in a first direction, and an aperture wall extending from a periphery of the central aperture in a second direction opposite the first direction, wherein the aperture wall is configured to be disposed in the central passageway of the loading ring such that the valve seat is rotatable relative to the loading ring.

9. The loading system of claim 8, wherein the seat base and the wall of the valve seat are configured to receive the heart valve prosthesis.

10. The loading system of claim 8, wherein the wall of the valve seat includes a plurality of ribs or a surface treatment configured to aid in the valve seat engaging the heart valve prosthesis such that rotation of the valve seat results in corresponding rotation of the heart valve prosthesis.

11. The loading system of claim 8, wherein the seat base includes a plurality of pockets configured to receive crowns at a first end of a frame of the heart valve prosthesis such that rotation of the valve seat results in corresponding rotation of the heart valve prosthesis.

12. The loading system of claim 8, wherein the wall of the valve seat comprises two wall sections separated by two wall slots, wherein the seat base of the valve seat further includes a living hinge configured to enable bending of the seat base to enable splaying of the two wall sections, and wherein squeezing the aperture wall radially inwardly splays the two wall sections radially outwardly to receive the heart valve prosthesis therein.

13. The loading system of claim 8, wherein the aperture wall of the valve seat includes a circumferential lip such that with the valve seat coupled to the loading ring, the circumferential lip inhibits longitudinal movement between the valve seat and the loading ring.

14. The loading system of claim 8, wherein the loading cone is non-rotatably coupled to the loading ring, and wherein the loading cone, the loading ring, and the valve seat are configured to receive the heart valve prosthesis therein, and with the loading cone, the loading ring, and the valve seat coupled together and the heart valve prosthesis loaded therein, the valve seat and the heart valve prosthesis are configured to rotate relative to the loading ring and the loading cone.

15. The loading system of claim 14, further comprising a tip guide tube configured to engage an inner surface of the aperture wall of the valve seat, wherein rotation of the tip guide tube rotates the valve seat relative to the loading ring.

16. The loading system of claim 15, wherein the tip guide tube includes a plurality of ridges configured to engage a corresponding plurality of slots in the aperture wall of the valve seat to rotatably engage the tip guide tube with the valve seat.

17. The loading system of claim 15, wherein the tip guide tube includes tactile feedback regarding the location of the tip guide tube relative to the valve seat coupled to the loading ring such that the tactile feedback enables a user to confirm that the tip guide tube is properly retracted distally a sufficient distance prior to advancing a capsule of the delivery system over the transcatheter heart valve prosthesis.

18. The loading system of claim 15, wherein the tip guide tube includes an internal spring or an external spring configured to push the tip guide tube distally relative to the delivery system.

19. The loading system of claim 2, wherein the capsule guide tube includes a tube comprising two longitudinal halves and a locking member configured to lock the tube onto the capsule of the delivery system, wherein the tube includes guide rails and the locking member includes grooves configured to receive the guide rails therein such that during longitudinal translation of the locking member relative to the tube, out of axis movement is prevented.

20. (canceled)

21. A method of loading a heart valve prosthesis into a delivery system, the method comprising:
sliding a capsule guide tube over a delivery system such that a distal end of the capsule guide tube adjacent an open distal end of a capsule of the delivery system;
coupling together a loading ring and a valve seat such that the valve seat is rotatable relative to the loading ring;
loading the heart valve prosthesis into the valve seat;
securing a loading cone to the loading ring with the valve seat secured to loading ring and the heart valve prosthesis disposed in the valve seat, thereby forming a loading system;
loading the loading system onto the delivery system;
rotating the valve seat and the heart valve prosthesis to align the heart valve prosthesis relative to the delivery system;
coupling the heart valve prosthesis to the delivery system;
translating the capsule distally to cover a portion of the heart valve prosthesis; and
further translating the capsule distally to such that the capsule and capsule guide tube push the loading cone, loading ring, and valve seat distally as the heart valve prosthesis is prevented from translating distally by being coupled to the delivery system such that the loading cone radially compresses the heart valve prosthesis, the heart valve prosthesis exits a proximal end of the loading cone and enters an open distal end of the capsule. description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.
